(19) United States
(12) Patent Application Publication
(10) Pub. No.: US 2024/0059682 A1
(43) Pub. Date: Feb. 22, 2024

(54) THYROID HORMONE RECEPTOR BETA AGONIST COMPOUNDS

(71) Applicant: Terns Pharmaceuticals, Inc., Foster City, CA (US)

(72) Inventors: Thorsten A. KIRSCHBERG, San Carlos, CA (US); Corey REEVES, Foster City, CA (US); Kevin KLUCHER, Foster City, CA (US); Martijn FENAUX, San Mateo, CA (US); Yingzi XU, Palo Alto, CA (US); F. Anthony ROMERO, Redwood City, CA (US); Randall HALCOMB, Foster City, CA (US)

(21) Appl. No.: 18/548,650

(22) PCT Filed: Mar. 2, 2022
(86) PCT No.: PCT/US2022/018575

§ 371 Date: Sep. 1, 2023

Related U.S. Application Data
(60) Provisional application No. 63/156,227, filed on Mar. 3, 2021.

(51) Int. Cl. C07D 413/12; C07D 403/12

(52) U.S. Cl. CPC C07D 413/12; C07D 403/12

(57) ABSTRACT

Provided herein are compounds, preferably thyroid hormone receptor beta (THR beta) agonist compounds, compositions thereof, and methods of their preparation, and methods of agonizing THR beta and methods for treating disorders ameliorated by activation of THR beta.

### CROSS-REFERENCE TO RELATED APPLICATIONS

This application claims the benefit of and the priority to U.S. Provisional Patent Application No. 63/156,227, filed Mar. 3, 2021, the disclosure of which is hereby incorporated by reference in its entirety for all purposes.

### FIELD

This invention relates to compounds, preferably thyroid hormone receptor beta (THR beta) agonist compounds, compositions thereof, and methods of their preparation, and methods of agonizing THR beta and methods for treating disorders ameliorated by activation of THR beta.

### BACKGROUND

The beneficial effects arising from treating hyperthyroid or hypothyroid patients with T3/T4 endogenous ligands or early analogs of these endogenous ligands have been described in the literature (Richardson Hill Jr., S. et al. J. Clin. Invest. 1960, 39, 523-533). These early studies, as well as similar follow-up studies, established the heart as a major organ for the manifestation of side effects of both hyperthyroidism and hypothyroidism (Klein, I. et al. Circulation, 2007, 1725-1735). In particular, tachycardia, hypertrophism, atrial dysrhythmias, and atrial fibrillation are serious concerns. In addition, increased bone turn-over leading to decreased bone mineral density has also been noted. Negative effects at both sites, heart and bone, have been linked to the agonism of the THR alpha isoform, whereas the beneficial effects of THR agonism in the liver are largely linked to the THR beta isoform (Sinha, R. A. et al. Nat. Rev. Endocrinology 2018, 14, 259-269). In addition, even targeted THR beta agonists can lead to suppression of the thyroid hormone axis (Erion, M. D., PNAS USA 2007, 104 (39), 15490-15495), which may lead to side effects ranging from depression and fatigue to muscle wasting and bone loss.

Diseases or disorders associated with THR beta include non-alcoholic steatohepatitis (NASH), non-alcoholic fatty liver disease (NAFLD), metabolic syndrome, dyslipidemia, hypertriglyceridemia, and hypercholesterolemia. There is a need for thyroid hormone analogs, such as those that are THR beta agonists, and preferably those that avoid the undesirable effects of hyperthyroidism and hypothyroidism, and maintain the beneficial effects of thyroid hormones, e.g., for the treatment for patients with non-alcoholic steatohepatitis (NASH). In particular, there is a need to develop new thyroid hormone analogs that are selective agonists for THR beta, and preferably those that avoid the undesirable effects associated with agonism of THR alpha and/or preferentially distributed in liver, and maintain the beneficial effects of thyroid hormones, e.g., for the treatment for patients with non-alcoholic steatohepatitis (NASH), non-alcoholic fatty liver disease (NAFLD), metabolic syndrome, dyslipidemia, hypertriglyceridemia, or hypercholesterolemia.

### SUMMARY

In some embodiments, provided herein is a compound of formula (I):

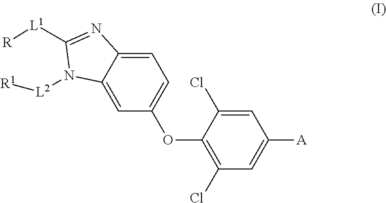

or a tautomer or stereoisomer thereof, or a pharmaceutically acceptable salt of any of the foregoing, wherein L¹, L², R, R¹, and A are as disclosed herein.

In some embodiments, provided herein is a pharmaceutical composition comprising a compound provided herein and a pharmaceutically acceptable excipient.

In some embodiments, provided herein is a method of agonizing thyroid hormone receptor beta (THR beta) comprising contacting either an effective amount of a compound provided herein, or an effective amount of a pharmaceutical composition provided herein, with the THR beta.

In some embodiments, provided herein is a method of treating a disorder which is ameliorated by activation of THR beta in a patient, comprising administering to the patient a therapeutically effective amount of a compound provided herein, or a therapeutically effective amount of a composition provided herein. In some embodiments, the disorder is non-alcoholic steatohepatitis (NASH), non-alcoholic fatty liver disease (NAFLD), metabolic syndrome, dyslipidemia, hypertriglyceridemia, or hypercholesterolemia. In some embodiments, the disorder is NASH.

### DETAILED DESCRIPTION

### Definitions

As used herein, the following definitions shall apply unless otherwise indicated. Further, if any term or symbol used herein is not defined as set forth below, it shall have its ordinary meaning in the art.

“Comprising” is intended to mean that the compositions and methods include the recited elements, but not excluding others. “Consisting essentially of”, when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of, e.g., other ingredients and substantial method steps recited. Embodiments defined by each of these transition terms are within the scope of this invention.

The term “about” refers to a variation of ±1%, ±3%, ±5%, or ±10% of the value specified. For example, “about 50” can in some embodiments includes a range of from 45 to 55. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. Reference to “about” a value or parameter herein includes (and describes) embodiments that are directed to that value or parameter per se. For example, description referring to “about X” includes description of “X”.

“Effective amount” or dose or “therapeutically effective amount” or dose of a compound or a composition refers to that amount of the compound or the composition that results in an intended result as desired based on the disclosure herein. Effective amounts can be determined by standard pharmaceutical procedures in cell cultures or experimental animals, e.g., and without limitation, by determining the LD₅₀ (the dose lethal to 50% of the population) and the ED₅₀ (the dose therapeutically effective in 50% of the population).

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Patient” refers to mammals and includes humans and non-human mammals. Examples of patients include, but are not limited to, mice, rats, hamsters, guinea pigs, pigs, rabbits, cats, dogs, goats, sheep, cows, and humans. In some embodiments, patient refers to a human.

“Pharmaceutically acceptable” refers to safe and non-toxic, preferably for in vivo, more preferably, for human administration.

“Pharmaceutically acceptable salt” refers to a salt that is pharmaceutically acceptable. A compound described herein may be administered as a pharmaceutically acceptable salt.

“Salt” refers to an ionic compound formed between an acid and a base. When the compound provided herein contains an acidic functionality, such salts include, without limitation, alkali metal, alkaline earth metal, and ammonium salts. As used herein, ammonium salts include salts containing protonated nitrogen bases and alkylated nitrogen bases. Exemplary and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionality, such salts include, without limitation, salts of organic acids, such as carboxylic acids and sulfonic acids, and mineral acids, such as hydrogen halides, sulfuric acid, phosphoric acid, and the like. Exemplary and non-limiting anions useful in pharmaceutically acceptable salts include oxalate, maleate, acetate, propionate, succinate, tartrate, chloride, sulfate, bisulfate, mono-, di-, and tribasic phosphate, mesylate, tosylate, and the like.

“Treatment” or “treating” is an approach for obtaining beneficial or desired results including clinical results. For purposes of this disclosure, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of this disclosure contemplate any one or more of these aspects of treatment.

An “isotopomer” of a compound is a compound in which one or more atoms of the compound have been replaced with isotopes of those same atoms. For example, where H has been replaced by D or T, or ¹²C has been replaced by ¹¹C, or ¹⁴N has been replaced by ¹⁵N. For example, and without limitation, replacement of with D can in some instances lead to reduced rates of metabolism and therefore longer half-lives. Replacement of H with T can provide radioligands potentially useful in binding studies. Replacement of ¹²C with the short-lived isotope ¹¹C can provide ligands useful in Positron Emission Tomography (PET) scanning. Replacement of ¹⁴N with ¹⁵N provides compounds that can be detected/monitored by ¹⁵N NMR spectroscopy. For example, an isotopomer of a compound containing —CH₂CH₃ is that compound but containing —CD₂CD₃ instead of the —CH₂CH₃.

Unless a specific isotope of an element is indicated in a formula, the disclosure includes all isotopologues of the compounds disclosed herein, such as, for example, deuterated derivatives of the compounds (where H can be ²H, i.e., D). Isotopologues can have isotopic replacements at any or at all locations in a structure, or can have atoms present in natural abundance at any or all locations in a structure.

“Stereoisomer” or “stereoisomers” refer to compounds that differ in the stereogenicity of the constituent atoms such as, without limitation, in the chirality of one or more stereocenters or related to the cis or trans configuration of a carbon-carbon or carbon-nitrogen double bond. Stereoisomers include enantiomers and diastereomers.

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Alkyl” refers to monovalent saturated aliphatic hydrocarbyl groups having from 1 to 12 carbon atoms, preferably from 1 to 10 carbon atoms, and more preferably from 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). Cₓ alkyl refers to an alkyl group having x number of carbon atoms.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl or C₆-C₂₀ aryl), 6 to 12 carbon ring atoms (i.e., C₆₋₁₂ aryl or C₆-C₁₂ aryl), or 6 to 10 carbon ring atoms (i.e., C₆₋₁₀ aryl or C₆-C₁₀ aryl). Examples of aryl groups include, without limitation, phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl or C₃-C₂₀ cycloalkyl), 3 to 12 ring carbon atoms (i.e., C₃₋₁₂ cycloalkyl or C₃-C₁₂ cycloalkyl), 3 to 10 ring carbon atoms (i.e., C₃₋₁₀ cycloalkyl or C₃-C₁₀ cycloalkyl), 3 to 8 ring carbon atoms (i.e., C₃₋₈ cycloalkyl or C₃-C₈ cycloalkyl), or 3 to 6 ring carbon atoms (i.e., C₃₋₆ cycloalkyl or or C₃-C₆ cycloalkyl). Monocyclic groups include, without limitation, cyclopropyl, cyclobutyl, cyclopentyl, cyclohexyl, cycloheptyl and cyclooctyl. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom.

“Heteroaryl” refers to an aromatic group having a single ring, multiple rings or multiple fused rings, with one or more ring heteroatoms independently selected from nitrogen, oxygen and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl) and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 5-12 membered ring systems, 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro and may comprise one or more (e.g., 1 to 3) oxo (═O) or N-oxide (N⁺—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ or C₂-C₂₀ heterocyclyl), 2 to 12 ring carbon atoms (i.e., C₂₋₁₂ or C₂-C₁₂ heterocyclyl), 2 to 10 ring carbon atoms (i.e., C₂₋₁₀ or C₂-C₁₀ heterocyclyl), 2 to 8 ring carbon atoms (i.e., C₂₋₈ or C₂-C₈ heterocyclyl), 3 to 12 ring carbon atoms (i.e., C₃₋₁₂ or C₃-C₁₂ heterocyclyl), 3 to 8 ring carbon atoms (i.e., C₃₋₈ or C₃-C₈ heterocyclyl), or 3 to 6 ring carbon atoms (i.e., C₃₋₆ or C₃-C₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. In certain instances, heterocyclyl includes 3-12 membered ring systems, 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. The term “heterocyclyl” also includes “spiroheterocyclyl” when there are two positions for substitution on the same carbon atom.

“Halo” or “halogen” refers to fluoro, chloro, bromo and iodo and preferably is fluoro or chloro.

“Hydroxy” or “hydroxyl” refers to the group —OH.

“Oxo” refers to the atom (═O) or (O).

The terms “optional” or “optionally” as used throughout the specification means that the subsequently described event or circumstance may but need not occur, and that the description includes instances where the event or circumstance occurs and instances in which it does not. For example, “the nitrogen atom is optionally oxidized to provide for the N-oxide (N→O) moiety” means that the nitrogen atom may but need not be oxidized, and the description includes situations where the nitrogen atom is not oxidized and situations where the nitrogen atom is oxidized.

“Optionally substituted” unless otherwise specified means that a group may be unsubstituted or substituted by one or more (e.g., 1, 2, 3, 4 or 5) of the substituents listed for that group in which the substituents may be the same of different, provided that the group's normal valence is not exceeded. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 2 to 5, 3 to 5, 2 to 3, 2 to 4, 3 to 4, 1 to 3, 1 to 4 or 1 to 5 substituents.

It is understood that the above definitions are not intended to include impermissible substitution patterns (e.g., methyl substituted with 4 fluoro groups). Such impermissible substitution patterns are well known to the skilled artisan.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present invention and are disclosed herein just as if each and every such sub-combination of chemical groups was individually and explicitly disclosed herein.

### Compounds

In some embodiments, provided herein is a compound of formula (I):

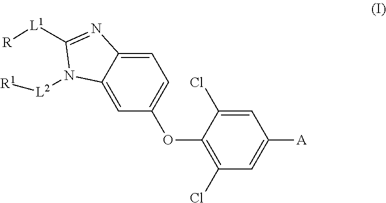

or a tautomer or stereoisomer thereof, or a pharmaceutically acceptable salt of each of the foregoing, wherein:
A is

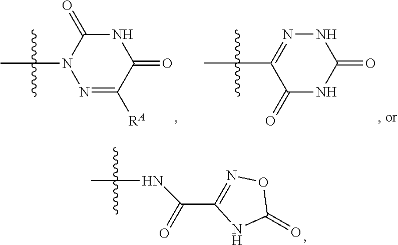

wherein R^{A} is H or —CN;
L¹ is a bond, —NR′—, —O—, —S—, or —S(O)₂—, wherein R′ is H or C₁-C₆ alkyl;
L² is a bond or —S(O)₂—;
R¹ is H, C₁-C₆ alkyl, C₆-C₁₀ aryl, 3-12 membered heterocyclyl, 5-12 membered heteroaryl, or C₃-C₆ cycloalkyl, wherein the C₁-C₆ alkyl, C₆-C₁₀ aryl, 3-12 membered heterocyclyl, 5-12 membered heteroaryl, and C₃-C₆ cycloalkyl are each independently optionally substituted by 1-5 R² groups;
R is H, C₁-C₆ alkyl, C₆-C₁₀ aryl, 3-12 membered heterocyclyl, 5-12 membered heteroaryl, or C₃-C₆ cycloalkyl, wherein the C₁-C₆ alkyl, C₆-C₁₀ aryl, 3-12 membered heterocyclyl, 5-12 membered heteroaryl, and C₃-C₆ cycloalkyl are each independently optionally substituted by 1-5 R² groups; and
each R² is independently halogen, C₁-C₆ alkyl, C₃-C₆ cycloalkyl, C₁-C₆ haloalkyl, C₁-C₆ alkyl-OH, —NH₂, —CN, or hydroxyl, provided that
when L¹ is a bond and R is H, then A is

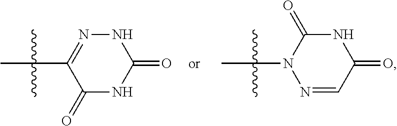 [SMILES: *c1n[nH]c(=O)[nH]c1=O ; *n1ncc(=O)[nH]c1=O]

and
when L¹ is —O—, R is H, and A is

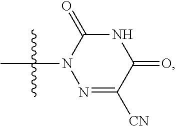 [SMILES: *n1nc(C#N)c(=O)[nH]c1=O]

then R¹ is C₂-C₆ alkyl, C₆-C₁₀ aryl, 3-12 membered heterocyclyl, 5-12 membered heteroaryl, or C₃-C₆ cycloalkyl, wherein the C₂-C₆ alkyl, C₆-C₁₀ aryl, 3-12 membered heterocyclyl, 5-12 membered heteroaryl, and C₃-C₆ cycloalkyl are each independently optionally substituted by 1-5 R² groups.

In some embodiments, provided is a compound of formula (I), or a tautomer or an N-oxide thereof, or an isotopomer of each thereof, or a stereoisomer of the aforesaid, or a pharmaceutically acceptable salt of each of the foregoing, or a solvate of each of the preceding. In some embodiments, provided is a compound of formula (I) or a pharmaceutically acceptable salt thereof.

In some embodiments, the compound of formula (I) is not a compound selected from the compounds in Table 1X, or a tautomer or stereoisomer thereof, or a pharmaceutically acceptable salt of each of the foregoing.

TABLE 1X

| No. | Name |
|---|---|
| 1x | N-(3,5-dichloro-4-((3-cyclopropyl-2-oxo-2,3-dihydro-1H-benzo[d]imidazol-5- |
| | yl)oxy)phenyl)-5-oxo-4,5-dihydro-1,2,4-oxadiazole-3-carboxamide |
| 2x | 2-(3,5-dichloro-4-((1-cyclopropyl-2-methoxy-1H-benzo[d]imidazol-6- |
| | yl)oxy)phenyl)-3,5-dioxo-2,3,4,5-tetrahydro-1,2,4-triazine-6-carbonitrile |
| 3x | N-(3,5-dichloro-4-((2-methoxy-1-methyl-1H-benzo[d]imidazol-6-yl)oxy)phenyl)- |
| | 5-oxo-4,5-dihydro-1,2,4-oxadiazole-3-carboxamide |
| 4x | N-(3,5-dichloro-4-((3-isopropyl-2-oxo-2,3-dihydro-1H-benzo[d]imidazol-5- |
| | yl)oxy)phenyl)-5-oxo-4,5-dihydro-1,2,4-oxadiazole-3-carboxamide |
| 5x | 2-(3,5-dichloro-4-((3-isopropyl-2-oxo-2,3-dihydro-1H-benzo[d]imidazol-5- |
| | yl)oxy)phenyl)-3,5-dioxo-2,3,4,5-tetrahydro-1,2,4-triazine-6-carbonitrile |
| 6x | N-(3,5-dichloro-4-((1-isopropyl-2-methoxy-1H-benzo[d]imidazol-6- |
| | yl)oxy)phenyl)-5-oxo-4,5-dihydro-1,2,4-oxadiazole-3-carboxamide |
| 7x | N-(3,5-dichloro-4-((1-cyclopropyl-2-methoxy-1H-benzo[d]imidazol-6- |
| | yl)oxy)phenyl)-5-oxo-4,5-dihydro-1,2,4-oxadiazole-3-carboxamide |
| 8x | 2-(3,5-dichloro-4-((3-cyclopropyl-2-oxo-2,3-dihydro-1H-benzo[d]imidazol-5- |
| | yl)oxy)phenyl)-3,5-dioxo-2,3,4,5-tetrahydro-1,2,4-triazine-6-carbonitrile |
| 9x | N-(4-((1-(tert-butyl)-2-methoxy-1H-benzo[d]imidazol-6-yl)oxy)-3,5- |
| | dichlorophenyl)-5-oxo-4,5-dihydro-1,2,4-oxadiazole-3-carboxamide |
| 10x | N-(4-((3-(tert-butyl)-2-oxo-2,3-dihydro-1H-benzo[d]imidazol-5-yl)oxy)-3,5- |
| | dichlorophenyl)-5-oxo-4,5-dihydro-1,2,4-oxadiazole-3-carboxamide |
| 11x | N-(3,5-dichloro-4-((2-methoxy-1-(1-methylcyclopropyl)-1H-benzo[d]imidazol-6- |
| | yl)oxy)phenyl)-5-oxo-4,5-dihydro-1,2,4-oxadiazole-3-carboxamide |
| 12x | 2-(3,5-dichloro-4-((2-methoxy-1-(1-methylcyclopropyl)-1H-benzo[d]imidazol-6- |
| | yl)oxy)phenyl)-3,5-dioxo-2,3,4,5-tetrahydro-1,2,4-triazine-6-carbonitrile |
| 13x | N-(3,5-dichloro-4-((3-(1-methylcyclopropyl)-2-oxo-2,3-dihydro-1H- |
| | benzo[d]imidazol-5-yl)oxy)phenyl)-5-oxo-4,5-dihydro-1,2,4-oxadiazole-3- |
| | carboxamide |
| 14x | 2-(3,5-dichloro-4-((3-(1-methylcyclopropyl)-2-oxo-2,3-dihydro-1H- |
| | benzo[d]imidazol-5-yl)oxy)phenyl)-3,5-dioxo-2,3,4,5-tetrahydro-1,2,4-triazine-6- |
| | carbonitrile |
| 15x | 2-(4-((1-(tert-butyl)-2-methoxy-1H-benzo[d]imidazol-6-yl)oxy)-3,5- |
| | dichlorophenyl)-3,5-dioxo-2,3,4,5-tetrahydro-1,2,4-triazine-6-carbonitrile |
| 16x | 2-(4-((3-(tert-butyl)-2-oxo-2,3-dihydro-1H-benzo[d]imidazol-5-yl)oxy)-3,5- |
| | dichlorophenyl)-3,5-dioxo-2,3,4,5-tetrahydro-1,2,4-triazine-6-carbonitrile |
| 17x | N-(3,5-dichloro-4-((3-(1-(difluoromethyl)cyclopropyl)-2-oxo-2,3-dihydro-1H- |
| | benzo[d]imidazol-5-yl)oxy)phenyl)-5-oxo-4,5-dihydro-1,2,4-oxadiazole-3- |
| | carboxamide |
| 18x | 2-(3,5-dichloro-4-((3-(1-(difluoromethyl)cyclopropyl)-2-oxo-2,3-dihydro-1H- |
| | benzo[d]imidazol-5-yl)oxy)phenyl)-3,5-dioxo-2,3,4,5-tetrahydro-1,2,4-triazine-6- |
| | carbonitrile |
| 19x | N-(3,5-dichloro-4-((3-methyl-2-oxo-2,3-dihydro-1H-benzo[d]imidazol-5- |
| | yl)oxy)phenyl)-5-oxo-4,5-dihydro-1,2,4-oxadiazole-3-carboxamide |

In some embodiments of a compound of formula (I) or any variation thereof, A is

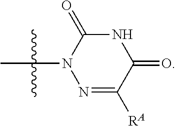

In some embodiments, R^{A} is H. In some embodiments, R^{A} is —CN. In some embodiments, A is

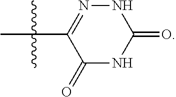 [SMILES: *c1n[nH]c(=O)[nH]c1=O]

In some embodiments, A is

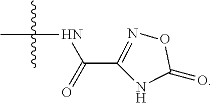 [SMILES: *NC(=O)c1noc(=O)[nH]1]

In some embodiments, the compound of formula (I) is a compound of formula (II):

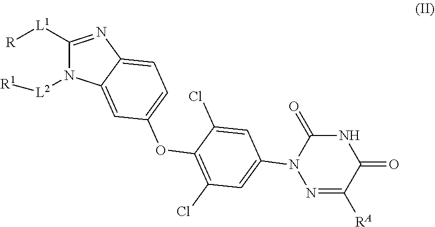

wherein L¹, L², R, and R¹ are as defined herein for formula (I). In some embodiments, R^{A} is H. In some embodiments, R^{A} is —CN.

In some embodiments, the compound of formula (I) is a compound of formula (III):

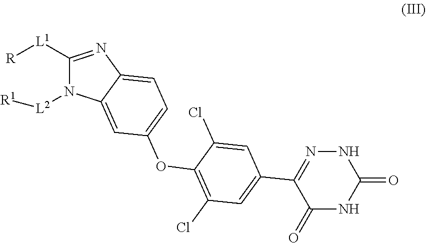

wherein L¹, L², R, and R¹ are as defined herein for formula (I).

In some embodiments, the compound of formula (I) is a compound of formula (IV):

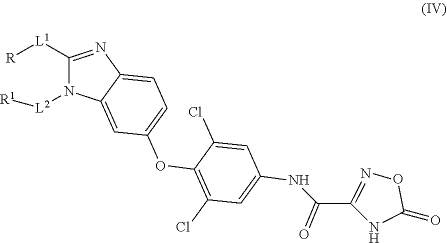

wherein L¹, L², R, and R¹ are as defined herein for formula (I).

In some embodiments of a compound of formula (I) or any variation thereof, L¹ is a bond. In some embodiments, L¹ is —NR′—. In some embodiments, L¹ is —NR′—, wherein R′ is H. In some embodiments, L¹ is —NR′—, wherein R′ is C₁-C₆ alkyl such as methyl. In some embodiments, L¹ is —S—. In some embodiments, L¹ is —S(O)₂—. In some embodiments of a compound of formula (I) or any variation thereof, L² is a bond. In some embodiments, L² is —S(O)₂—. In some embodiments of a compound of formula (I) or any variation thereof, L¹ is a bond; and L² is a bond. In some embodiments, L¹ is a bond; and L² is a —S(O)₂—. In some embodiments, L¹ is —NR′—; and L² is a bond. In some embodiments, L¹ is —NR′—; and L² is a —S(O)₂—. In some embodiments, L¹ is —O—; and L² is a bond. In some embodiments, L¹ is —O—; and L² is a —S(O)₂—. In some embodiments, L¹ is —S—; and L² is a bond. In some embodiments, L¹ is —S—; and L² is a —S(O)₂—. In some embodiments, L¹ is —S(O)₂—; and L² is a bond. In some embodiments, L¹ is —S(O)₂—; and L² is a —S(O)₂—.

In some embodiments, a compound of formula (I) is of formula (I-1), (I-2), (I-3), (I-4), (I-5), (I-6), (I-7), (I-8), (I-9), or (I-10).

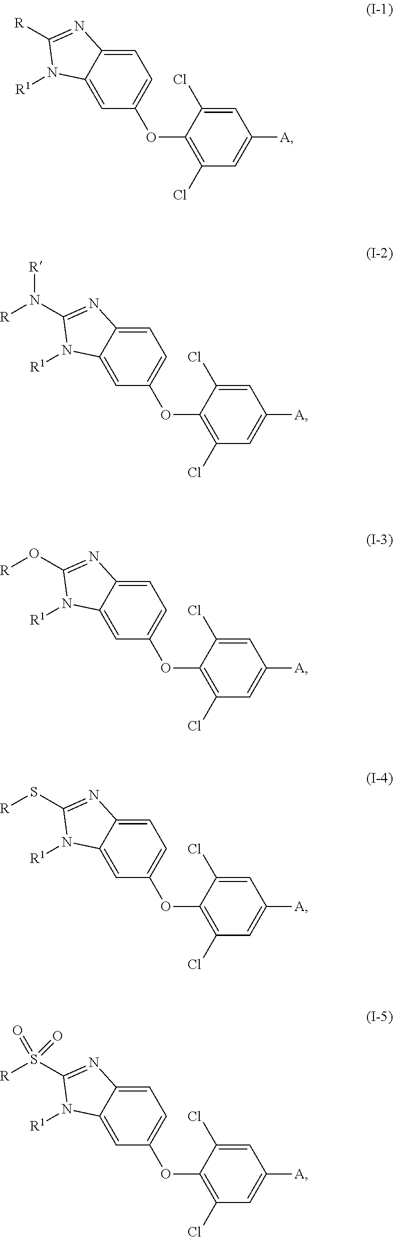

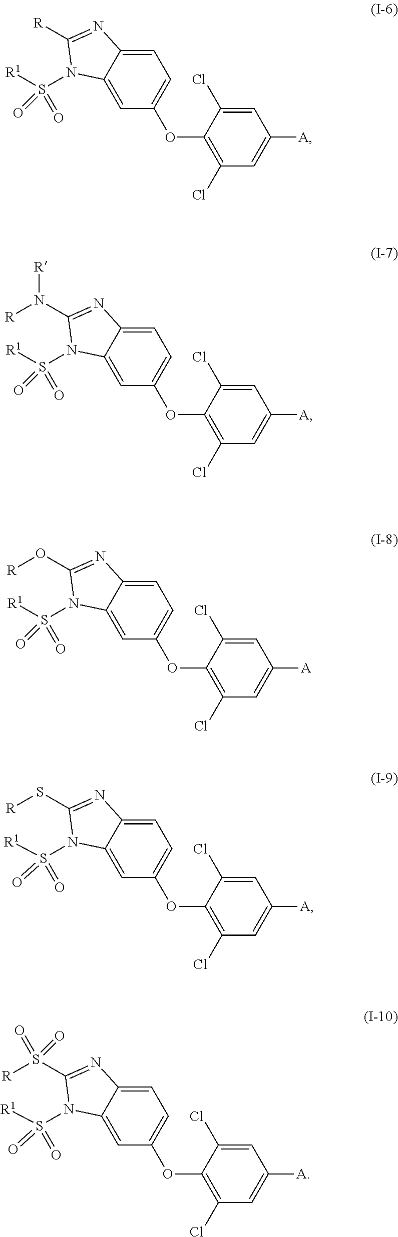

In some embodiments of a compound of formula (I) or any variation thereof, R¹ is H, C₁-C₃ alkyl, or C₃-C₆ cycloalkyl, wherein the C₁-C₃ alkyl and C₃-C₆ cycloalkyl are each independently optionally substituted by 1-3 R² groups. In some embodiments, R¹ is H, methyl, or ethyl. In some embodiments, R¹ is H, cyclopropyl, methyl, isopropyl, t-butyl, or ethyl. In some embodiments, R¹ is H. In some embodiments, R¹ is C₁-C₆ alkyl which is optionally substituted by 1-5 R² groups. In some embodiments, R¹ is C₁-C₆ alkyl which is unsubstituted. In some embodiments, R¹ is C₁-C₃ alkyl which is optionally substituted by 1-3 R² groups. In some embodiments, R¹ is C₁-C₃ alkyl which is unsubstituted. In some embodiments, R¹ is methyl or ethyl. In some embodiments, R¹ is methyl, ethyl, isopropyl, or t-butyl. In some embodiments, R¹ is methyl. In some embodiments, R¹ is ethyl. In some embodiments, R¹ is isopropyl. In some embodiments, R¹ is t-butyl. In some embodiments, R¹ is C₃-C₆ cycloalkyl which is optionally substituted by 1-5 R² groups. In some embodiments, R¹ is C₃-C₆ cycloalkyl which is unsubstituted. In some embodiments, R¹ is C₃-C₅ cycloalkyl which is optionally substituted by 1-3 R² groups. In some embodiments, R¹ is C₃-C₅ cycloalkyl which is unsubstituted. In some embodiments, R¹ is cyclopropyl. In some embodiments, R¹ is cyclopropyl which is substituted by 1 R² group. In some embodiments, R¹ is cyclopropyl or cyclobutyl, each of which is independently optionally substituted by 1 R² group. In some embodiments, R¹ is cyclopropyl which is substituted by 1 R² group, wherein the R² group is C₁-C₆ alkyl or C₁-C₆ haloalkyl. In some embodiments, R¹ is

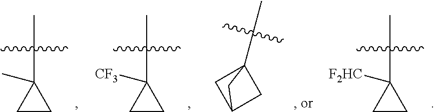

In some embodiments, is

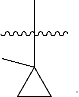 [SMILES: *C1(C)CC1]

In some embodiments, R¹ is

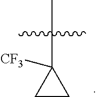 [SMILES: *C1(C(F)(F)F)CC1]

In some embodiments, R¹ is

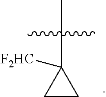 [SMILES: *C1(C(F)F)CC1]

In some embodiments, R¹ is

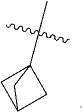 [SMILES: *C12CC(C1)C2]

In some embodiments, R¹ is 3-12 membered heterocyclyl optionally substituted by 1-5 R² groups. In some embodiments, R¹ is 3-12 membered heterocyclyl. In some embodiments, R¹ is 5-6 membered heterocyclyl optionally substituted by 1-5 R² groups. In some embodiments, R¹ is 5-6 membered heterocyclyl. In some embodiments, R¹ is C₆-C₁₀ aryl optionally substituted by 1-5 R² groups. In some embodiments, R¹ is C₆-C₁₀ aryl. In some embodiments, R¹ is phenyl optionally substituted by 1-5 R² groups. In some embodiments, R¹ is phenyl. In some embodiments, R¹ is 5-12 membered heteroaryl optionally substituted by 1-5 R² groups. In some embodiments, R¹ is 5-12 membered heteroaryl. In some embodiments, R¹ is 5-6 membered heteroaryl optionally substituted by 1-5 R² groups. In some embodiments, R¹ is 5-6 membered heteroaryl.

In some embodiments of a compound of formula (I) or any variation thereof, R is C₁-C₆ alkyl optionally substituted by 1-3 R² groups. In some embodiments, R is C₁-C₃ alkyl optionally substituted by 1-3 R² groups. In some embodiments, R is C₁-C₃ alkyl optionally substituted by 1-3 R² groups. In some embodiments, R is C₂-C₆ alkyl optionally substituted by 1-3 R² groups. In some embodiments, R is methyl, ethyl, n-propyl, isopropyl, n-butyl, or isobutyl, each of which is optionally substituted by 1-3 R² groups. In some embodiments, R is methyl, ethyl, n-propyl, isopropyl, n-butyl, or isobutyl. In some embodiments, R is ethyl, n-propyl, isopropyl, n-butyl, or isobutyl, each of which is optionally substituted by 1-3 R² groups. In some embodiments, R is methyl or ethyl, each of which is optionally substituted by 1-3 R² groups. In some embodiments, R is methyl optionally substituted by 1-3 R² groups. In some embodiments, R is methyl. In some embodiments, R is ethyl optionally substituted by 1-3 R². In some embodiments, R is ethyl. In some embodiments, R is n-propyl optionally substituted by 1-3 R². In some embodiments, R is n-propyl. In some embodiments, R is isopropyl optionally substituted by 1-3 R². In some embodiments, R is isopropyl. In some embodiments, R is n-butyl optionally substituted by 1-3 R². In some embodiments, R is n-butyl. In some embodiments, R is isobutyl optionally substituted by 1-3 R². In some embodiments, R is isobutyl. In some embodiments, R is 3-12 membered heterocyclyl optionally substituted by 1-5 R² groups. In some embodiments, R is 3-12 membered heterocyclyl. In some embodiments, R is 5-6 membered heterocyclyl optionally substituted by 1-5 R² groups. In some embodiments, R is 5-6 membered heterocyclyl. In some embodiments, R is C₆-C₁₀ aryl optionally substituted by 1-5 R² groups. In some embodiments, R is C₆-C₁₀ aryl. In some embodiments, R is phenyl optionally substituted by 1-5 R² groups. In some embodiments, R is phenyl. In some embodiments, R is 5-12 membered heteroaryl optionally substituted by 1-5 R² groups. In some embodiments, R is 5-12 membered heteroaryl. In some embodiments, R is 5-6 membered heteroaryl optionally substituted by 1-5 R² groups. In some embodiments, R is 5-6 membered heteroaryl.

In some embodiments of a compound of formula (I) or any variation thereof, each R², where present, is independently halogen, C₁-C₃ alkyl, C₁-C₃ haloalkyl, C₁-C₃ alkyl-OH, —NH₂, —CN, or hydroxyl. In some embodiments, each R², where present, is independently Cl, F, —CH₃, —CF₃, —CHF₂, —CH₂OH, —NH₂, —CN, or hydroxyl. In some embodiments, where R² is present, at least one R² is halogen, such as Cl or F. In some embodiments, where R² is present, at least one R² is Cl. In some embodiments, where R² is present, at least one R² is F. In some embodiments, where R² is present, at least one R² is C₁-C₃ alkyl, such as —CH₃, —CH₂CH₃, —CH₂CH₂CH₃, or —CH(CH₃)₂. In some embodiments, where R² is present, at least one R² is —CH₃. In some embodiments, where R² is present, at least one R² is C₃-C₆ cycloalkyl. In some embodiments, where R² is present, at least one R² is C₁-C₃ haloalkyl. In some embodiments, where R² is present, at least one R² is C₁-C₃ haloalkyl having 1-3 halogen atoms. In some embodiments, where R² is present, at least one R² is C₁-C₃ haloalkyl having 1 halogen atom. In some embodiments, where R² is present, at least one R² is C₁-C₃ haloalkyl having 2 halogen atoms. In some embodiments, where R² is present, at least one R² is C₁-C₃ haloalkyl having 3 halogen atoms. In some embodiments, where R² is present, at least one R² is —CF₃. In some embodiments, where R² is present, at least one R² is —CHF₂. In some embodiments, where R² is present, at least one R² is C₁-C₃ alkyl-OH. In some embodiments, where R² is present, at least one R² is —CH₂OH. In some embodiments, where R² is present, at least one R² is —NH₂. In some embodiments, where R² is present, at least one R² is —CN. In some embodiments, where R² is present, at least one R² is hydroxyl.

In some embodiments, the compound of formula (I) is an agonist of THR beta. In some embodiments, the compound of formula (I) is an agonist of THR beta and is selective over THR alpha. In some embodiments, the compound of formula (I) has at least 2-fold selectivity for THR beta over THR alpha. In some embodiments, the compound of formula (I) has at least 5-fold selectivity for THR beta over THR alpha. In some embodiments, the compound of formula (I) has at least 10-fold selectivity for THR beta over THR alpha. In some embodiments, the compound of formula (I) has at least 20-fold selectivity for THR beta over THR alpha. In some embodiments, the compound of formula (I) has at least 50-fold selectivity for THR beta over THR alpha. In some embodiments, the compound of formula (I) has at least 75-fold selectivity for THR beta over THR alpha. In some embodiments, the compound of formula (I) has at least 100-fold selectivity for THR beta over THR alpha. In some embodiments, the compound of formula (I) has at least 2-, 3-, 4-, 5-, 6-, 7-, 8-, 9-, 10-, 15-, 20-, 25-, 30-, 35-, 40-, 45-, 50-, 55-, 60-, 65-, 70-, 75-, 80-, 85-, 90-, 95-, or 100-fold selectivity for THR beta over THR alpha. In any such embodiment, in some embodiments selectivity is assessed via a biochemical assay, such as the TR-FRET assay described in Example B 1. In some embodiments, in another aspect selectivity is assessed via a biochemical assay, such as the RXR heterodimer assay described in Example B2.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to A of formula (I) may be combined with every description, variation, embodiment or aspect of R, L¹, L², R¹, and R² the same as if each and every combination were specifically and individually listed. It is also understood that all descriptions, variations, embodiments or aspects of formula (I), where applicable, apply equally to other formulae detailed herein, and are equally described, the same as if each and every description, variation, embodiment or aspect were separately and individually listed for all formulae. For example, it is understood that, all descriptions, variations, embodiments or aspects of formula (I), where applicable, apply equally to any of formulae as detailed herein, such as formulae (I-1)-(I-10), (II), (III), and (IV) and are equally described, the same as if each and every description, variation, embodiment or aspect were separately and individually listed for all formulae.

In some embodiments, provided is a compound selected from the compounds in Table 1, or pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

In one embodiment, provided herein is a compound selected from those tabulated below in Table 1:

TABLE 1

| Example | Structure |
|---|---|
| 1 | 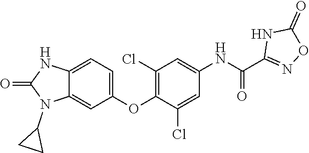 [SMILES: O=C(Nc1cc(Cl)c(Oc2ccc3[nH]c(=O)n(C4CC4)c3c2)c(Cl)c1)c1noc(=O)[nH]1] |
| 2 | 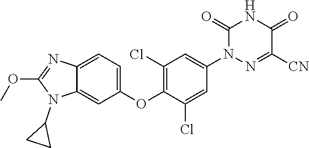 [SMILES: COc1nc2ccc(Oc3c(Cl)cc(-n4nc(C#N)c(=O)[nH]c4=O)cc3Cl)cc2n1C1CC1] |
| 3 | 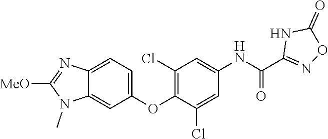 [SMILES: COc1nc2ccc(Oc3c(Cl)cc(NC(=O)c4noc(=O)[nH]4)cc3Cl)cc2n1C] |
| 4 | 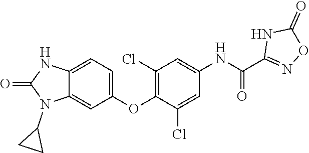 [SMILES: O=C(Nc1cc(Cl)c(Oc2ccc3[nH]c(=O)n(C4CC4)c3c2)c(Cl)c1)c1noc(=O)[nH]1] |
| 5 | 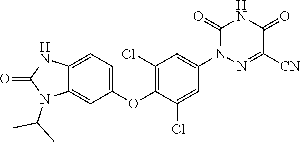 [SMILES: CC(C)n1c(=O)[nH]c2ccc(Oc3c(Cl)cc(-n4nc(C#N)c(=O)[nH]c4=O)cc3Cl)cc21] |
| 6 | 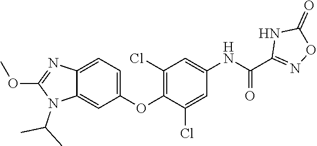 [SMILES: COc1nc2ccc(Oc3c(Cl)cc(NC(=O)c4noc(=O)[nH]4)cc3Cl)cc2n1C(C)C] |
| 7 | 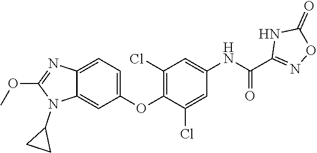 [SMILES: COc1nc2ccc(Oc3c(Cl)cc(NC(=O)c4noc(=O)[nH]4)cc3Cl)cc2n1C1CC1] |
| 8 | 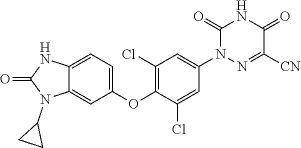 [SMILES: N#Cc1nn(-c2cc(Cl)c(Oc3ccc4[nH]c(=O)n(C5CC5)c4c3)c(Cl)c2)c(=O)[nH]c1=O] |
| 9 | 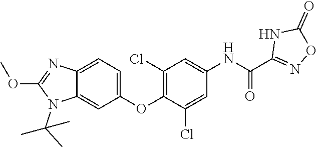 [SMILES: COc1nc2ccc(Oc3c(Cl)cc(NC(=O)c4noc(=O)[nH]4)cc3Cl)cc2n1C(C)(C)C] |
| 10 | 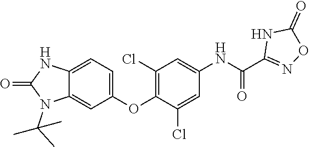 [SMILES: CC(C)(C)n1c(=O)[nH]c2ccc(Oc3c(Cl)cc(NC(=O)c4noc(=O)[nH]4)cc3Cl)cc21] |
| 11 | 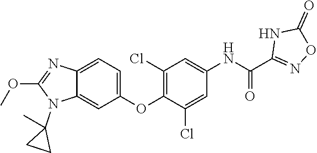 [SMILES: COc1nc2ccc(Oc3c(Cl)cc(NC(=O)c4noc(=O)[nH]4)cc3Cl)cc2n1C1(C)CC1] |
| 12 | 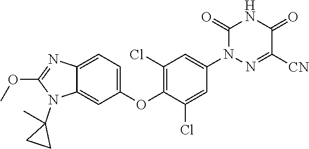 [SMILES: COc1nc2ccc(Oc3c(Cl)cc(-n4nc(C#N)c(=O)[nH]c4=O)cc3Cl)cc2n1C1(C)CC1] |
| 13 | 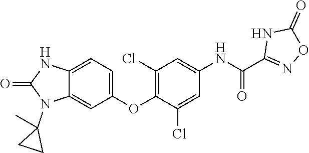 [SMILES: CC1(n2c(=O)[nH]c3ccc(Oc4c(Cl)cc(NC(=O)c5noc(=O)[nH]5)cc4Cl)cc32)CC1] |
| 14 | 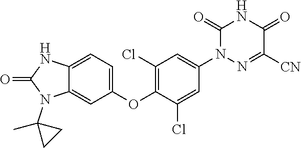 [SMILES: CC1(n2c(=O)[nH]c3ccc(Oc4c(Cl)cc(-n5nc(C#N)c(=O)[nH]c5=O)cc4Cl)cc32)CC1] |
| 15 | 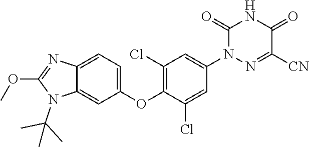 [SMILES: COc1nc2ccc(Oc3c(Cl)cc(-n4nc(C#N)c(=O)[nH]c4=O)cc3Cl)cc2n1C(C)(C)C] |
| 16 | 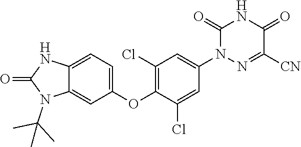 [SMILES: CC(C)(C)n1c(=O)[nH]c2ccc(Oc3c(Cl)cc(-n4nc(C#N)c(=O)[nH]c4=O)cc3Cl)cc21] |
| 17 | 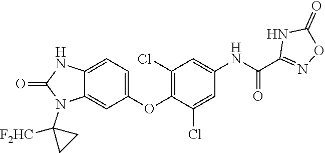 [SMILES: O=C(Nc1cc(Cl)c(Oc2ccc3[nH]c(=O)n(C4(C(F)F)CC4)c3c2)c(Cl)c1)c1noc(=O)[nH]1] |
| 18 | 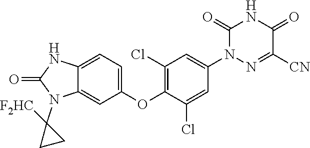 [SMILES: N#Cc1nn(-c2cc(Cl)c(Oc3ccc4[nH]c(=O)n(C5(C(F)F)CC5)c4c3)c(Cl)c2)c(=O)[nH]c1=O] |
| 19 | 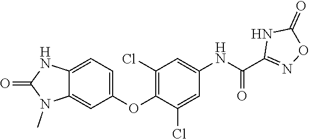 [SMILES: Cn1c(=O)[nH]c2ccc(Oc3c(Cl)cc(NC(=O)c4noc(=O)[nH]4)cc3Cl)cc21] |
| 20 | 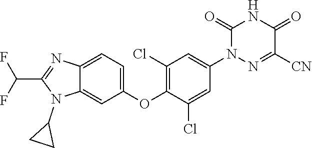 [SMILES: N#Cc1nn(-c2cc(Cl)c(Oc3ccc4nc(C(F)F)n(C5CC5)c4c3)c(Cl)c2)c(=O)[nH]c1=O] |
| 21 | 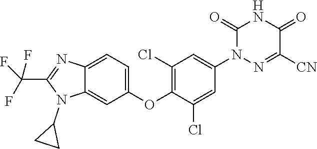 [SMILES: N#Cc1nn(-c2cc(Cl)c(Oc3ccc4nc(C(F)(F)F)n(C5CC5)c4c3)c(Cl)c2)c(=O)[nH]c1=O] |
| 22 | 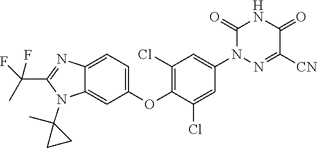 [SMILES: CC(F)(F)c1nc2ccc(Oc3c(Cl)cc(-n4nc(C#N)c(=O)[nH]c4=O)cc3Cl)cc2n1C1(C)CC1] |
| 23 | 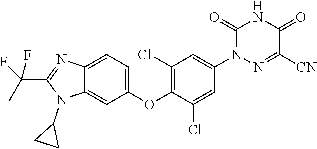 [SMILES: CC(F)(F)c1nc2ccc(Oc3c(Cl)cc(-n4nc(C#N)c(=O)[nH]c4=O)cc3Cl)cc2n1C1CC1] |
| 24 | 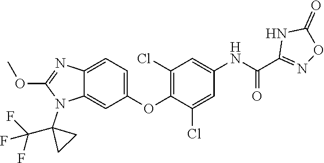 [SMILES: COc1nc2ccc(Oc3c(Cl)cc(NC(=O)c4noc(=O)[nH]4)cc3Cl)cc2n1C1(C(F)(F)F)CC1] |
| 25 | 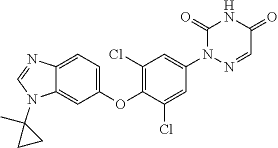 [SMILES: CC1(n2cnc3ccc(Oc4c(Cl)cc(-n5ncc(=O)[nH]c5=O)cc4Cl)cc32)CC1] |
| 26 | 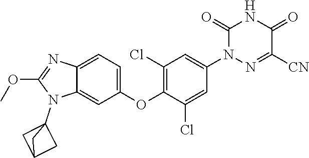 [SMILES: COc1nc2ccc(Oc3c(Cl)cc(-n4nc(C#N)c(=O)[nH]c4=O)cc3Cl)cc2n1C12CC(C1)C2] |
| 27 | 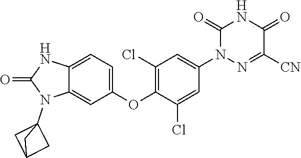 [SMILES: N#Cc1nn(-c2cc(Cl)c(Oc3ccc4[nH]c(=O)n(C56CC(C5)C6)c4c3)c(Cl)c2)c(=O)[nH]c1=O] |
| 28 | 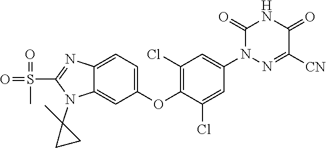 [SMILES: CC1(n2c(S(C)(=O)=O)nc3ccc(Oc4c(Cl)cc(-n5nc(C#N)c(=O)[nH]c5=O)cc4Cl)cc32)CC1] |
| 29 | 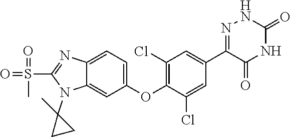 [SMILES: CC1(n2c(S(C)(=O)=O)nc3ccc(Oc4c(Cl)cc(-c5n[nH]c(=O)[nH]c5=O)cc4Cl)cc32)CC1] |
| 30 | 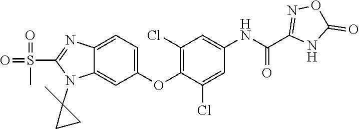 [SMILES: CC1(n2c(S(C)(=O)=O)nc3ccc(Oc4c(Cl)cc(NC(=O)c5noc(=O)[nH]5)cc4Cl)cc32)CC1] |
| 31 | 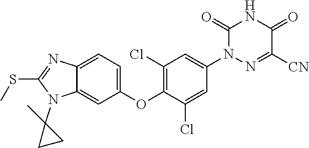 [SMILES: CSc1nc2ccc(Oc3c(Cl)cc(-n4nc(C#N)c(=O)[nH]c4=O)cc3Cl)cc2n1C1(C)CC1] |
| 32 | 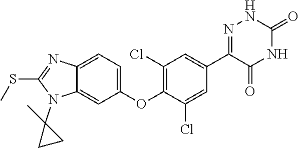 [SMILES: CSc1nc2ccc(Oc3c(Cl)cc(-c4n[nH]c(=O)[nH]c4=O)cc3Cl)cc2n1C1(C)CC1] |
| 33 | 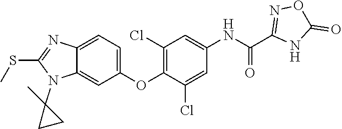 [SMILES: CSc1nc2ccc(Oc3c(Cl)cc(NC(=O)c4noc(=O)[nH]4)cc3Cl)cc2n1C1(C)CC1] |
| 34 | 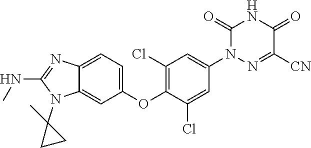 [SMILES: CNc1nc2ccc(Oc3c(Cl)cc(-n4nc(C#N)c(=O)[nH]c4=O)cc3Cl)cc2n1C1(C)CC1] |
| 35 | 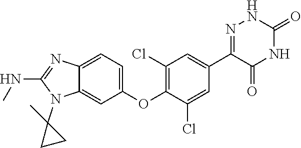 [SMILES: CNc1nc2ccc(Oc3c(Cl)cc(-c4n[nH]c(=O)[nH]c4=O)cc3Cl)cc2n1C1(C)CC1] |
| 36 | 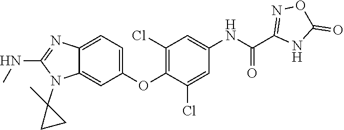 [SMILES: CNc1nc2ccc(Oc3c(Cl)cc(NC(=O)c4noc(=O)[nH]4)cc3Cl)cc2n1C1(C)CC1] |
| 37 | 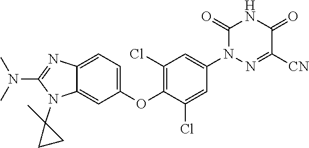 [SMILES: CN(C)c1nc2ccc(Oc3c(Cl)cc(-n4nc(C#N)c(=O)[nH]c4=O)cc3Cl)cc2n1C1(C)CC1] |
| 38 | 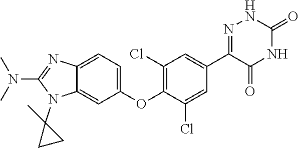 [SMILES: CN(C)c1nc2ccc(Oc3c(Cl)cc(-c4n[nH]c(=O)[nH]c4=O)cc3Cl)cc2n1C1(C)CC1] |
| 39 | 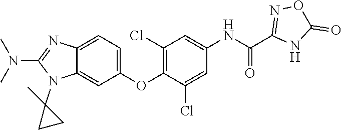 [SMILES: CN(C)c1nc2ccc(Oc3c(Cl)cc(NC(=O)c4noc(=O)[nH]4)cc3Cl)cc2n1C1(C)CC1] |
| 40 | 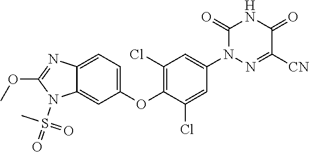 [SMILES: COc1nc2ccc(Oc3c(Cl)cc(-n4nc(C#N)c(=O)[nH]c4=O)cc3Cl)cc2n1S(C)(=O)=O] |
| 41 | 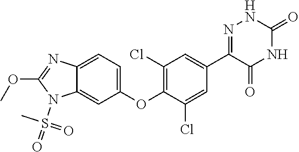 [SMILES: COc1nc2ccc(Oc3c(Cl)cc(-c4n[nH]c(=O)[nH]c4=O)cc3Cl)cc2n1S(C)(=O)=O] |
| 42 | 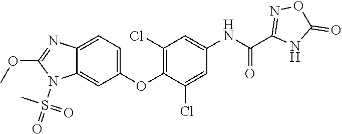 [SMILES: COc1nc2ccc(Oc3c(Cl)cc(NC(=O)c4noc(=O)[nH]4)cc3Cl)cc2n1S(C)(=O)=O] |

or a tautomer or stereoisomer of the aforesaid, or a pharmaceutically acceptable salt of each of the foregoing.

In some embodiments, provided herein is a compound selected from those listed in Table 1 or a pharmaceutically acceptable salt thereof. In some embodiments, provided herein is a compound selected from Examples 20-42, or a tautomer or stereoisomer of the aforesaid, or a pharmaceutically acceptable salt of each of the foregoing. In some embodiments, provided herein is a compound selected from Examples 20-42, or a pharmaceutically acceptable salt thereof.

The invention also includes all salts, such as pharmaceutically acceptable salts, of compounds referred to herein. The invention also includes any or all of the stereochemical forms, including any enantiomeric or diastereomeric forms, and any tautomers or other forms, such as N-oxides, solvates, or isotopomers, of the compounds described. Unless stereochemistry is explicitly indicated in a chemical structure or name, the structure or name is intended to embrace all possible stereoisomers of a compound depicted. In addition, where a specific stereochemical form is depicted, it is understood that other stereochemical forms are also embraced by the invention. All forms of the compounds are also embraced by the invention, such as crystalline or non-crystalline forms of the compounds. Compositions comprising a compound of the invention are also intended, such as a composition of substantially pure compound, including a specific stereochemical form thereof. Compositions comprising a mixture of compounds of the invention in any ratio are also embraced by the invention, including mixtures of two or more stereochemical forms of a compound of the invention in any ratio, such that racemic, non-racemic, enantioenriched and scalemic mixtures of a compound are embraced.

### Methods of Synthesis

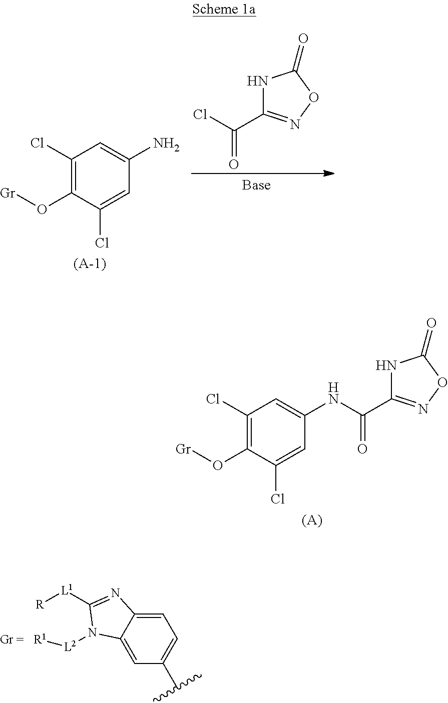

Scheme 1a shows a synthesis of compounds of general formula (A), wherein variables L¹, L², R and R¹ are as defined for the compound of formula (I). Amine derivatives of formula (A-1) can react with 5-oxo-4,5-dihydro-1,2,4-oxadiazole-3-carbonyl chloride in the presence of base to form compounds of formula (A).

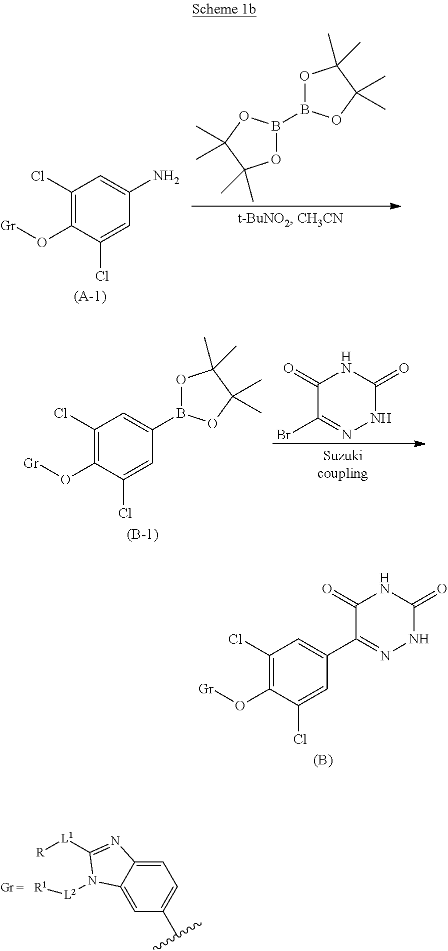

Scheme 1b outlines the general synthesis of compounds of formula (B), wherein variables L¹, L², R and R¹ are as defined for the compound of formula (I). Treatment of compounds of formula (A-1) with dioxaborolane 4,4,5,5-tetramethyl-2-(4,4,5,5-tetramethyl-1,3,2-dioxaborolan-2-yl)-1,3,2-dioxaborolane affords compounds of formula (B-1), which can then undergo Suzuki coupling with 6-bromo-1,2,4-triazine-3,5(2H,4H)-dione to form compounds of general formula (B).

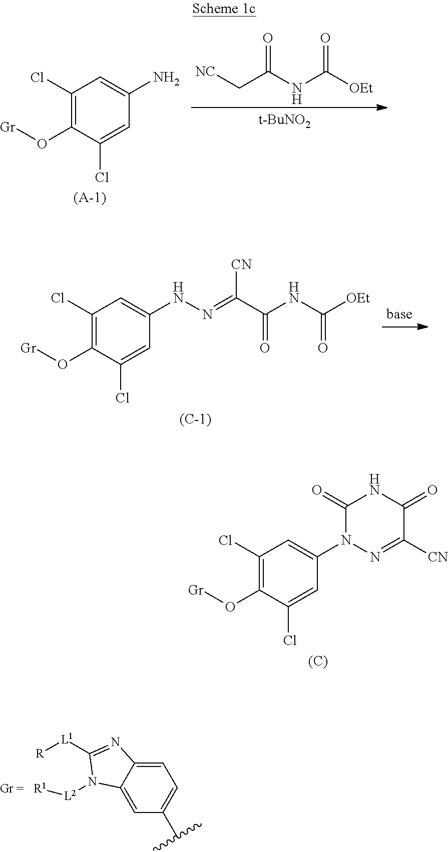

Scheme 1c shows a synthesis of compounds of general formula (C), wherein variables L¹, L², R and R¹ are as defined for the compound of formula (I). Reaction of compounds of formula (A-1) with ethyl (2-cyanoacetyl)carbamate affords intermediate compounds of formula (C-1), which can subsequently be treated with base to afford compounds of formula (C).

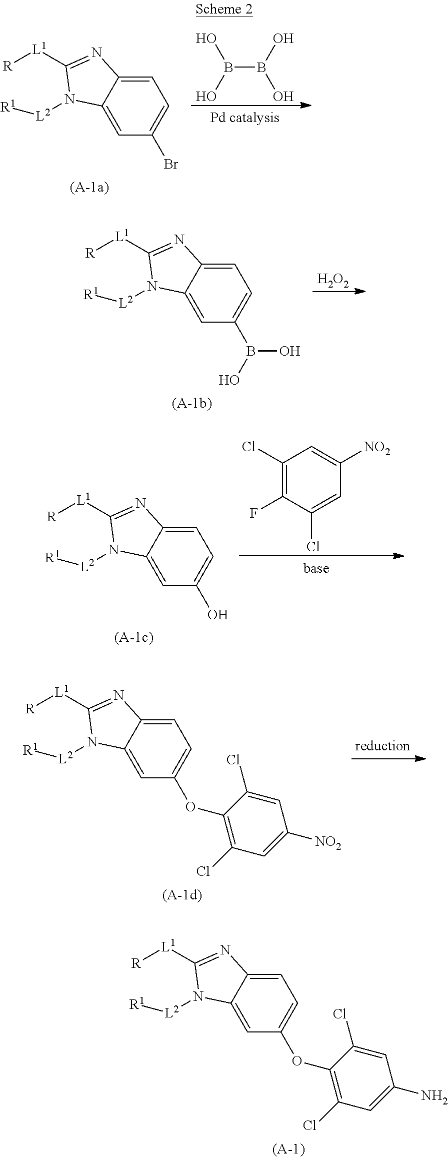

Scheme 2 shows a synthesis of compounds of general formula (A-1), wherein variables L¹, L², R and R¹ are as defined for the compound of formula (I), which are employed in the synthetic methods described herein and as outlined in Schemes 1a-1c. The bromide derivative (A-1a) can react with hypodiboric acid to form boronic acid derivative (A-1b), which can then be oxidized to form hydroxide (A-1c). Subsequent treatment of the compound of formula (A-1c) with 1,3-dichloro-2-fluoro-5-nitrobenzene and base affords the nitro derivative (A-1d), which can then be reduced to form a compound of formula (A-1).

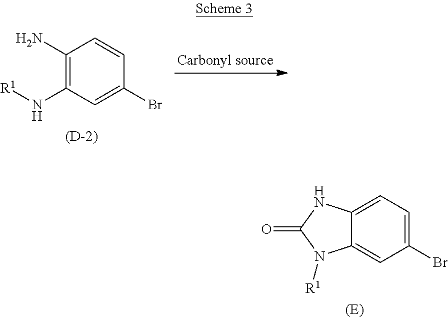

Scheme 3 outlines a synthesis of compounds of general formula (E), wherein variable R¹ is as defined for the compound of formula (I), which are used in the synthetic methods described herein for introducing the fused ring system containing ring B. Reaction of amine derivative (D-2) with a carbonyl source affords the compound of formula (E).

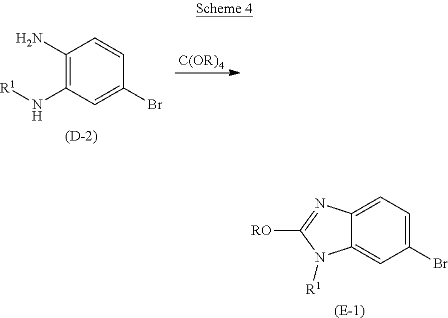

Scheme 4a outlines a procedure for preparing alkoxy derivatives of general formula (E-1), wherein variable R¹ is as defined for the compound of formula (I) and R is an alkyl group, which are used in the synthetic methods described herein for introducing the fused ring system containing ring B. Reaction of amine derivative (D-2) with C(OR)₄ as a carbonyl source affords the compound of formula (E-1). In some variations, R is methyl. In some embodiments, a compound of formula (E-1) is an intermediate in the preparation of a compound of formula (E), as provided in Scheme 3. In some embodiments, a compound of formula (E-1) can further react with an agent that cleaves C—O bonds in ethers (for example, BCl₃) to give a compound of formula (E), as provided in Scheme 3. In some embodiments, the compound of formula (E-1) is a compound of formula (A-1a), as provided in Scheme 2, and can react according to the general procedure outlined in Scheme 2, wherein the intermediate and product compounds retain the —OR functionality present in the compound of formula (E-1).

Synthesis of certain compounds provided herein are schematically illustrated above, and provided in the Examples section below. The variables listed in the schemes above are as defined for the compound of formula (I) or any variation, embodiments, or aspect thereof. Synthesis of other compounds provided herein will be apparent to the skilled artisan based on the guidance provided herein and based on synthetic methods well known to the skilled artisan.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization, and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates of a compound provided herein or a pharmaceutically acceptable salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol.

It is understood that the synthetic process disclosed here may be modified to arrive at various compounds of the invention by selection of appropriate reagents and starting materials. It is also understood that where protection of certain active or incompatible groups (e.g., an amine or a carboxylic acid) is required, the formulae in e.g., the scheme(s) provided here intend and include compounds where such active or incompatible groups are in appropriate protected forms. For a general description of protecting groups and their use, see P. G. M. Wuts and T. W. Greene, Greene's Protective Groups in Organic Synthesis 4^{th} edition, Wiley-Interscience, New York, 2006.

### Pharmaceutical Compositions and Formulations

Pharmaceutical compositions of any of the compounds detailed herein are embraced by this invention. Thus, the invention includes pharmaceutical compositions comprising a compound of the invention or a pharmaceutically acceptable salt thereof and a pharmaceutically acceptable carrier or excipient. In some embodiments, the pharmaceutically acceptable salt is an acid addition salt, such as a salt formed with an inorganic or organic acid. Pharmaceutical compositions according to the invention may take a form suitable for oral, buccal, parenteral, nasal, topical or rectal administration or a form suitable for administration by inhalation.

A compound as detailed herein may in some embodiments be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound selected from a compound of Table 1 intends a composition that contains no more than 35% impurity, wherein the impurity denotes a compound other than the compound or a salt thereof. In one variation, a composition of substantially pure compound or a salt thereof is provided wherein the composition contains no more than 25% impurity. In another variation, a composition of substantially pure compound or a salt thereof is provided wherein the composition contains no more than 20% impurity. In still another variation, a composition of substantially pure compound or a salt thereof is provided wherein the composition contains no more than 10% impurity. In a further variation, a composition of substantially pure compound or a salt thereof is provided wherein the composition contains no more than 5% impurity. In another variation, a composition of substantially pure compound or a salt thereof is provided wherein the composition contains no more than 3% impurity. In still another variation, a composition of substantially pure compound or a salt thereof is provided wherein the composition contains no more than 1% impurity. In a further variation, a composition of substantially pure compound or a salt thereof is provided wherein the composition contains no more than 0.5% impurity. In yet other variations, a composition of substantially pure compound means that the composition contains no more than 15%, or preferably no more than 10%, or more preferably no more than 5%, or even more preferably no more than 3%, and most preferably no more than 1% impurity, which impurity may be the compound in a different stereochemical form. For instance, and without limitation, a composition of substantially pure (S) compound means that the composition contains no more than 15%, or no more than 10%, or no more than 5%, or no more than 3%, or no more than 1% of the (R) form of the compound.

In one variation, the compounds herein are synthetic compounds prepared for administration to an individual such as a human. In another variation, compositions are provided containing a compound in substantially pure form. In another variation, the invention embraces pharmaceutical compositions comprising a compound detailed herein and a pharmaceutically acceptable carrier or excipient. In another variation, methods of administering a compound are provided. The purified forms, pharmaceutical compositions and methods of administering the compounds are suitable for any compound or form thereof detailed herein.

The compound may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein can be used in the preparation of a formulation, such as a pharmaceutical formulation, by combining the compound or compounds as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21^{st} ed. (2005), which is incorporated herein by reference.

Compounds as described herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid polyols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described.

Compositions comprising a compound provided herein, or a pharmaceutically acceptable salt thereof, are also described. In one variation, the composition comprises a compound and a pharmaceutically acceptable carrier or excipient. In another variation, a composition of substantially pure compound is provided.

### Methods of Use/Treatments

Compounds and compositions detailed herein, such as a pharmaceutical composition containing a compound of any formula provided herein, or a pharmaceutically acceptable salt thereof, and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

In some embodiments, provided herein is a method of agonizing thyroid hormone receptor beta (THR beta) comprising contacting either an effective amount of a compound provided herein, or a pharmaceutically acceptable salt thereof, or an effective amount of a pharmaceutical composition provided herein, with the THR beta.

In some embodiments, provided herein is a method of treating a disorder, which is ameliorated by activation of THR beta, in a patient, comprising administering to the patient in need thereof a therapeutically effective amount of a compound provided herein, or a pharmaceutically acceptable salt thereof, or a therapeutically effective amount of a composition provided herein.

Methods of treating a disorder ameliorated by activation of THR beta, including without limitation non-alcoholic fatty liver disease, non-alcoholic steatohepatitis, and symptoms and manifestations of each thereof are well known to the skilled artisan and can be adapted to treating such a disorder with a compound, or a pharmaceutically acceptable salt thereof, or composition provided herein.

In some embodiments, provided herein is a method of agonizing thyroid hormone receptor beta (THR beta) comprising contacting either an effective amount of a compound provided herein, or a salt thereof, such as a pharmaceutically acceptable salt thereof, or an effective amount of a pharmaceutical composition provided herein, with the THR beta. In some embodiments, provided herein is a method of selectively agonizing THR beta over THR alpha comprising contacting either an effective amount of a compound provided herein, or a pharmaceutically acceptable salt thereof, or an effective amount of a pharmaceutical composition provided herein, with the THR beta. In one such aspect, the method selectively agonizes THR beta over THR alpha by at least 2-, 3-, 4-, 5-, 6-, 7-, 8-, 9-, 10-, 15-, 20-, 25-, 30-, 35-, 40-, 45-, 50-, 55-, 60-, 65-, 70-, 75-, 80-, 85-, 90-, 95-, or 100-fold. In any such embodiment, in some embodiments selectivity is assessed via a biochemical assay, such as the TR-FRET assay described in Example B 1. In any such embodiment, in another aspect selectivity is assessed via a biochemical assay, such as the RXR heterodimer assay described in Example B2.

In some embodiments, provided herein is a method of treating a disease or disorder that is ameliorated by activation of THR beta in a patient in need thereof, comprising administering to the patient a therapeutically effective amount of a compound provided herein, or a pharmaceutically acceptable salt thereof, or a therapeutically effective amount of a composition provided herein. In some embodiments, the disease or disorder is a liver disease or disorder. In some embodiments, provided herein is a method of treating a disease or disorder of the liver associated with sub-optimal THR beta agonism in a patient in need thereof, comprising administering to the patient a compound of formula (I), or a pharmaceutically acceptable salt thereof, wherein the compound selectively agonizes THR beta over THR alpha.

In some embodiments, provided herein is a method of treating non-alcoholic fatty liver disease in a patient in need thereof, comprising administering to the patient a therapeutically effective amount of a compound provided herein, or a pharmaceutically acceptable salt thereof, or a therapeutically effective amount of a composition provided herein. In some embodiments, provided herein is a method of treating non-alcoholic steatohepatitis (NASH) in a patient in need thereof, comprising administering to the patient a therapeutically effective amount of a compound provided herein, or a pharmaceutically acceptable salt thereof, or a therapeutically effective amount of a composition provided herein. In some embodiments, provided herein is a method of treating metabolic syndrome in a patient in need thereof, comprising administering to the patient a therapeutically effective amount of a compound provided herein, or a pharmaceutically acceptable salt thereof, or a therapeutically effective amount of a composition provided herein. In some embodiments, provided herein is a method of treating dyslipidemia in a patient in need thereof, comprising administering to the patient a therapeutically effective amount of a compound provided herein, or a pharmaceutically acceptable salt thereof, or a therapeutically effective amount of a composition provided herein. In some embodiments, provided herein is a method of treating hypertriglyceridemia in a patient in need thereof, comprising administering to the patient a therapeutically effective amount of a compound provided herein, or a pharmaceutically acceptable salt thereof, or a therapeutically effective amount of a composition provided herein. In some embodiments, provided herein is a method of treating hypercholesterolemia in a patient in need thereof, comprising administering to the patient a therapeutically effective amount of a compound provided herein, or a pharmaceutically acceptable salt thereof, or a therapeutically effective amount of a composition provided herein.

In any of the embodiments described herein, a patient having a disease or disorder associated with THR beta agonism may include, but is not limited to, a patient with an underlying hypothyroid disorder.

In another aspect is provided a method of delaying the onset and/or development of a disease or disorder that is ameliorated by activation of THR beta in a patient (such as a human) who is at risk for developing the disease or disorder. It is appreciated that delayed development may encompass prevention in the event the individual does not develop the disease or disorder. An individual at risk of developing a disease or disorder that is ameliorated by activation of THR beta in some embodiments has one or more risk factors for developing the disease or disorder, such as age, increased waist circumference, high body to mass index or the presence of an associated comorbidity.

In some embodiments, provided herein is a method of delaying the onset and/or development of non-alcoholic fatty liver disease in a patient in need thereof, comprising administering to the patient a therapeutically effective amount of a compound provided herein, or a pharmaceutically acceptable salt thereof, or a therapeutically effective amount of a composition provided herein. In some embodiments, provided herein is a method of delaying the onset and/or development of non-alcoholic steatohepatitis (NASH) in a patient in need thereof, comprising administering to the patient a therapeutically effective amount of a compound provided herein, or a pharmaceutically acceptable salt thereof, or a therapeutically effective amount of a composition provided herein. In some embodiments, provided herein is a method of delaying the onset and/or development of metabolic syndrome in a patient in need thereof, comprising administering to the patient a therapeutically effective amount of a compound provided herein, or a pharmaceutically acceptable salt thereof, or a therapeutically effective amount of a composition provided herein. In some embodiments, provided herein is a method of delaying the onset and/or development of dyslipidemia in a patient in need thereof, comprising administering to the patient a therapeutically effective amount of a compound provided herein, or a pharmaceutically acceptable salt thereof, or a therapeutically effective amount of a composition provided herein. In some embodiments, provided herein is a method of delaying the onset and/or development of hypertriglyceridemia in a patient in need thereof, comprising administering to the patient a therapeutically effective amount of a compound provided herein, or a pharmaceutically acceptable salt thereof, or a therapeutically effective amount of a composition provided herein. In some embodiments, provided herein is a method of delaying the onset and/or development of hypercholesterolemia in a patient in need thereof, comprising administering to the patient a therapeutically effective amount of a compound provided herein, or a pharmaceutically acceptable salt thereof, or a therapeutically effective amount of a composition provided herein.

In some embodiments, provided herein is a compound of formula (I) or any variation thereof, or a pharmaceutically acceptable salt thereof, for use in therapy. In some embodiments, provided herein is a compound of formula (I) or any variation thereof, or a pharmaceutically acceptable salt thereof or pharmaceutical composition comprising such compound or a pharmaceutically acceptable salt thereof, for use in the treatment of non-alcoholic fatty liver disease. In some embodiments, provided herein is a compound of formula (I) or any variation thereof, or a pharmaceutically acceptable salt thereof or pharmaceutical composition comprising such compound or a pharmaceutically acceptable salt thereof, for use in the treatment of non-alcoholic steatohepatitis (NASH). In some embodiments, provided is a compound of formula (I) or any variation thereof, or a pharmaceutically acceptable salt thereof, or a pharmaceutical composition comprising such compound or a pharmaceutically acceptable salt thereof, for use in the treatment of metabolic syndrome. In some embodiments, provided is a compound of formula (I) or any variation thereof, or a pharmaceutically acceptable salt thereof, or a pharmaceutical composition comprising such compound or a pharmaceutically acceptable salt thereof, for use in the treatment of dyslipidemia. In some embodiments, provided is a compound of formula (I) or any variation thereof, or a pharmaceutically acceptable salt thereof, or a pharmaceutical composition comprising such compound or a pharmaceutically acceptable salt thereof, for use in the treatment of hypertriglyceridemia. In some embodiments, provided is a compound of formula (I) or any variation thereof, or a pharmaceutically acceptable salt thereof, or a pharmaceutical composition comprising such compound or a pharmaceutically acceptable salt thereof, for use in the treatment of hypercholesterolemia.

In another embodiment, provided herein is a compound of formula (I) or any variation thereof, or a pharmaceutically acceptable salt thereof, for use in the manufacture of a medicament for the treatment of non-alcoholic fatty liver disease. In another embodiment, provided herein is a compound of formula (I) or any variation thereof, or a pharmaceutically acceptable salt thereof, for use in the manufacture of a medicament for the treatment of non-alcoholic steatohepatitis (NASH). In another embodiment, provided herein is a compound of formula (I) or any variation thereof, or a pharmaceutically acceptable salt thereof, for use in the manufacture of a medicament for the treatment of metabolic syndrome. In some embodiments, the medicament is for the treatment of dyslipidemia. In some embodiments, the medicament is for the treatment of hypertriglyceridemia. In some embodiments, the medicament is for the treatment of dyslipidemia. In some embodiments, the medicament is for the treatment of hypercholesterolemia.

In some embodiments of a method disclosed herein, the method comprises admistering a therapeutically effect amount of a compound of formula (I), or a tautomer or stereoisomer thereof, or a pharmaceutically acceptable salt of any of the foregoing, and the therapeutically effect amount is less than about 5 mg/kg/day, about 4 mg/kg/day, about 3 mg/kg/day, about 2 mg/kg/day, about 1 mg/kg/day, about 0.5 mg/kg/day, or about 0.1 mg/kg/day. In some embodiments, the method comprises admistering a therapeutically effect amount of a compound of formula (I) such as formula (I-3), or a tautomer or stereoisomer thereof, or a pharmaceutically acceptable salt of any of the foregoing, wherein R is H, and the therapeutically effect amount is less than about 5 mg/kg/day, about 4 mg/kg/day, about 3 mg/kg/day, about 2 mg/kg/day, about 1 mg/kg/day, about 0.5 mg/kg/day, or about 0.1 mg/kg/day. In some embodiments, the method comprises admistering a therapeutically effect amount of

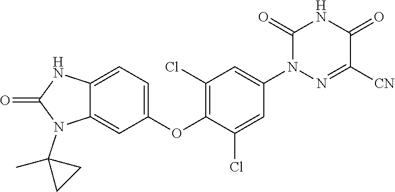 [SMILES: CC1(n2c(=O)[nH]c3ccc(Oc4c(Cl)cc(-n5nc(C#N)c(=O)[nH]c5=O)cc4Cl)cc32)CC1]

or a pharmaceutically acceptable salt thereof, and the therapeutically effect amount is less than about 5 mg/kg/day, about 4 mg/kg/day, about 3 mg/kg/day, about 2 mg/kg/day, about 1 mg/kg/day, about 0.5 mg/kg/day, or about 0.1 mg/kg/day. In some embodiments, the method comprises admistering a therapeutically effect amount of a compound of formula (I) such as formula (I-3), or a tautomer or stereoisomer thereof, or a pharmaceutically acceptable salt of any of the foregoing, wherein R is C₁-C₆ alkyl, and the therapeutically effect amount is less than about 5 mg/kg/day, about 4 mg/kg/day, about 3 mg/kg/day, about 2 mg/kg/day, about 1 mg/kg/day, about 0.5 mg/kg/day, or about 0.1 mg/kg/day. In some embodiments, the method comprises admistering a therapeutically effect amount of a compound of formula (I) such as formula (I-3), or a tautomer or stereoisomer thereof, or a pharmaceutically acceptable salt of any of the foregoing, wherein R is methyl, and the therapeutically effect amount is less than about 5 mg/kg/day, about 4 mg/kg/day, about 3 mg/kg/day, about 2 mg/kg/day, about 1 mg/kg/day, about 0.5 mg/kg/day, or about 0.1 mg/kg/day. In some embodiments, the method comprises admistering a therapeutically effect amount of

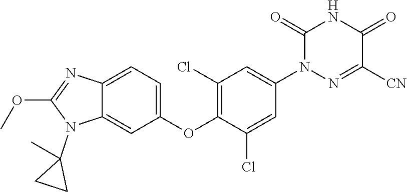 [SMILES: COc1nc2ccc(Oc3c(Cl)cc(-n4nc(C#N)c(=O)[nH]c4=O)cc3Cl)cc2n1C1(C)CC1]

or a pharmaceutically acceptable salt thereof and the therapeutically effect amount is less than about 5 mg/kg/day, about 4 mg/kg/day, about 3 mg/kg/day, about 2 mg/kg/day, about 1 mg/kg/day, about 0.5 mg/kg/day, or about 0.1 mg/kg/day.

In some embodiments of a method disclosed herein, the method comprises admistering a therapeutically effect amount of a compound of formula (I), or a tautomer or stereoisomer thereof, or a pharmaceutically acceptable salt of any of the foregoing, and the therapeutically effect amount is less than about 100 mg/day, 90 mg/day, 80 mg/day, 70 mg/day, 60 mg/day, 50 mg/day, 40 mg/day, 30 mg/day, 20 mg/day, 10 mg/day, or 5 mg/day. In some embodiments, the method comprises admistering a therapeutically effect amount of a compound of formula (I) such as formula (I-3), or a tautomer or stereoisomer thereof, or a pharmaceutically acceptable salt of any of the foregoing, wherein R is H, and the therapeutically effect amount is less than about 100 mg/day, 90 mg/day, 80 mg/day, 70 mg/day, 60 mg/day, 50 mg/day, 40 mg/day, 30 mg/day, 20 mg/day, 10 mg/day, or 5 mg/day. In some embodiments, the method comprises admistering a therapeutically effect amount of

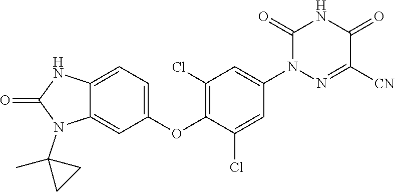 [SMILES: CC1(n2c(=O)[nH]c3ccc(Oc4c(Cl)cc(-n5nc(C#N)c(=O)[nH]c5=O)cc4Cl)cc32)CC1]

or a pharmaceutically acceptable salt thereof, and the therapeutically effect amount is less than about 100 mg/day, 90 mg/day, 80 mg/day, 70 mg/day, 60 mg/day, 50 mg/day, 40 mg/day, 30 mg/day, 20 mg/day, 10 mg/day, or 5 mg/day. In some embodiments, the method comprises admistering a therapeutically effect amount of a compound of formula (I) such as formula (I-3), or a tautomer or stereoisomer thereof, or a pharmaceutically acceptable salt of any of the foregoing, wherein R is C₁-C₆ alkyl, and the therapeutically effect amount is less than about 100 mg/day, 90 mg/day, 80 mg/day, 70 mg/day, 60 mg/day, 50 mg/day, 40 mg/day, 30 mg/day, 20 mg/day, 10 mg/day, or 5 mg/day. In some embodiments, the method comprises admistering a therapeutically effect amount of a compound of formula (I) such as formula (I-3), or a tautomer or stereoisomer thereof, or a pharmaceutically acceptable salt of any of the foregoing, wherein R is methyl, and the therapeutically effect amount is less than about 100 mg/day, 90 mg/day, 80 mg/day, 70 mg/day, 60 mg/day, 50 mg/day, 40 mg/day, 30 mg/day, 20 mg/day, 10 mg/day, or 5 mg/day. In some embodiments, the method comprises admistering a therapeutically effect amount of

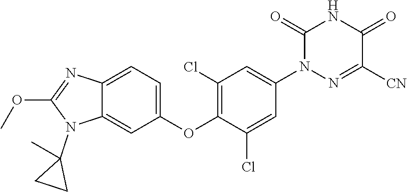 [SMILES: COc1nc2ccc(Oc3c(Cl)cc(-n4nc(C#N)c(=O)[nH]c4=O)cc3Cl)cc2n1C1(C)CC1]

or a pharmaceutically acceptable salt thereof and the therapeutically effect amount is less than about 100 mg/day, 90 mg/day, 80 mg/day, 70 mg/day, 60 mg/day, 50 mg/day, 40 mg/day, 30 mg/day, 20 mg/day, 10 mg/day, or 5 mg/day.

In some embodiments of a method disclosed herein, the method comprises admistering a therapeutically effect amount of a compound of formula (I), or a tautomer or stereoisomer thereof, or a pharmaceutically acceptable salt of any of the foregoing, and the maximum blood, serum, or plasma level of a compound of formula (I) during the treatment period is less than about 200 ng/ml, 190 ng/ml, 180 ng/ml, 170 ng/ml, 160 ng/ml, 150 ng/ml, 140 ng/ml, 130 ng/ml, 120 ng/ml, 110 ng/ml, 100 ng/ml, 90 ng/ml, 80 ng/ml, 70 ng/ml, 60 ng/ml, 50 ng/ml, 40 ng/ml, 30 ng/ml, 20 ng/ml, 10 ng/ml, or 5 ng/ml. In some embodiments, the method comprises admistering a therapeutically effect amount of a compound of formula (I) such as formula (I-3), or a tautomer or stereoisomer thereof, or a pharmaceutically acceptable salt of any of the foregoing, wherein R is H, and the maximum blood, serum, or plasma level of a compound of formula (I) during the treatment period is less than about 200 ng/ml, 190 ng/ml, 180 ng/ml, 170 ng/ml, 160 ng/ml, 150 ng/ml, 140 ng/ml, 130 ng/ml, 120 ng/ml, 110 ng/ml, 100 ng/ml, 90 ng/ml, 80 ng/ml, 70 ng/ml, 60 ng/ml, 50 ng/ml, 40 ng/ml, 30 ng/ml, 20 ng/ml, 10 ng/ml, or 5 ng/ml. In some embodiments, the method comprises admistering a therapeutically effect amount of

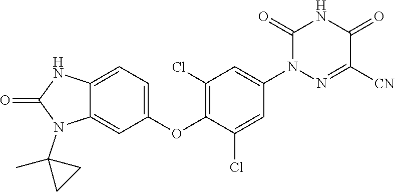 [SMILES: CC1(n2c(=O)[nH]c3ccc(Oc4c(Cl)cc(-n5nc(C#N)c(=O)[nH]c5=O)cc4Cl)cc32)CC1]

or a pharmaceutically acceptable salt thereof, and the maximum blood, serum, or plasma level of the compound during the treatment period is less than about 200 ng/ml, 190 ng/ml, 180 ng/ml, 170 ng/ml, 160 ng/ml, 150 ng/ml, 140 ng/ml, 130 ng/ml, 120 ng/ml, 110 ng/ml, 100 ng/ml, 90 ng/ml, 80 ng/ml, 70 ng/ml, 60 ng/ml, 50 ng/ml, 40 ng/ml, 30 ng/ml, 20 ng/ml, 10 ng/ml, or 5 ng/ml.

In some embodiments of a method disclosed herein, the method comprises admistering a therapeutically effect amount of a compound of formula (I), or a tautomer or stereoisomer thereof, or a pharmaceutically acceptable salt of any of the foregoing, and the blood, serum, or plasma AUC of a compound of formula (I) after a single dose is less than about 3,000 ng·h/mL, about 2,500 ng·h/mL, about 2,000 ng·h/mL, about 1,500 ng·h/mL, about 1,000 ng·h/mL, about 1,500 ng·h/mL, or about 500 ng·h/mL. In some embodiments, the method comprises admistering a therapeutically effect amount of a compound of formula (I) such as formula (I-3), or a tautomer or stereoisomer thereof, or a pharmaceutically acceptable salt of any of the foregoing, wherein R is H, and the blood, serum, or plasma AUC of a compound of formula (I) after a single dose is less than about 3,000 ng·h/mL, about 2,500 ng·h/mL, about 2,000 ng·h/mL, about 1,500 ng·h/mL, about 1,000 ng·h/mL, about 1,500 ng·h/mL, or about 500 ng·h/mL. In some embodiments, the method comprises admistering a therapeutically effect amount of

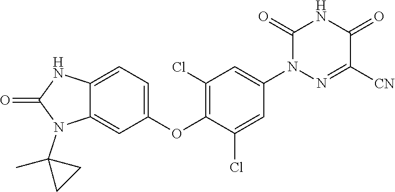 [SMILES: CC1(n2c(=O)[nH]c3ccc(Oc4c(Cl)cc(-n5nc(C#N)c(=O)[nH]c5=O)cc4Cl)cc32)CC1]

or a pharmaceutically acceptable salt thereof, and the blood, serum, or plasma AUC of the compound after a single dose is less than about 3,000 ng·h/mL, about 2,500 ng·h/mL, about 2,000 ng·h/mL, about 1,500 ng·h/mL, about 1,000 ng·h/mL, about 1,500 ng·h/mL, or about 500 ng·h/mL.

In some embodiments of a method disclosed herein, the method comprises admistering a therapeutically effect amount of a compound of formula (I), or a tautomer or stereoisomer thereof, or a pharmaceutically acceptable salt of any of the foregoing, and the ratio of the levl of a compound of formula (I) in liver to blood, serum, or plasma level of a compound of formula (I) is more than about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, or about 50. In some embodiments, the method comprises admistering a therapeutically effect amount of a compound of formula (I) such as formula (I-3), or a tautomer or stereoisomer thereof, or a pharmaceutically acceptable salt of any of the foregoing, wherein R is H, and the ratio of the levl of a compound of formula (I) in liver to blood, serum, or plasma level of a compound of formula (I) is more than about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, or about 50. In some embodiments, the method comprises admistering a therapeutically effect amount of

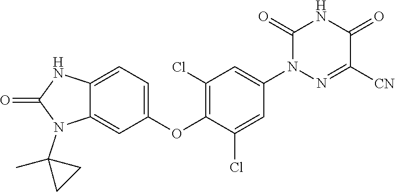 [SMILES: CC1(n2c(=O)[nH]c3ccc(Oc4c(Cl)cc(-n5nc(C#N)c(=O)[nH]c5=O)cc4Cl)cc32)CC1]

or a pharmaceutically acceptable salt thereof, and the ratio of the levl of the compound in liver to blood, serum, or plasma level of the compound is more than about 2, about 3, about 4, about 5, about 6, about 7, about 8, about 9, about 10, about 11, about 12, about 13, about 14, about 15, about 16, about 17, about 18, about 19, about 20, or about 25.

In some embodiments of a method disclosed herein, the method comprises admistering a therapeutically effect amount of a compound of formula (I), or a tautomer or stereoisomer thereof, or a pharmaceutically acceptable salt of any of the foregoing, and the ratio of the levl of a compound of formula (I) in liver to the level of a compound of formula (I) in an organ that is not liver is more than about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, or about 100. In some embodiments, the method comprises admistering a therapeutically effect amount of a compound of formula (I) such as formula (I-3), or a tautomer or stereoisomer thereof, or a pharmaceutically acceptable salt of any of the foregoing, wherein R is H, and the ratio of the levl of a compound of formula (I) in liver to the level of a compound of formula (I) in an organ that is not liver is more than about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, or about 100. In some embodiments, the method comprises admistering a therapeutically effect amount of

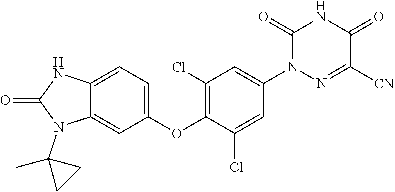 [SMILES: CC1(n2c(=O)[nH]c3ccc(Oc4c(Cl)cc(-n5nc(C#N)c(=O)[nH]c5=O)cc4Cl)cc32)CC1]

or a pharmaceutically acceptable salt thereof, and the ratio of the levl of the compound in liver to the level of the compound in an organ that is not liver is more than about 2, about 3, about 4, about 5, about 6, about 7, about 8, about 9, or about 10.

In some embodiments of a method disclosed herein, the method comprises admistering a therapeutically effect amount of a compound of formula (I), or a tautomer or stereoisomer thereof, or a pharmaceutically acceptable salt of any of the foregoing, and the method reduces the circulation levels (e.g., blood, serum, or plasma level) of one or more elements of the Hypothalamic-pituitary-thyroid (HPT) axis by less than about 10%, about 9%, about 8%, about 7%, about 6%, about 5%, about 4%, about 3%, about 2%, or about 1%. In some embodiments, the method comprises admistering a therapeutically effect amount of a compound of formula (I), or a tautomer or stereoisomer thereof, or a pharmaceutically acceptable salt of any of the foregoing, wherein R is H, and the method reduces the circulation levels (e.g., blood, serum, or plasma level) of one or more elements of the Hypothalamic-pituitary-thyroid (HPT) axis by less than about 10%, about 9%, about 8%, about 7%, about 6%, about 5%, about 4%, about 3%, about 2%, or about 1%. In some embodiments, the method comprises admistering a therapeutically effect amount of

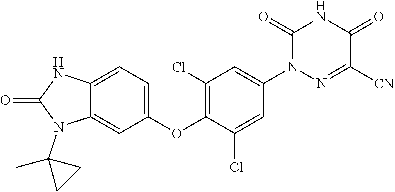 [SMILES: CC1(n2c(=O)[nH]c3ccc(Oc4c(Cl)cc(-n5nc(C#N)c(=O)[nH]c5=O)cc4Cl)cc32)CC1]

or a pharmaceutically acceptable salt thereof, and the method reduces the circulation levels (e.g., blood, serum, or plasma level) of one or more elements of the Hypothalamic-pituitary-thyroid (HPT) axis by less than about 10%, about 9%, about 8%, about 7%, about 6%, about 5%, about 4%, about 3%, about 2%, or about 1%. Examples of elements of the HPT axis include, without limitation, Triiodothyronine (T3), Thyroxine (T4), iodothyronines, thyrotropin-releasing hormone (TRH), and thyroid-stimulating hormone (TSH). In some emobidments, a method disclosed herein reduces the circulation level (e.g., blood, serum, or plasma level) of T3 by by less than about 10%, about 9%, about 8%, about 7%, about 6%, about 5%, about 4%, about 3%, about 2%, or about 1%. In some emobidments, a method disclosed herein reduces the circulation level (e.g., blood, serum, or plasma level) of T4 by by less than about 10%, about 9%, about 8%, about 7%, about 6%, about 5%, about 4%, about 3%, about 2%, or about 1%. In some emobidments, a method disclosed herein reduces the circulation level (e.g., blood, serum, or plasma level) of iodothyronines by by less than about 50%, about 45%, about 40%, about 35%, about 30%, about 25%, about 20%, about 15%, about 10%, about 9%, about 8%, about 7%, about 6%, about 5%, about 4%, about 3%, about 2%, or about 1%. In some emobidments, a method disclosed herein reduces the circulation level (e.g., blood, serum, or plasma level) of TRH by by less than about 50%, about 45%, about 40%, about 35%, about 30%, about 25%, about 20%, about 15%, about 10%, about 9%, about 8%, about 7%, about 6%, about 5%, about 4%, about 3%, about 2%, or about 1%. In some emobidments, a method disclosed herein reduces the circulation level (e.g., blood, serum, or plasma level) of TSH by by less than about 50%, about 45%, about 40%, about 35%, about 30%, about 25%, about 20%, about 15%, about 10%, about 9%, about 8%, about 7%, about 6%, about 5%, about 4%, about 3%, about 2%, or about 1%.

### Dosing and Method of Administration

The dose of a compound described herein, or a stereoisomer, tautomer, solvate, or salt thereof, administered to an individual (such as a human) may vary with the particular compound or salt thereof, the method of administration, and the particular disease or disorder, such as non-alcoholic fatty liver disease, non-alcoholic steatohepatitis (NASH), metabolic syndrome, hypertriglyceridemia, dyslipidemia, or hypercholesterolemia, being treated. In some embodiments, the amount of the compound, or a stereoisomer, tautomer, solvate, or salt thereof, is a therapeutically effective amount.

The compounds provided herein or a salt thereof may be administered to an individual via various routes, including, e.g., intravenous, intramuscular, subcutaneous, oral, and transdermal.

Any of the methods provided herein may in some embodiments comprise administering to an individual a pharmaceutical composition that contains an effective amount of a compound provided herein, or a stereoisomer, tautomer, solvate, or salt thereof, and a pharmaceutically acceptable excipient.

A compound or composition provided herein may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein, or a pharmaceutically acceptable salt thereof, together with any of the dosages described herein.

### Articles of Manufacture and Kits

The present disclosure further provides articles of manufacture comprising a compound described herein or a salt thereof, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises one or more compounds described herein, or a pharmaceutically acceptable salt thereof, or a composition comprising a compound described herein. The kits may employ any of the compounds disclosed herein or a pharmaceutically acceptable salt thereof. In one variation, the kit employs a compound described herein or pharmaceutically acceptable salt thereof. The kits may be used for any one or more of the uses described herein, and, accordingly, may contain instructions for the treatment of any disease or described herein, for example for the treatment of non-alcoholic steatohepatitis (NASH).

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein, or a pharmaceutically acceptable salt thereof, and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### EXAMPLES

It is understood that the present disclosure has been made only by way of example, and that numerous changes in the combination and arrangement of parts can be resorted to by those skilled in the art without departing from the spirit and scope of present disclosure.

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

The following abbreviations may be relevant for the application.

### Abbreviations

Ac: acetyl

ACN or MeCN: acetonitrile

BAST: bis(2-methoxyethyl)aminosulfurtrifluoride

BINAP: 2,2′-bis(diphenylphosphino)-1,1′-binaphthyl

BPD: bispinacolatodiboron

Boc: tertiarybutyloxycarbonyl

Bu: butyl

cataCXium A-Pd-G2: chloro [(di(1-adamantyl)-N-butylphosphine)-2-(2-aminobiphenyl)]palladium(II)

DBA: dibenzylideneacetone

DCM: dichloromethane

DIEA or DIPEA: N,N-diisopropylethylamine

DMA: dimethylacetamide

DMAP: dimethylaminopyridine

DMF: dimethylformamide

DMF-DMA: dimethylformamide dimethylacetal

DMSO: dimethylsulfoxide

DPPA: diphenylphosphoryl azide

DSC: disuccinimidylcarbonate

Et: ethyl

FA: formic acid

MBTE: methyl tert-butyl ether

Me: methyl

NIS: N-iodosuccinimide

Pd(dba)₂: bis(dibenzylideneacetone)palladium(0)

Pr: propyl

Py or Pyr: pyridine

rt: room temperature

sat: saturated

SEMCl: 2-(trimethylsilyl)ethoxymethyl chloride

SFC: supercritical fluid chromatography

TEA: triethylamine

TFA: trifluoroacetic acid

THF: tetrahydrofuran

Tol: toluene

XPhos: 2-dicyclohexylphosphino-2′,4′,6′-triisopropylbiphenyl

t-Bu Xphos: 2-di-tert-butylphosphino-2′,4′,6′-triisopropylbiphenyl

### Synthetic Examples

### Example 1

### N-(3,5-dichloro-4-((3-cyclopropyl-2-oxo-2,3-dihydro-1H-benzo[d]imidazol-5-yl)oxy)phenyl)-5-oxo-4,5-dihydro-1,2,4-oxadiazole-3-carboxamide

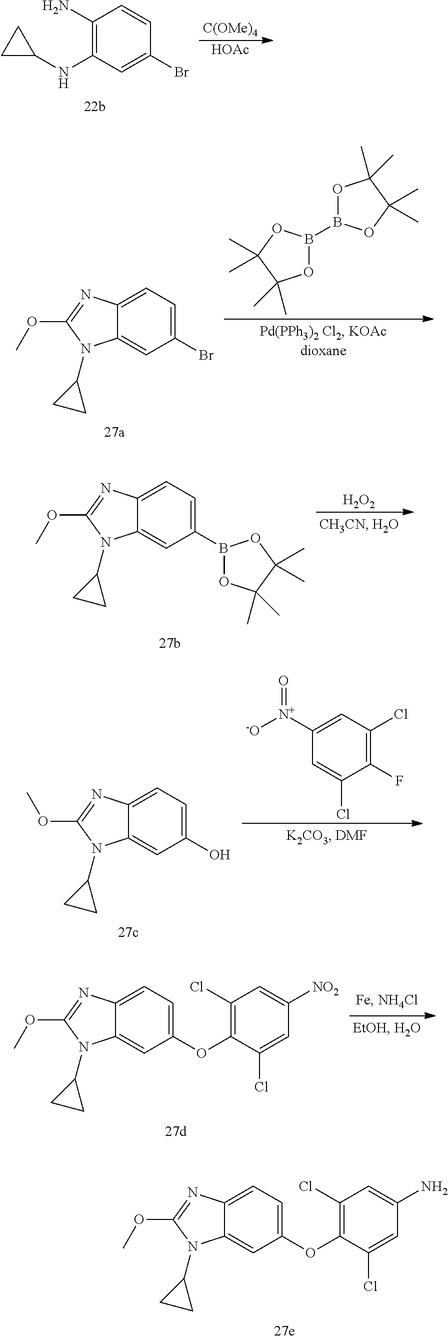

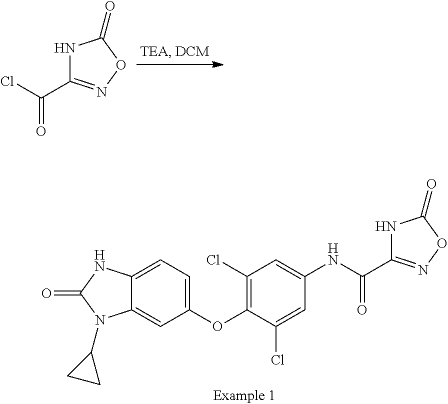 [SMILES: O=C(Cl)c1noc(=O)[nH]1 ; O=C(Nc1cc(Cl)c(Oc2ccc3[nH]c(=O)n(C4CC4)c3c2)c(Cl)c1)c1noc(=O)[nH]1]

Synthesis of 6-bromo-1-cyclopropyl-2-methoxy-1H-benzo[d]imidazole (27a). To a solution of 5-bromo-N1-cyclopropylbenzene-1,2-diamine (22b) (1 g, 4.40 mmol) in AcOH (10 mL) was added tetramethoxymethane (1.20 g, 8.81 mmol). The mixture was stirred at 50° C. for 1 hour. LCMS showed 22b was consumed completely and the desired MS was detected. The reaction mixture was concentrated under reduced pressure to remove AcOH. The residue was diluted with H₂O (15 mL) and extracted with Ethyl acetate (25 mL*2). The combined organic layers were washed with brine (15 mL*3), dried with anhydrous Na₂SO₄, filtered and concentrated under reduced pressure. The residue was purified by column chromatography (SiO₂, Petroleum ether: Ethyl acetate) to give 27a. MS mass calculated for [M+1]⁺ (C₁₁H₁₁BrN₂O) requires m/z 267.0, LCMS found m/z 267.1; ¹H NMR (400 MHz, CD₃OD) δ 7.55 (d, J=1.4 Hz, 1H), 7.22-7.34 (m, 2H), 4.16 (s, 3H), 3.08 (tt, J=7.0, 3.6 Hz, 1H), 1.09-1.20 (m, 2H), 0.94-1.02 (m, 2H).

Synthesis of 1-cyclopropyl-2-methoxy-6-(4,4,5,5-tetramethyl-1,3,2-dioxaborolan-2-yl)-1H-benzo[d]imidazole (27b). To a solution of 6-bromo-1-cyclopropyl-2-methoxy-1H-benzo[d]imidazole (27a) (100 mg, 374.36 umol) and 4,4,5,5-tetramethyl-2-(4,4,5,5-tetramethyl-1,3,2-dioxaborolan-2-yl)-1,3,2-dioxaborolane (114.08 mg, 449.23 umol) in dioxane (3 mL) was added KOAc (183.70 mg, 1.87 mmol) and Pd(PPh₃)₂Cl₂ (26.28 mg, 37.44 umol) at 20° C. under N₂. The mixture was stirred at 90° C. for 4 hours. LCMS showed 27a was consumed completely and the desired MS was detected. The suspension was filtered through a pad of Celite and the pad cake was washed with Ethyl acetate (5 mL*3). The combined filtrates were concentrated in vacuum. The residue was diluted with H₂O (10 mL) and extracted with Ethyl acetate (20 mL*2). The combined organic layers were washed with brine (15 mL*3), dried with anhydrous Na₂SO₄, filtered and concentrated under reduced pressure to give 27b. MS mass calculated for [M+1]⁺ (C₁₇H₂₃BN₂O₃) requires m/z 315.2, LCMS found m/z 315.1; ¹H NMR (400 MHz, CD₃OD) δ 7.81 (s, 1H), 7.57 (br d, J=8.0 Hz, 2H), 7.36-7.44 (m, 1H), 4.17 (s, 3H), 3.11 (td, J=7.0, 3.55 Hz, 1H), 1.32-1.41 (m, 13H).

Synthesis of 1-cyclopropyl-2-methoxy-1H-benzo[d]imidazol-6-ol (27c). To a mixture of 1-cyclopropyl-2-methoxy-6-(4,4,5,5-tetramethyl-1,3,2-dioxaborolan-2-yl)-1H-benzo[d]imidazole (27b) (110 mg, 350.11 umol) in H₂O (1.5 mL) and CH₃CN (3 mL) was added ammonium carbonate (27.68 mg, 350.11 umol, 28.83 uL) and H₂O (79.38 mg, 700.22 umol, 67.27 uL, 30% purity) under N₂. The mixture was stirred at 20° C. for 1 hour. LCMS indicated 27b was consumed completely and the desired MS was detected. The residue was poured into NaHSO₃ (30 mL) and stirred for 10 minutes. The aqueous phase was extracted with ethyl acetate (10 mL*3). The combined organic phase was washed with brine (10 mL*2), dried with anhydrous Na₂SO₄, filtered and concentrated in vacuum to give 27c. MS mass calculated for [M+1]⁺ (C₁₁H₁₂N₂O₂) requires m/z 205.1, LCMS found m/z 205.1.

Synthesis of 1-cyclopropyl-6-(2,6-dichloro-4-nitrophenoxy)-2-methoxy-1H-benzo[d]imidazole (27d). To a solution of 1-cyclopropyl-2-methoxy-1H-benzo[d]imidazol-6-ol (27c) (70 mg, 342.76 umol) and 1,3-dichloro-2-fluoro-5-nitro-benzene (79.17 mg, 377.04 umol) in DMF (3 mL) was added K₂CO₃ (71.06 mg, 514.14 umol). The mixture was degassed and purged with N₂ 3 times and stirred at 20° C. for 1 hour. LCMS and TLC showed 27c was consumed completely and the desired MS was detected. The mixture was extracted with Ethyl acetate (20 mL*2) and H₂O (5 mL). The combined organic phase was washed with brine (10 mL*3), dried with anhydrous Na₂SO₄, filtered and concentrated in vacuum. The residue was purified by Prep-TLC (Petroleum ether: Ethyl acetate) to give 27d. MS mass calculated for [M+1]⁺ (C₁₇H₁₃Cl₂N₃O₄) requires m/z 394.0, LCMS found m/z 394.1; ¹H NMR (400 MHz, CD₃OD) δ 8.45 (s, 2H), 7.32 (d, J=8.6 Hz, 1H), 6.96 (d, J=2.4 Hz, 1H), 6.65 (dd, J=8.6, 2.4 Hz, 1H), 4.15 (s, 3H), 3.01-3.10 (m, 1H), 1.04-1.14 (m, 2H), 0.90-0.97 (m, 2H).

Synthesis of 3,5-dichloro-4-((1-cyclopropyl-2-methoxy-1H-benzo[d]imidazol-6-yl)oxy)aniline (27e). To a solution of 1-cyclopropyl-6-(2,6-dichloro-4-nitrophenoxy)-2-methoxy-1H-benzo[d]imidazole (27d) (120 mg, 304.41 umol) in EtOH (3 mL) and H₂O (1 mL) was added Fe (85.01 mg, 1.52 mmol) and NH₄Cl (81.41 mg, 1.52 mmol). The mixture was stirred at 80° C. for 2 hours. LCMS showed 27d was consumed completely and the desired MS was detected. The suspension was filtered through a pad of Celite and the pad cake was washed with EtOH (5 mL*3). The combined filtrates were extracted with Ethyl acetate (15 mL*2) and H₂O (5 mL). The combined organic phase was washed with brine (10 mL*3), dried with anhydrous Na₂SO₄, filtered and concentrated in vacuum. The residue was purified by Prep-TLC (Petroleum ether: Ethyl acetate) to give 27e. MS mass calculated for [M+1]⁺ (C₁₇H₁₅Cl₂N₃O₂) requires m/z 364.1, LCMS found m/z 364.1; ¹H NMR (400 MHz, CD₃OD) δ 7.98 (s, 1H), 7.53-7.70 (m, 1H), 7.28 (d, J=8.6 Hz, 1H), 6.73-6.81 (m, 3H), 6.64 (br d, J=8.6 Hz, 1H), 4.12 (s, 3H), 2.97-3.05 (m, 3H), 2.86 (s, 2H), 1.07 (br d, J=5.8 Hz, 2H), 0.91 (br s, 2H).

Synthesis of N-(3,5-dichloro-4-((3-cyclopropyl-2-oxo-2,3-dihydro-1H-benzo[d]imidazol-5-yl)oxy)phenyl)-5-oxo-4,5-dihydro-1,2,4-oxadiazole-3-carboxamide (Example 1). To a solution of 3,5-dichloro-4-((l-cyclopropyl-2-methoxy-1H-benzo[d]imidazol-6-yl)oxy)aniline (27e) (50 mg, 137.28 umol) in DCM (2 mL) was added TEA (41.67 mg, 411.83 umol, 57.32 uL) and 5-oxo-4H-1,2,4-oxadiazole-3-carbonyl chloride (30.58 mg, 205.92 umol). The mixture was stirred at 25° C. for 0.5 hours. LCMS showed 27e was consumed completely and the desired MS was detected. The mixture was quenched with H₂O (1 mL) and MeOH (5 mL). The mixture was concentrated in vacuum. The residue was purified by Prep-HPLC ((NH₄HCO₃) column: Waters Xbridge BEH C18 100*30 mm*10 um; mobile phase: [water (10 mM NH₄HCO₃)—MeCN]) to give Example 1. MS mass calculated for [M+1]⁺ (C₁₇H₁₅Cl₂N₃O₂) requires m/z 462.0, LCMS found m/z 461.9; ¹H NMR (400 MHz, DMSO-d₆) δ 10.82 (br s, 1H), 10.65 (s, 1H), 8.10 (s, 2H), 6.91-7.26 (m, 3H), 6.83 (d, J=8.4 Hz, 1H), 6.76 (d, J=2.0 Hz, 1H), 6.30 (dd, J=8.4, 2.4 Hz, 1H), 2.81 (br s, 1H), 0.97 (br d, J=5.4 Hz, 2H), 0.81 (br s, 2H).

### Example 2

### 2-(3,5-dichloro-4-((1-cyclopropyl-2-methoxy-1H-benzo[d]imidazol-6-yl)oxy)phenyl)-3,5-dioxo-2,3,4,5-tetrahydro-1,2,4-triazine-6-carbonitrile

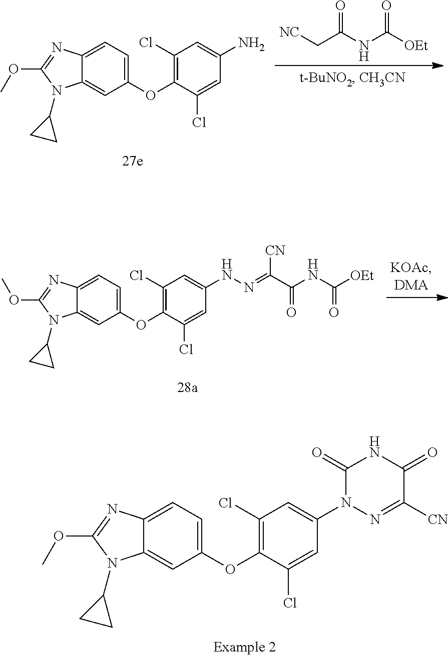 [SMILES: COc1nc2ccc(Oc3c(Cl)cc(N)cc3Cl)cc2n1C1CC1 ; CCOC(=O)NC(=O)CC#N ; CCOC(=O)NC(=O)/C(C#N)=N/Nc1cc(Cl)c(Oc2ccc3nc(OC)n(C4CC4)c3c2)c(Cl)c1 ; COc1nc2ccc(Oc3c(Cl)cc(-n4nc(C#N)c(=O)[nH]c4=O)cc3Cl)cc2n1C1CC1]

Synthesis of (E)-ethyl (2-cyano-2-(2-(3,5-dichloro-4-((1-cyclopropyl-2-methoxy-1H-benzo[d]imidazol-6-yl)oxy)phenyl)hydrazono)acetyl)carbamate (28a). To a mixture of 3,5-dichloro-4-((1-cyclopropyl-2-methoxy-1H-benzo[d]imidazol-6-yl)oxy)aniline (27e) (180 mg, 494.20 umol) and ethyl (2-cyanoacetyl)carbamate (84.88 mg, 543.62 umol) in CH₃CN (6 mL) was added t-BuONO (101.92 mg, 988.40 umol, 117.56 uL) at 0° C. Then the mixture was stirred at 0° C. for 1 hour. LCMS showed 27e was consumed completely and the desired MS was detected. The mixture was concentrated in vacuum to give 28a. MS mass calculated for [M+1]⁺ (C₂₃H₂₀Cl₂N₆O₅) requires m/z 531.1, LCMS found m/z 531.1.

Synthesis of 2-(3,5-dichloro-4-((1-cyclopropyl-2-methoxy-1H-benzo[d]imidazol-6-yl)oxy)phenyl)-3,5-dioxo-2,3,4,5-tetrahydro-1,2,4-triazine-6-carbonitrile (Example 2). To a solution of (E)-ethyl (2-cyano-2-(2-(3,5-dichloro-4-((1-cyclopropyl-2-methoxy-1H-benzo[d]imidazol-6-yl)oxy)phenyl)hydrazono)acetyl)carbamate (28a) (260 mg, 489.32 umol) in DMA (3 mL) was added KOAc (96.04 mg, 978.64 umol). The mixture was stirred at 115° C. for 3 hours. LCMS showed 28a were consumed completely and the desired MS was detected. The suspension was filtered through a pad of Celite and the pad cake was washed with MeOH (5 mL*3). The combined filtrates were concentrated in vacuum. The residue was purified by Prep-HPLC ((FA) column: Welch Ultimate C18 150*25 mm*5 um; mobile phase: [water (0.2%FA)-ACN]) to give Example 2. MS mass calculated for [M+1]⁺ (C₂₁H₁₄Cl₂N₆O₄) requires m/z 485.0, LCMS found m/z 484.9; ¹H NMR (400 MHz, DMSO-d₆) δ 7.82 (s, 2H), 7.31 (d, J=8.6 Hz, 1H), 6.90 (d, J=2.6 Hz, 1H), 6.55 (dd, J=8.6, 2.6 Hz, 1H), 4.07 (s, 3H), 3.10 (tt, J=7.0, 3.6 Hz, 1H), 1.01-1.07 (m, 2H), 0.85-0.90 (m, 2H).

### Example 3

### N-(3,5-dichloro-4-((2-methoxy-1-methyl-1H-benzo[d]imidazol-6-yl)oxy)phenyl)-5-oxo-4,5-dihydro-1,2,4-oxadiazole-3-carboxamide

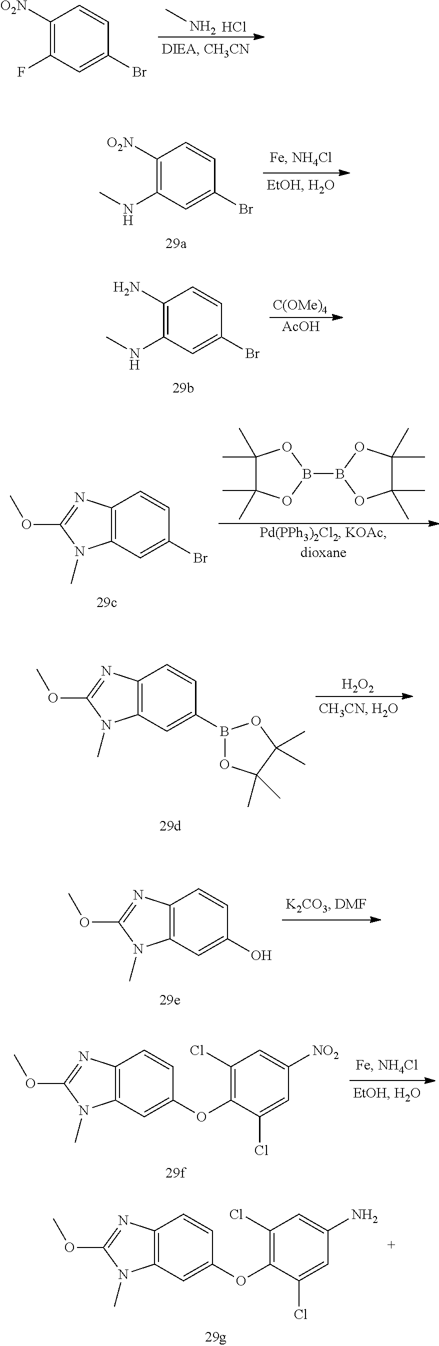

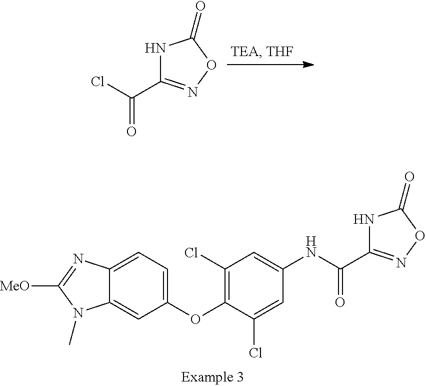 [SMILES: O=C(Cl)c1noc(=O)[nH]1 ; COc1nc2ccc(Oc3c(Cl)cc(NC(=O)c4noc(=O)[nH]4)cc3Cl)cc2n1C]

Synthesis of 5-bromo-N-methyl-2-nitroaniline (29a). To a solution of 4-bromo-2-fluoro-1-nitrobenzene (1 g, 4.55 mmol) in CH₃CN (25 mL) was added DIEA (2.94 g, 22.7 mmol, 3.96 mL) and methanamine (1.23 g, 18.3 mmol, HCl). The mixture was stirred at 50° C. for 1 hour. TLC indicated the starting material was consumed completely and one new spot was formed. The reaction mixture was partitioned between Ethyl acetate (25 mL) and H₂O (25 mL). The organic phase was separated, washed with sat. NaCl (25 mL), dried over Na₂SO₄, filtered and concentrated under reduced pressure to give 29a. ¹H NMR (400 MHz, CD₃Cl) δ 11.25-11.44 (m, 1H), 8.04 (br d, J=9.0 Hz, 1H), 8.01-8.08 (m, 1H), 7.02 (s, 1H), 6.78 (br d, J=8.6 Hz, 1H), 3.63-3.71 (m, 1H), 3.07-3.13 (m, 1H), 3.01-3.05 (m, 3H), 1.46 (d, J=6.6 Hz, 1 H).

Synthesis of 5-bromo-N1-methylbenzene-1,2-diamine (29b). To a solution of 5-bromo-N-methyl-2-nitroaniline (29a) (1.05 g, 4.54 mmol) in EtOH (30 mL) and H₂O (10 mL) was added NH₄Cl (1.22 g, 22.7 mmol) and Fe (1.27 g, 22.7 mmol). The mixture was stirred at 80° C. for 2 hours. TLC indicated 29a was consumed completely and one new spot was formed. The reaction mixture was filtered and then the filtrate was extracted with Ethyl acetate (10 mL*3). The combined organic layers were washed with sat. NaCl (5 mL), dried over Na₂SO₄, filtered and concentrated under reduced pressure to give 29b. ¹H NMR (400 MHz, CDCl₃) δ 6.66-6.72 (m, 1H), 6.63-6.66 (m, 1H), 6.46-6.51 (m, 1H), 2.99-3.35 (m, 2H), 2.71-2.82 (m, 3H).

Synthesis of 6-bromo-2-methoxy-1-methyl-1H-benzo[d]imidazole (29c). To a solution of 5-bromo-N1-methylbenzene-1,2-diamine (29b) (400 mg, 1.99 mmol) in AcOH (6 mL) was added tetramethoxymethane (2.17 g, 15.9 mmol). The mixture was stirred at 50° C. for 1 hour. LCMS showed 29b was consumed completely and one main peak with the desired MS was detected. The reaction mixture was quenched by addition NaHCO₃ (30 mL) at 0° C., and then extracted with Ethyl acetate (30 mL*3). The combined organic layers were washed with sat. NaCl (30 mL), dried over Na₂SO₄, filtered and concentrated under reduced pressure. The residue was purified by column chromatography (SiO₂, Petroleum ether: Ethyl acetate) to give 29c. ¹H NMR (400 MHz, CDCl₃) δ 7.29-7.37 (m, 1H), 7.15-7.24 (m, 2H), 4.10-4.15 (m, 3H), 3.43-3.49 (m, 3 H).

Synthesis of 2-methoxy-1-methyl-6-(4,4,5,5-tetramethyl-1,3,2-dioxaborolan-2-yl)-1H-benzo[d]imidazole (29d). To a solution of 6-bromo-2-methoxy-1-methyl-1H-benzo[d]imidazole (29c) (330 mg, 1.37 mmol) and 4,4,4′,4′,5,5,5′,5′-octamethyl-2,2′-bi(1,3,2-dioxaborolane) (1.04 g, 4.11 mmol) in dioxane (10 mL) was added Pd(PPh₃)₂Cl₂ (96.1 mg, 136.9 umol) and KOAc (1.34 g, 13.7 mmol). The mixture was stirred at 120° C. for 16 hours. LCMS showed 29c was consumed completely and one main peak with desired MS was detected. The reaction mixture was filtered and the filtrate was extracted with Ethyl acetate (30 mL*3). The combined organic layers were washed with sat. NaCl (30 mL), dried over Na₂SO₄, filtered and concentrated under reduced pressure to give 29d. MS mass calculated for [M+1]⁺ (C₁₅H₂₁BN₂O₃) requires m/z 289.2, LCMS found m/z 289.2; ¹H NMR (400 MHz, CDCl₃) δ 7.54-7.61 (m, 2H), 7.45-7.49 (m, 1H), 4.12-4.16 (m, 3H), 3.48-3.52 (m, 3H), 1.17-1.23 (m, 12H).

Synthesis of 2-methoxy-1-methyl-1H-benzo[d]imidazol-6-ol (29e). To a solution of 2-methoxy-1-methyl-6-(4,4,5,5-tetramethyl-1,3,2-dioxaborolan-2-yl)-1H-benzo[d]imidazole (29d) (240 mg, 1.17 mmol) in CH₃CN (5 mL) was added a solution of NH₄HCO₃ (92.11 mg, 1.17 mmol, 95.9 uL) in H₂O (2 mL) and H₂O₂ (264 mg, 2.33 mmol, 224 uL, 30% purity). The mixture was stirred at 20° C. for 2 hours. LCMS showed 29d was consumed completely and one main peak with desired MS was detected. The reaction mixture was quenched by addition Na₂S₂O₃ (10 mL), and then extracted with Ethyl acetate (10 mL*3). The combined organic layers were washed with sat. NaCl (10 mL), dried over Na₂SO₄, filtered and concentrated under reduced pressure to give 29e. MS mass calculated for [M+1]⁺ (C₉H₁₀N₂O₂) requires m/z 179.1, LCMS found m/z 179.1; ¹H NMR (400 MHz, CDCl₃) 67.24-7.33 (m, 1H), 6.55-6.66 (m, 2H), 4.08-4.11 (m, 3H), 3.38-3.49 (m, 3H), 1.94-2.05 (m, 3H), 1.76-1.91 (m, 3H).

Synthesis of 6-(2,6-dichloro-4-nitrophenoxy)-2-methoxy-1-methyl-1H-benzo[d]imidazole (29f). To a solution of 2-methoxy-1-methyl-1H-benzo[d]imidazol-6-ol (29e) (240 mg, 1.35 mmol) in DMF (5 mL) was added K₂CO₃ (279 mg, 2.02 mmol) and 1,3-dichloro-2-fluoro-5-nitrobenzene (311 mg, 1.48 mmol). The mixture was stirred at 20° C. for 1 hour. LCMS showed 29e was consumed completely and one main peak with the desired MS was detected. The reaction mixture was quenched by addition of H₂O (5 mL) and extracted with Ethyl acetate (10 mL*3). The combined organic layers were washed with sat. NaCl (5 mL), dried over Na₂SO₄, filtered and concentrated under reduced pressure. The residue was purified by prep-TLC (SiO₂, Petroleum ether: Ethyl acetate) to give 29f. MS mass calculated for [M+1]⁺ (C₁₅H₁₁Cl₂N₃O₄) requires m/z 368.0, LCMS found m/z 368.0; ¹H NMR (400 MHz, CDCl₃) δ 8.20-8.44 (m, 2H), 7.40-7.53 (m, 1H), 6.55-6.74 (m, 2H), 4.13-4.29 (m, 3H), 3.45-3.63 (m, 3H).

Synthesis of 3,5-dichloro-4-((2-methoxy-1-methyl-1H-benzo[d]imidazol-6-yl)oxy)aniline (29g). To a solution of 6-(2,6-dichloro-4-nitrophenoxy)-2-methoxy-1-methyl-1H-benzo[d]imidazole (290 (160 mg, 435 umol) in EtOH (3 mL) was added Fe (121 mg, 2.17 mmol) and NH₄Cl (116 mg, 2.17 mmol) in H₂O (1 mL). The mixture was stirred at 80° C. for 2 hours. LCMS showed 29f was consumed completely and one main peak with the desired MS was detected. The reaction mixture was filtered and then the filtrate was extracted with Ethyl acetate (10 mL*3). The combined organic layers were washed with sat. NaCl (5 mL), dried over Na₂SO₄, filtered and concentrated under reduced pressure to give 29g. ¹H NMR (400 MHz, CDCl₃) δ 7.31-7.36 (m, 1H), 6.63-6.65 (m, 2H), 6.55-6.62 (m, 2H), 4.07-4.12 (m, 3H), 3.60-3.75 (m, 2H), 3.35-3.46 (m, 3H).

Synthesis of N-(3,5-dichloro-4-((2-methoxy-1-methyl-1H-benzo[d]imidazol-6-yl)oxy)phenyl)-5-oxo-4,5-dihydro-1,2,4-oxadiazole-3-carboxamide (Example 3). To a solution of 3,5-dichloro-4((2-methoxy-1-methyl-1H-benzo[d]imidazol-6-yl)oxy)aniline (29g) (10 mg, 29.6 umol) in THF (1 mL) was added Et₃N (15.0 mg, 148 umol, 20.6 uL) and 5-oxo-4,5-dihydro-1,2,4-oxadiazole-3-carbonyl chloride (8.78 mg, 59.2 umol). The mixture was stirred at 20° C. for 20 minutes. LCMS showed 29g was consumed completely and one main peak with the desired MS was detected. The reaction mixture was concentrated under reduced pressure. The residue was purified by prep-HPLC (HCl condition: column: column: Welch Xtimate C18 150*25 mm*5um; mobile phase: [water(0.04% HCl)-ACN]) to give Example 3. MS mass calculated for [M+1]⁺ (C₁₈H₁₃Cl₂N₅O₅) requires m/z 450.0, LCMS found m/z 449.9; ¹H NMR (400 MHz, CD₃OD) δ 7.74-7.85 (m, 2H), 7.20-7.24 (m, 1H), 6.59-6.60 (m, 1H), 6.55-6.58 (m, 1H), 4.03-4.06 (m, 3H), 3.33-3.37 (m, 3H).

### Example 4

### N-(3,5-dichloro-4-((3-isopropyl-2-oxo-2,3-dihydro-1H-benzo[d]imidazol-5-yl)oxy)phenyl)-5-oxo-4,5-dihydro-1,2,4-oxadiazole-3-carboxamide

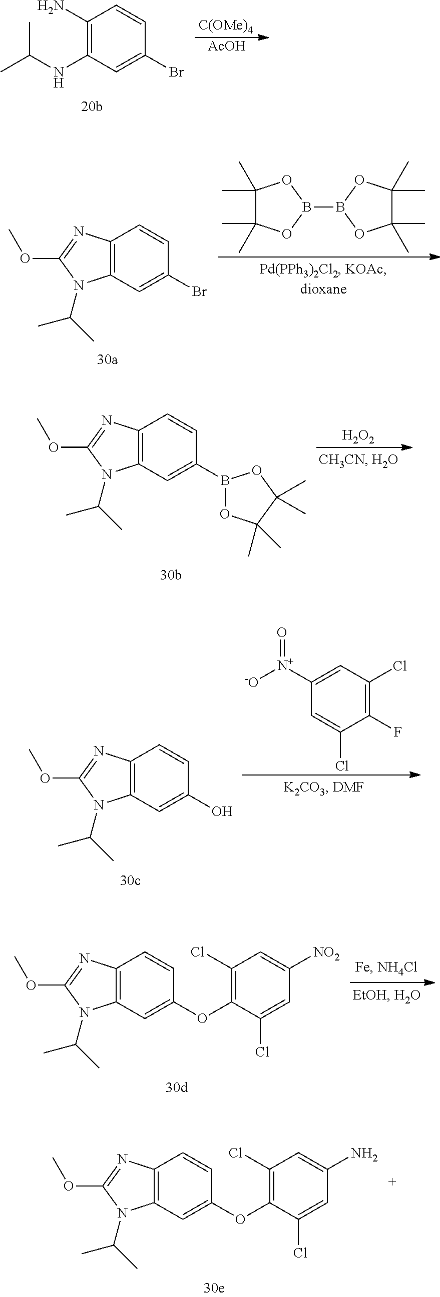

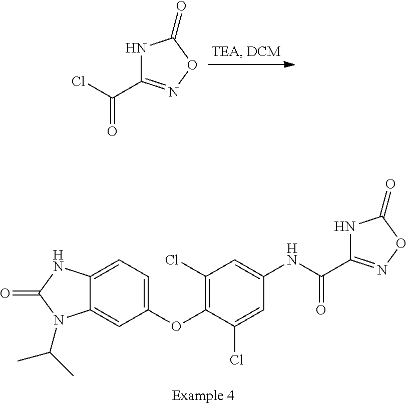 [SMILES: O=C(Cl)c1noc(=O)[nH]1 ; CC(C)n1c(=O)[nH]c2ccc(Oc3c(Cl)cc(NC(=O)c4noc(=O)[nH]4)cc3Cl)cc21]

Synthesis of 6-bromo-1-isopropyl-2-methoxy-1H-benzo[d]imidazole (30a). To a solution of 5-bromo-N1-isopropylbenzene-1,2-diamine (20b) (400 mg, 1.75 mmol) in AcOH (5 mL) was added tetramethoxymethane (1.90 g, 13.97 mmol). The mixture was stirred at 50° C. for 1 hour. LC-MS showed 20b was consumed completely and one main peak with the desired mass was detected. The reaction mixture was quenched by addition NaHCO₃ 30 mL at 0° C., and then extracted with Ethyl acetate (30 mL*3). The combined organic layers were washed with sat. NaCl (30 mL), dried over Na₂SO₄, filtered and concentrated under reduced pressure. The residue was purified by column chromatography (SiO₂, Petroleum ether: Ethyl acetate) to give 30a. MS mass calculated for [M+1]⁺ (C₁₁H₁₃BrN₂O) requires m/z 269.0, LCMS found m/z 269.0; ¹H NMR (400 MHz, CDCl₃) δ 7.31-7.53 (m, 2H), 7.26 (s, 1H), 4.52-4.71 (m, 1H), 4.20 (s, 3H), 1.50-1.60 (m, 6H).

Synthesis of 1-isopropyl-2-methoxy-6-(4,4,5,5-tetramethyl-1,3,2-dioxaborolan-2-yl)-1H-benzo[d]imidazole (30b). To a solution of 6-bromo-1-isopropyl-2-methoxy-1H-benzo[d]imidazole (30a) (320 mg, 1.19 mmol) and 4,4,4′,4′,5,5,5′,5′-octamethyl-2,2′-bi(1,3,2-dioxaborolane) (905.78 mg, 3.57 mmol) in dioxane (15 mL) was added Pd(PPh₃)₂Cl₂ (83.45 mg, 118.90 umol) and KOAc (1.17 g, 11.89 mmol). The mixture was stirred at 120° C. for 16 hours. LC-MS showed 30a was consumed completely and one main peak with the desired mass was detected. The reaction mixture was filtered and then 30 mL of H₂O was added to the filtrate. The aquoues layer was extracted with Ethyl acetate (30 mL*3). The combined organic layers were washed with sat. NaCl (30 mL), dried over Na₂SO₄, filtered and concentrated under reduced pressure to give 30b. MS mass calculated for [M+1]⁺ (C₁₇H₂₅BN₂O₃) requires m/z 317.2, LCMS found m/z 317.1.

Synthesis of 1-isopropyl-2-methoxy-1H-benzo[d]imidazol-6-ol (30c). To a solution of 1-isopropyl-2-methoxy-6-(4,4,5,5-tetramethyl-1,3,2-dioxaborolan-2-yl)-1H-benzo[d]imidazole (30b) (375 mg, 1.19 mmol) in H₂O (2 mL) was added NH₄HCO₃ (93.76 mg, 1.19 mmol, 97.67 uL) in CH₃CN (5 mL) and H₂O₂ (268.89 mg, 2.37 mmol, 227.88 uL, 30% purity). The mixture was stirred at 20° C. for 2 hours. LCMS showed 30b was consumed completely and one main peak with the desired mass was detected. The reaction mixture was quenched by addition Na₂S₂O₃ (10 mL), and then extracted with Ethyl acetate (10 mL*3). The combined organic layers were washed with sat. NaCl (10 mL), dried over Na₂SO₄, filtered and concentrated under reduced pressure to give 30c. MS mass calculated for [M+1]⁺ (C₁₁H₁₄N₂O₂) requires m/z 207.1, LCMS found m/z 207.1.

Synthesis of 6-(2,6-dichloro-4-nitrophenoxy)-1-isopropyl-2-methoxy-1H-benzo[d]imidazole (30d). To a solution of 1-isopropyl-2-methoxy-1H-benzo[d]imidazol-6-ol (30c) (244 mg, 1.18 mmol) in DMF (1 mL) was added K₂CO₃ (245.26 mg, 1.77 mmol) and 1,3-dichloro-2-fluoro-5-nitrobenzene (273.28 mg, 1.30 mmol). The mixture was stirred at 20° C. for 1 hour. LCMS showed 30c was consumed completely and one main peak with the desired mass was detected. The reaction mixture was quenched by addition of H₂O (5 mL), and then extracted with Ethyl acetate (10 mL*3). The combined organic layers were washed with sat. NaCl (5 mL), dried over Na₂SO₄, filtered and concentrated under reduced pressure. The residue was purified by prep-TLC (SiO₂, Petroleum ether: Ethyl acetateto give 30d. MS mass calculated for [M+1]⁺ (C₁₇H₁₅Cl₂N₃O₄) requires m/z 396.0, LCMS found m/z 396.1; ¹H NMR (400 MHz, CDCl₃) δ 8.28-8.38 (m, 2H), 7.39-7.44 (m, 1H), 6.86-6.89 (m, 1H), 6.52-6.56 (m, 1H), 4.51-4.60 (m, 1H), 4.16-4.19 (m, 3H), 1.50-1.55 (m, 6H).

Synthesis of 3,5-dichloro-4-((1-isopropyl-2-methoxy-1H-benzo[d]imidazol-6-yl)oxy)aniline (30e). To a solution of 6-(2,6-dichloro-4-nitrophenoxy)-1-isopropyl-2-methoxy-1H-benzo[d]imidazole (30d) (150 mg, 378.57 umol) in EtOH (3 mL) was added Fe (105.71 mg, 1.89 mmol) and NH₄Cl (101.25 mg, 1.89 mmol) in H₂O (1 mL). The mixture was stirred at 80° C. for 2 hours. TLC indicated 30d was consumed completely. The reaction mixture was filtered and the filtrate was extracted with Ethyl acetate (10 mL*3). The combined organic layers were washed with sat. NaCl (5 mL), dried over Na₂SO₄, filtered and concentrated under reduced pressure to give 30e. ¹H NMR (400 MHz, CDCl₃) δ 7.38-7.41 (m, 1H), 6.83-6.86 (m, 1H), 6.70-6.73 (m, 1H), 6.57-6.62 (m, 1H), 4.51-4.58 (m, 1H), 4.14-4.18 (m, 2H), 3.74-3.77 (m, 1H), 1.49-1.54 (m, 6H), 1.24-1.29 (m, 2H).

Synthesis of N-(3,5-dichloro-4-((3-isopropyl-2-oxo-2,3-dihydro-1H-benzo[d]imidazol-5-yl)oxy)phenyl)-5-oxo-4,5-dihydro-1,2,4-oxadiazole-3-carboxamide (Example 4). To a solution of 3,5-dichloro-4-((1-isopropyl-2-methoxy-1H-benzo[d]imidazol-6-yl)oxy)aniline (30e) (30 mg, 81.91 umol) in DCM (0.5 mL) was added TEA (24.87 mg, 245.73 umol, 34.20 uL) and 5-oxo-4H-1,2,4-oxadiazole-3-carbonyl chloride (18.25 mg, 122.87 umol). The mixture was stirred at 25° C. for 0.5 hours. LCMS showed 30e was consumed completely and trace desired MS was detected. The mixture was stirred for another 2 hours. LCMS showed the reaction was completed. The reaction mixture was quenched with MeOH (5 mL) and concentrated in vacuum. The residue was purified by Prep-HPLC ((FA) column: Welch Xtimate C18 150*25 mm*5 um; mobile phase: [water (0.2%FA)-ACN]) to give crude product. The crude product was purified by Prep-TLC (SiO₂, Petroleum ether: Ethyl acetate) to give Example 4. MS mass calculated for [M+1]⁺ (C₁₉H₁₅Cl₂N₅O₅) requires m/z 464.0, LCMS found m/z 464.0; 1 H NMR (400 MHz, CD₃OD) δ 7.96 (s, 2H), 6.95 (d, J=8.6 Hz, 1H), 6.81 (d, J=2.2 Hz, 1H), 6.44 (dd, J=8.6, 2.4 Hz, 1H), 4.61 (dq, J=14.0, 6.8 Hz, 1H), 1.48 (d, J=7.0 Hz, 6H).

### Example 5

### 2-(3,5-dichloro-4-((3-isopropyl-2-oxo-2,3-dihydro-1H-benzo[d]imidazol-5-yl)oxy)phenyl)-3,5-dioxo-2,3,4,5-tetrahydro-1,2,4-triazine-6-carbonitrile

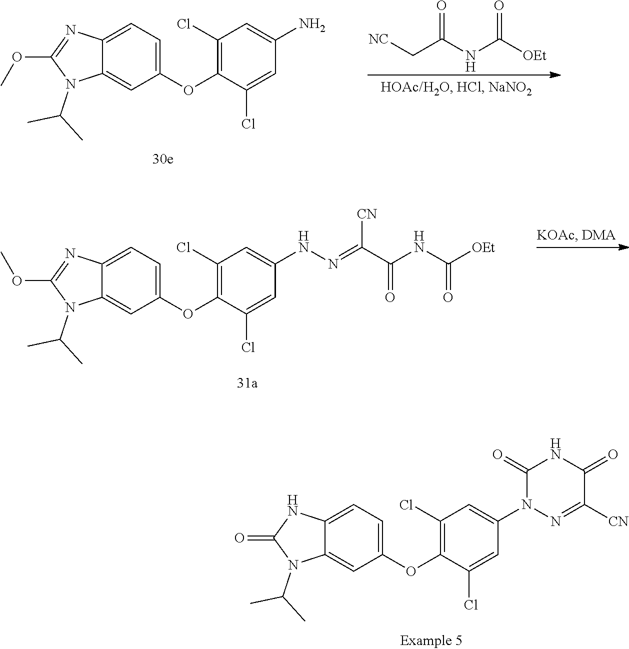 [SMILES: COc1nc2ccc(Oc3c(Cl)cc(N)cc3Cl)cc2n1C(C)C ; CCOC(=O)NC(=O)CC#N ; CCOC(=O)NC(=O)/C(C#N)=N/Nc1cc(Cl)c(Oc2ccc3nc(OC)n(C(C)C)c3c2)c(Cl)c1 ; CC(C)n1c(=O)[nH]c2ccc(Oc3c(Cl)cc(-n4nc(C#N)c(=O)[nH]c4=O)cc3Cl)cc21]

Synthesis of (E)-ethyl (2-cyano-2-(2-(3,5-dichloro-4-((1-isopropyl-2-methoxy-1H-benzo[d]imidazol-6-yl)oxy)phenyl)hydrazono)acetyl)carbamate (31a). To a solution of 3,5-dichloro-4-((1-isopropyl-2-methoxy-1H-benzo[d]imidazol-6-yl)oxy)aniline (30e) (35 mg, 95.57 umol) in HOAc (2 mL) and H₂O (1 mL) was added ethyl (2-cyanoacetyl)carbamate (16.86 mg, 107.99 umol) at 0° C. Next, HCl (1 M, 23.89 uL) was added dropwise at 2-4° C., and then the mixture was stirred at 0° C. for 10 minutes. A solution of NaNO₂ (8.57 mg, 124.24 umol) in H₂O (0.05 mL) was added to the reaction mixture dropwise at 0° C. Then the mixture was stirred at 0° C. for 6 hours. LCMS showed 30e was consumed completely and one main peak with the desired mass was detected. The reaction mixture was quenched by addition H₂O (5 mL), and then extracted with Ethyl acetate (10 mL*3). The combined organic layers were washed with brine (5 mL), dried over Na₂SO₄, filtered and concentrated under reduced pressure to give 31a. MS mass calculated for [M+1]⁺ (C₂₃H₂₂Cl₂N₆O₅) requires m/z 533.1, LCMS found m/z 533.1; ¹H NMR (400 MHz, DMSO-d₆) δ 11.03-11.06 (m, 1H), 7.35-7.38 (m, 1H), 7.02-7.03 (m, 1H), 6.56-6.60 (m, 1H), 4.23-4.26 (m, 2H), 4.18-4.20 (m, 2H), 4.15-4.17 (m, 2H), 1.45-1.51 (m, 6 H).

Synthesis of 2-(3,5-dichloro-4-((3-isopropyl-2-oxo-2,3-dihydro-1H-benzo[d]imidazol-5-yl)oxy)phenyl)-3,5-dioxo-2,3,4,5-tetrahydro-1,2,4-triazine-6-carbonitrile (Example 5). To a solution of (E)-ethyl (2-cyano-2-(2-(3,5-dichloro-4-((1-isopropyl-2-methoxy-1H-benzo[d]imidazol-6-yl)oxy)phenyl)hydrazono)acetyl)carbamate (31a) (20 mg, 37.50 umol) in DMA (1 mL) was added KOAc (7.36 mg, 75.00 umol). The mixture was stirred at 110° C. for 6 hours. LCMS showed 31a was consumed completely and one main peak with desired mass was detected. The reaction mixture was quenched by addition H₂O (5 mL), and then extracted with Ethyl acetate (10 mL*3). The combined organic layers were washed with brine (5 mL), dried over Na₂SO₄, filtered and concentrated under reduced pressure to give a residue. The residue was purified by prep-HPLC (column: Welch Xtimate C18 150*25 mm*5 um; mobile phase: [water (0.2%FA)-ACN]) to give Example 5. MS mass calculated for [M+H]⁺ (C₂₀H₁₄Cl₂N₆O₄) requires m/z 473.0, MS found m/z 473.1; ¹H NMR (400 MHz, DMSO-d₆) δ ppm 10.76 (s, 1H), 7.80 (s, 2H), 7.00 (s, 1H), 6.86 (d, J=8.4 Hz, 1H), 6.52 (s, 1H), 6.25 (dd, J=8.6, 2.4 Hz, 1H), 4.53 (dt, J=13.8, 7.0 Hz, 1H), 2.67-2.84 (m, 1H), 2.52-2.57 (m, 3H), 1.41 (d, J=7.0 Hz, 6 H).

### Example 6

### N-(3,5-dichloro-4-((1-isopropyl-2-methoxy-1H-benzo[d]imidazol-6-yl)oxy)phenyl)-5-oxo-4,5-dihydro-1,2,4-oxadiazole-3-carboxamide

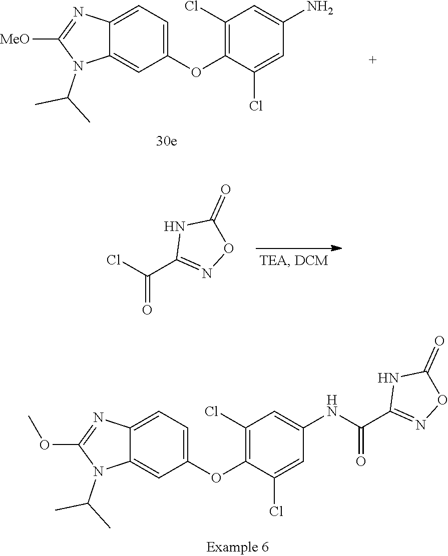 [SMILES: COc1nc2ccc(Oc3c(Cl)cc(N)cc3Cl)cc2n1C(C)C ; O=C(Cl)c1noc(=O)[nH]1 ; COc1nc2ccc(Oc3c(Cl)cc(NC(=O)c4noc(=O)[nH]4)cc3Cl)cc2n1C(C)C]

Synthesis of N-(3,5-dichloro-4-((1-isopropyl-2-methoxy-1H-benzo[d]imidazol-6-yl)oxy)phenyl)-5-oxo-4,5-dihydro-1,2,4-oxadiazole-3-carboxamide (Example 6). To a solution of 3,5-dichloro-44(1-isopropyl-2-methoxy-1H-benzo[d]imidazol-6-yl)oxy)aniline (30e) (30 mg, 81.91 umol) in DCM (0.5 mL) was added TEA (24.87 mg, 245.73 umol, 34.20 uL) and 5-oxo-4H-1,2,4-oxadiazole-3-carbonyl chloride (18.25 mg, 122.87 umol). The mixture was stirred at 25° C. for 0.5 hours. LCMS showed 30e was consumed completely and the desired MS was detected. The mixture was quenched with NaHCO₃ (10 mL) and extracted with DCM (10 mL*2). The combined organic phase was washed with brine (10 mL*3), dried with anhydrous Na₂SO₄, filtered and concentrated in vacuum. The residue was purified by Prep-HPLC ((FA) column: Phenomenex Luna C18 200*40 mm*10 um; mobile phase: [water (0.2%FA)-ACN]) to give Example 6. MS mass calculated for [M+1]⁺ (C₂₀H₁₇Cl₂N₅O₅) requires m/z 478.1, LCMS found m/z 477.9; ¹H NMR (400 MHz, DMSO-d₆) δ 10.49 (br s, 1H), 8.14 (s, 2H), 7.29 (d, J=8.6 Hz, 1H), 6.99 (d, J=1.8 Hz, 1H), 6.49 (dd, J=8.62, 1.65 Hz, 1H), 4.61 (dt, J=13.6, 6.8 Hz, 1H), 4.07 (s, 3H), 1.41 (d, J=6.8 Hz, 6H).

### Example 7

### N-(3,5-dichloro-4-((1-cyclopropyl-2-methoxy-1H-benzo[d]imidazol-6-yl)oxy)phenyl)-5-oxo-4,5-dihydro-1,2,4-oxadiazole-3-carboxamide

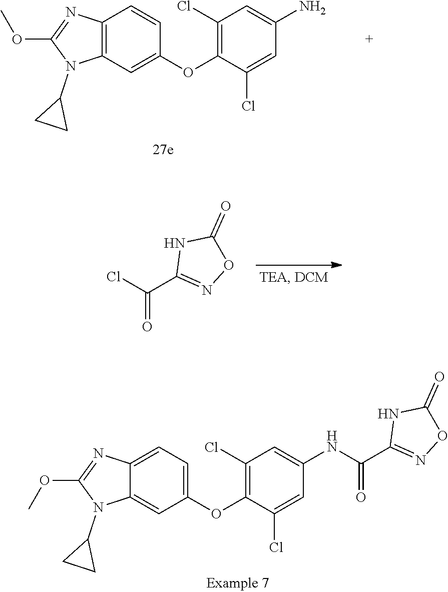 [SMILES: COc1nc2ccc(Oc3c(Cl)cc(N)cc3Cl)cc2n1C1CC1 ; O=C(Cl)c1noc(=O)[nH]1 ; COc1nc2ccc(Oc3c(Cl)cc(NC(=O)c4noc(=O)[nH]4)cc3Cl)cc2n1C1CC1]

Synthesis of N-(3,5-dichloro-4-((1-cyclopropyl-2-methoxy-1H-benzo[d]imidazol-6-yl)oxy)phenyl)-5-oxo-4,5-dihydro-1,2,4-oxadiazole-3-carboxamide (Example 7). To a solution of 3,5-dichloro-4-((l-cyclopropyl-2-methoxy-1H-benzo[d]imidazol-6-yl)oxy)aniline (27e) (20 mg, 54.91 umol) in DCM (0.5 mL) was added TEA (16.67 mg, 164.73 umol, 22.93 uL) and 5-oxo-4H-1,2,4-oxadiazole-3-carbonyl chloride (12.23 mg, 82.37 umol). The mixture was stirred at 25° C. for 0.5 hours. LCMS and HPLC showed 27e was consumed completely and the desired MS was detected. The mixture was quenched with NaHCO₃ (10 mL) and stirred for 10 minutes. The mixture was extracted with DCM (15 mL*2). The combined organic phase was washed with brine (10 mL*3), dried with anhydrous Na₂SO₄, filtered and concentrated in vacuum. The residue was purified by Prep-HPLC ((NH₄HCO₃) column: Waters Xbridge BEH C18 100*25 mm*5 um; mobile phase: [water (10 mM NH₄HCO₃)-ACN]) to give Example 7. MS mass calculated for [M+1]⁺ (C₂₀H₁₅Cl₂N₅O₅) requires m/z 476.0, LCMS found m/z 475.9; ¹H NMR (400 MHz, DMSO-d₆) δ 10.59 (br s, 1H), 8.14 (s, 2H), 7.30 (d, J=8.6 Hz, 1H), 6.84 (d, J=2.4 Hz, 1H), 6.54 (dd, J=8.6, 2.6 Hz, 1H), 4.07 (s, 3H), 3.08 (tt, J=7.0, 3.6 Hz, 1H), 1.00-1.10 (m, 2H), 0.82-0.90 (m, 2H).

### Example 8

### 2-(3,5-dichloro-4-((3-cyclopropyl-2-oxo-2,3-dihydro-1H-benzo[d]imidazol-5-yl)oxy)phenyl)-3,5-dioxo-2,3,4,5-tetrahydro-1,2,4-triazine-6-carbonitrile

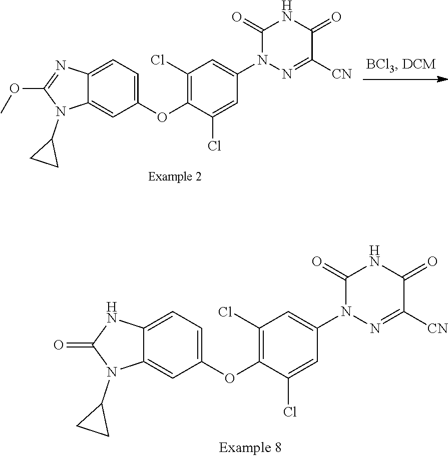 [SMILES: COc1nc2ccc(Oc3c(Cl)cc(-n4nc(C#N)c(=O)[nH]c4=O)cc3Cl)cc2n1C1CC1 ; N#Cc1nn(-c2cc(Cl)c(Oc3ccc4[nH]c(=O)n(C5CC5)c4c3)c(Cl)c2)c(=O)[nH]c1=O]

Synthesis of 2-(3,5-dichloro-4-((3-cyclopropyl-2-oxo-2,3-dihydro-1H-benzo[d]imidazol-5-yl)oxy)phenyl)-3,5-dioxo-2,3,4,5-tetrahydro-1,2,4-triazine-6-carbonitrile (Example 8). To a solution of 2-(3,5-dichloro-4-((1-cyclopropyl-2-methoxy-1H-benzo[d]imidazol-6-yl)oxy)phenyl)-3,5-dioxo-2,3,4,5-tetrahydro-1,2,4-triazine-6-carbonitrile (Example 2) (10 mg, 20.61 umol) in DCM (3 mL) was added BCl₃ (1 M, 41.21 uL). The mixture was stirred at 40° C. for 32 hours. LCMS showed Example 2 was consumed completely and the desired MS was found. The mixture was quenched with MeOH (2 mL) and stirred at 25° C. for 10 minutes. The mixture was concentrated in vacuum. The residue was purified by Prep-HPLC ((FA) column: Phenomenex Luna C18 200*40 mm*10 um; mobile phase: [water (0.2%FA)-ACN]) to give Example 8. MS mass calculated for [M+1]⁺ (C₂₀H₁₂Cl₂N₆O₄) requires m/z 471.0, LCMS found m/z 470.9; ¹H NMR (400 MHz, DMSO-d₆) δ 10.69 (s, 1H), 7.81 (s, 2H), 6.82-6.87 (m, 2H), 6.31 (dd, J=8.4, 2.4 Hz, 1H), 2.83 (tt, J=7.0, 3.6 Hz, 1H), 0.95-1.01 (m, 2H), 0.80-0.86 (m, 2H).

### Example 9

### N-(4-((1-(tert-butyl)-2-methoxy-1H-benzo[d]imidazol-6-yl)oxy)-3,5-dichlorophenyl)-5-oxo-4,5-dihydro-1,2,4-oxadiazole-3-carboxamide

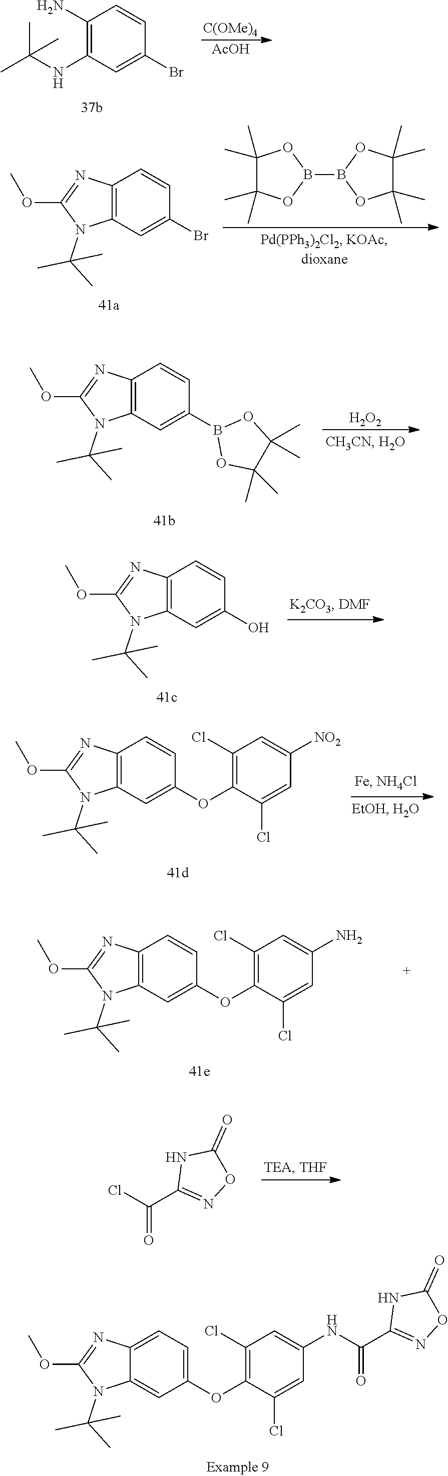

Synthesis of 6-bromo-1-(tert-butyl)-2-methoxy-1H-benzo[d]imidazole (41a). To a solution of 5-bromo-N1-(tert-butyl) benzene-1,2-diamine (37b) (1 g, 4.11 mmol) in HOAc (5 mL) was added C(OCH₃)₄ (2.24 g, 16.45 mmol). The mixture was stirred at 50° C. for 16 hours. TLC and LCMS showed the reaction was completed. The reaction mixture was quenched by addition the aqueous NaHCO₃ (50 mL) at 20° C., and then extracted with Ethyl acetate (10 mL*2). The combined organic layers were washed with brine (15 mL*2), dried over Na₂SO₄, filtered and concentrated under reduced pressure. The residue was purified by column chromatography (SiO₂, Petroleum ether: Ethyl acetate) to give 41a. MS mass calculated for [M+1]⁺ (C₁₂H₁₅BrN₂O) requires m/z 283.0, LCMS found m/z 283.0.

Synthesis of 1-(tert-butyl)-2-methoxy-6-(4,4,5,5-tetramethyl-1,3,2-dioxaborolan-2-yl)-1H-benzo[d]imidazole (41b). A mixture of 6-bromo-1-(tert-butyl)-2-methoxy-1H-benzo[d]imidazole (41a) (700 mg, 2.47 mmol), KOAc (1.21 g, 12.36 mmol), BPD (1.88 g, 7.42 mmol) and Pd(PPh₃)₂Cl₂(173.51 mg, 247.21 umol) in dioxane (3 mL) was degassed and purged with N₂ 3 times, and then the mixture was stirred at 110° C. for 16 hours under N₂ atmosphere. TLC and LCMS showed the reaction was completed. The reaction mixture was concentrated under reduced pressure to remove dioxane. The residue was diluted with water (40 mL) and extracted with Ethyl acetate (20 mL*3). The combined organic layers were washed with brine (15 mL*2), dried over Na₂SO₄, filtered and concentrated under reduced pressure. The residue was purified by column chromatography (SiO₂, Petroleum ether: Ethyl acetate) to give 41b. MS mass calculated for [M+1]⁺ (C₁₈H₂₇BN₂O₃) requires m/z 331.2, LCMS found m/z 331.2; ¹H NMR (400 MHz, CDCl₃) δ 7.99 (s, 1H), 7.59-7.64 (m, 1H), 7.51-7.55 (m, 1H), 4.19 (s, 3H), 1.79-1.83 (m, 9H), 1.36 (s, 12H).

Synthesis of 1-(tert-butyl)-2-methoxy-1H-benzo[d]imidazol-6-ol (41c). To a solution of 1-(tert-butyl)-2-methoxy-6-(4,4,5,5-tetramethyl-1,3,2-dioxaborolan-2-yl)-1H-benzo[d]imidazole (41b) (440 mg, 1.33 mmol) in ACN (10 mL) was added a solution of NH₄HCO₃ (105.34 mg, 1.33 mmol, 109.73 uL) in H₂O (5 mL) at 20° C. Then H₂O₂ (302.10 mg, 2.66 mmol, 256.02 uL, 30% purity) was added dropwise at 20° C. The resulting mixture was stirred at 20° C. for 1 hour. TLC indicated 41b was consumed completely and one new spot was formed. The mixture was poured into saturated solution of NaHSO₃ (10 mL) and stirred for 10 minutes. The aqueous phase was extracted with ethyl acetate (15 mL*3). The combined organic phase was washed with brine (10 mL), dried with anhydrous Na₂SO₄, filtered and concentrated in vacuum to give 41c. MS mass calculated for [M+1]⁺ (C₁₂H₁₆N₂O₂) requires m/z 221.1, LCMS found m/z 221.1.

Synthesis of 1-(tert-butyl)-6-(2,6-dichloro-4-nitrophenoxy)-2-methoxy-1H-benzo[d]imidazole (41d). To a solution of 1-(tert-butyl)-2-methoxy-1H-benzo[d]imidazol-6-ol (41c) (340 mg, 1.54 mmol) and 1,3-dichloro-2-fluoro-5-nitro-benzene (356.55 mg, 1.70 mmol) in DMF (20 mL) was added K₂CO₃ (320.01 mg, 2.32 mmol). The mixture was stirred at 20° C. for 1 hour. TLC indicated 41c was consumed completely and one new spot was formed. The reaction mixture was diluted with water (20 mL) and extracted with Ethyl acetate (20 mL*2). The combined organic layers were washed with brine (20 mL), dried over Na₂SO₄, filtered and concentrated under reduced pressure. The residue was purified by column chromatography (SiO₂, Petroleum ether: Ethyl acetate) to give 41d. MS mass calculated for [M+1]⁺ (C₁₈H₁₇Cl₂N₃O₄) requires m/z 410.0, LCMS found m/z 410.0; ¹H NMR (400 MHz, CDCl₃) δ 8.32 (s, 2H), 7.38 (d, J=8.8 Hz, 1H), 7.24 (d, J=2.4 Hz, 1H), 6.48 (dd, J=2.4, 8.6 Hz, 1H), 4.16 (s, 3H), 1.75 (s, 9H).

Synthesis of 4-((1-(tert-butyl)-2-methoxy-1H-benzo[d]imidazol-6-yl)oxy)-3,5-dichloroaniline (41e). To a solution of 1-(tert-butyl)-6-(2,6-dichloro-4-nitrophenoxy)-2-methoxy-1H-benzo[d]imidazole (41d) (200 mg, 487.51 umol) in EtOH (10 mL) was added Fe (136.12 mg, 2.44 mmol) and then a solution of NH₄Cl (130.39 mg, 2.44 mmol) in H₂O (4 mL) was added in the mixture dropwise. The mixture was stirred at 80° C. for 1 hour. TLC and LCMS indicated 41d was consumed completely and one new spot was formed. The reaction mixture was concentrated under reduced pressure to remove EtOH. The residue was diluted with water (5 mL) and extracted with Ethyl acetate (15 mL*2). The combined organic layers were washed with brine (10 mL*2), dried over Na₂SO₄, filtered and concentrated under reduced pressure. The residue was purified by prep-TLC (SiO₂, Petroleum ether: Ethyl acetate) to give 41e. MS mass calculated for [M+1]⁺ (C₁₈H₁₉Cl₂N₃O₂) requires m/z 380.1, LCMS found m/z 380.0.

Synthesis of N-(4-((1-(tert-butyl)-2-methoxy-1H-benzo[d]imidazol-6-yl)oxy)-3,5-dichlorophenyl)-5-oxo-4,5-dihydro-1,2,4-oxadiazole-3-carboxamide (Example 9). A mixture of 4-((1-(tert-butyl)-2-methoxy-1H-benzo[d]imidazol-6-yl)oxy)-3,5-dichloroaniline (41e) (30 mg, 78.89 umol), 5-oxo-4H-1,2,4-oxadiazole-3-carbonyl chloride (58.58 mg, 394.46 umol) and TEA (39.92 mg, 394.46 umol, 54.90 uL) in THF (3 mL) was degassed and purged with N₂ 3 times. Then the mixture was stirred at 20° C. for 1 hour under N₂ atmosphere. TLC and LCMS showed the reaction was completed. The reaction mixture was diluted with water (10 mL) and extracted with Ethyl acetate (10 mL*3). The combined organic layers were washed with brine (10 mL*2), dried over Na₂SO₄, filtered and concentrated under reduced pressure. The residue was purified by prep-HPLC (column: Waters Xbridge BEH C18 100*30 mm*10 um; mobile phase: [water (1 0mM NH₄HCO₃)-ACN]) to give Example 9. MS mass calculated for [M+1]⁺ (C₂₁H₁₉Cl₂N₅O₅) requires m/z 492.10, LCMS found m/z 492.1; ¹H NMR (400 MHz, DMSO-d₆) δ 10.87-10.95 (m, 1H), 8.06-8.12 (m, 2H), 7.25-7.31 (m, 1H), 7.13 (d, J=2.4 Hz, 1H), 6.47 (dd, J=2.3, 8.7 Hz, 1H), 4.05 (s, 3H), 1.66 (s, 9H).

### Example 10

### N-(4-((3-(tert-butyl)-2-oxo-2,3-dihydro-1H-benzo[d]imidazol-5-yl)oxy)-3,5-dichlorophenyl)-5-oxo-4,5-dihydro-1,2,4-oxadiazole-3-carboxamide

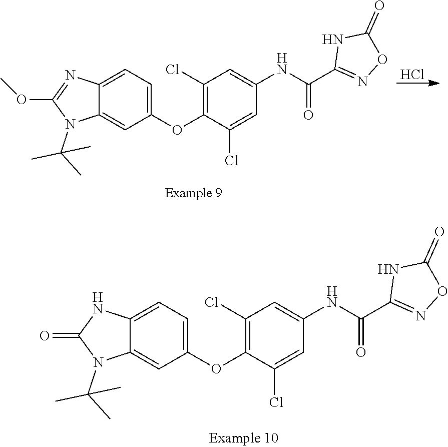 [SMILES: COc1nc2ccc(Oc3c(Cl)cc(NC(=O)c4noc(=O)[nH]4)cc3Cl)cc2n1C(C)(C)C ; Cl ; CC(C)(C)n1c(=O)[nH]c2ccc(Oc3c(Cl)cc(NC(=O)c4noc(=O)[nH]4)cc3Cl)cc21]

Synthesis of N-(4-((3-(tert-butyl)-2-oxo-2,3-dihydro-1H-benzo[d]imidazol-5-yl)oxy)-3,5-dichlorophenyl)-5-oxo-4,5-dihydro-1,2,4-oxadiazole-3-carboxamide (Example 10). To a solution of N-(4-((1-(tert-butyl)-2-methoxy-1H-benzo[d]imidazol-6-yl)oxy)-3,5-dichlorophenyl)-5-oxo-4,5-dihydro-1,2,4-oxadiazole-3-carboxamide (Example 9) (15 mg, 30.47 umol) in MeOH (2 mL) was added conc. HCl (0.2 mL). The mixture was stirred at 20° C. for 16 hours. LCMS showed the reaction was completed. The reaction mixture was concentrated under reduced pressure to remove MeOH. The residue was diluted with water (10 mL) and extracted with Ethyl acetate (10 mL*3). The combined organic layers were washed with brine (20 mL), dried over Na₂SO₄, filtered and concentrated under reduced pressure. The residue was purified by prep-HPLC (column: Waters Xbridge BEH C18 100*25 mm*5 um; mobile phase: [water (10 mM NH₄HCO₃)-ACN]) to give Example 10. MS mass calculated for [M+1]⁺ (C₂₀H₁₇Cl₂N₅O₅) requires m/z 478.1, LCMS found m/z 478.1; ¹H NMR (400 MHz, DMSO-d₆) δ 12.03-12.06 (m, 1H), 8.25-8.36 (m, 3H), 8.09-8.14 (m, 1H), 8.02-8.07 (m, 3H), 7.94-7.99 (m, 2H), 7.85 (m, 1H).

### Example 11

### N-(3,5-dichloro-4-((2-methoxy-1-(1-methylcyclopropyl)-1H-benzo[d]imidazol-6-yl)oxy)phenyl)-5-oxo-4,5-dihydro-1,2,4-oxadiazole-3-carboxamide

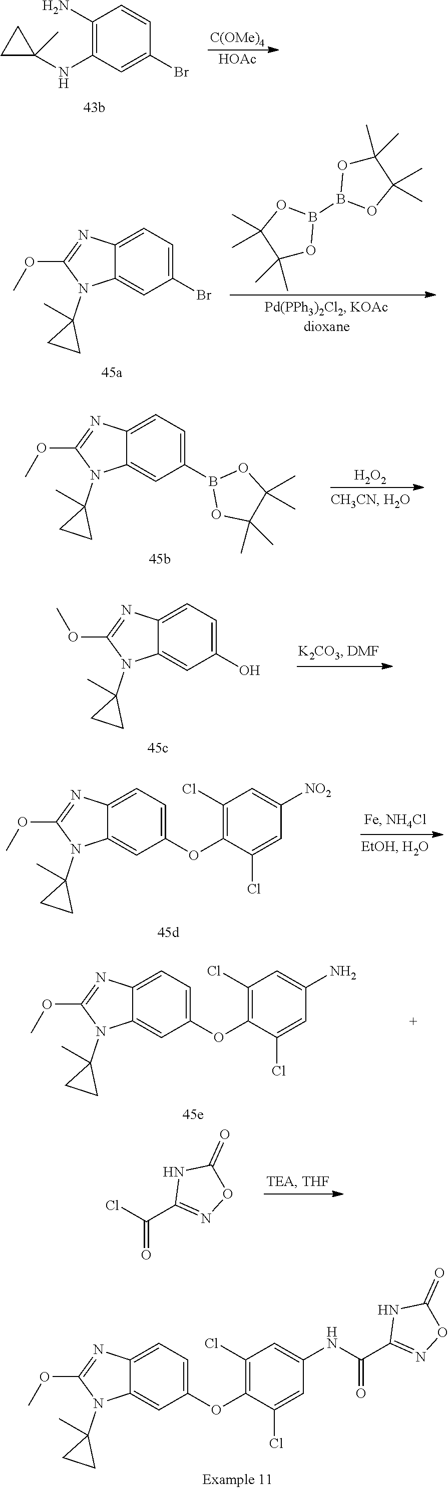

Synthesis of 6-bromo-2-methoxy-1-(1-methylcyclopropyl)-1H-benzo[d]imidazole (45a). To a solution of 5-bromo-N1-(1-methylcyclopropyl)benzene-1,2-diamine (43b) (200 mg, 829.44 umol) in AcOH (5 mL) was added tetramethoxymethane (225.85 mg, 1.66 mmol). The mixture was stirred at 50° C. for 1 hour. LCMS and TLC showed 43b was consumed completely and one new spot was formed. The reaction mixture was concentrated under reduced pressure to remove AcOH. The residue was diluted with H₂O (10 mL) and extracted with Ethyl acetate (30 mL*2). The combined organic layers were washed with brine (15 mL*3), dried with anhydrous Na₂SO₄, filtered and concentrated under reduced pressure to give a residue. The residue was purified by prep-TLC (Petroleum ether: Ethyl acetate) to give 45a. MS mass calculated for [M+1]⁺ (C₁₂H₁₃BrN₂O) requires m/z 281.0, LCMS found m/z 281.0; ¹HNMR (400 MHz, CD₃OD) δ 1.01-1.19 (m, 4H), 1.45-1.50 (m, 3H), 4.15-4.19 (m, 3H), 7.24-7.33 (m, 2H), 7.53-7.58 (m, 1H).

Synthesis of 2-methoxy-1-(1-methylcyclopropyl)-6-(4,4,5,5-tetramethyl-1,3,2-dioxaborolan-2-yl)-1H-benzo[d]imidazole (45b). To a solution of 6-bromo-2-methoxy-1-(1-methylcyclopropyl)-1H-benzo[d]imidazole (45a) (210 mg, 746.94 umol) and BPD (569.03 mg, 2.24 mmol) in dioxane (5 mL) was added Pd (PPh₃)₂Cl₂ (52.43 mg, 74.69 umol) and KOAc (733.06 mg, 7.47 mmol) at 20° C. under N₂. The mixture was stirred at 90° C. for 4 hours. TLC and LCMS showed 45a was consumed completely and the desired MS was detected. The suspension was filtered through a pad of Celite and the pad cake was washed with Ethyl acetate (10 mL*3). The combined filtrates were concentrated in vacuum. The residue was diluted with H₂O (10 mL) and extracted with Ethyl acetate (30 mL*2). The combined organic layers were washed with brine (15 mL*3), dried with anhydrous Na₂SO₄, filtered and concentrated under reduced pressure. The residue was purified by prep-TLC (Petroleum ether: Ethyl acetate) to give 45b. MS mass calculated for [M+1]⁺ (C₁₈H₂₅BN₂O₃) requires m/z 329.2, LCMS found m/z 329.1; ¹HNMR (400 MHz, CD₃OD) δ 1.05-1.11 (m, 2H), 1.17-1.22 (m, 13H), 1.23-1.26 (m, 2H), 1.35-1.40 (m, 14H), 1.48-1.52 (m, 4H), 4.19 (s, 3H), 4.76-4.94 (m, 1H), 7.39-7.43 (m, 1H), 7.42 (s, 1H), 7.55-7.59 (m, 1H), 7.80-7.82 (m, 1H).

Synthesis of 2-methoxy-1-(1-methylcyclopropyl)-1H-benzo[d]imidazol-6-ol (45c). To a mixture of 2-methoxy-1-(1-methylcyclopropyl)-6-(4,4,5,5-tetramethyl-1,3,2-dioxaborolan-2-yl)-1H-benzo[d]imidazole (45b) (205 mg, 624.59 umol) in H₂O (1.5 mL) and CH₃CN (3 mL) was added ammonium carbonate (49.38 mg, 624.59 umol, 51.43 uL) and H₂O₂ (141.62 mg, 1.25 mmol, 120.01 uL, 30% purity) under N₂. The mixture was stirred at 20° C. for 1 hour. LCMS indicated 45b was consumed completely and the desired MS was detected. The residue was poured into NaHSO₃ solution (30 mL) and stirred for 10 minutes. The aqueous phase was extracted with ethyl acetate (15 mL*3). The combined organic phase was washed with brine (10 mL*2), dried with anhydrous Na₂SO₄, filtered and concentrated in vacuum to give 45c. The crude product was used in the next step without further purification. MS mass calculated for [M+1]⁺ (C₁₂H₁₄N₂O₂) requires m/z 219.1, LCMS found m/z 219.0; ¹HNMR (400 MHz, CD₃OD) δ 7.20 (d, J=8.4 Hz, 1H), 6.83 (d, J=2.4 Hz, 1H), 6.63 (dd, J=8.6, 2.32 Hz, 1H), 4.12 (s, 3H), 1.46 (s, 3H), 1.11-1.17 (m, 3H), 0.98-1.04 (m, 2H).

Synthesis of 6-(2,6-dichloro-4-nitrophenoxy)-2-methoxy-1-(1-methylcyclopropyl)-1H-benzo[d]imidazole (45d). To a solution of 2-methoxy-1-(1-methylcyclopropyl)-1H-benzo[d]imidazol-6-ol (45c) (170 mg, 778.92 umol) and 1,3-dichloro-2-fluoro-5-nitro-benzene (179.92 mg, 856.81 umol) in DMF (3 mL) was added K₂CO₃ (161.48 mg, 1.17 mmol) at 20° C. under N₂. The mixture was stirred at 20° C. for 1 hour. TLC and LCMS showed 45c was consumed completely and the desired MS was detected. The mixture was extracted with Ethyl acetate (30 mL*2) and H₂O (10 mL). The combined organic phase was washed with brine (10 mL*3), dried with anhydrous Na₂SO₄, filtered and concentrated in vacuum to give a residue. The residue was purified by prep-TLC (Petroleum ether: Ethyl acetate) to give 45d. MS mass calculated for [M+1]⁺ (C₁₈H₁₅Cl₂N₃O₄) requires m/z 408.0, LCMS found m/z 408.0; ¹HNMR (400 MHz, CD₃OD) δ 8.44-8.46 (m, 1H), 7.33 (d, J=8.6 Hz, 1H), 6.99 (d, J=2.4 Hz, 1H), 6.62 (dd, J=8.6, 2.6 Hz, 1H), 4.15-4.18 (m, 2H), 2.98-3.00 (m, 1H), 2.85-2.87 (m, 1H), 1.44-1.47 (m, 2H), 1.19-1.21 (m, 3H).

Synthesis of 3,5-dichloro-4-((2-methoxy-1-(1-methylcyclopropyl)-1H-benzo[d]imidazol-6-yl)oxy)aniline (45e). To a solution of 6-(2,6-dichloro-4-nitrophenoxy)-2-methoxy-1-(1-methylcyclopropyl)-1H-benzo[d]imidazole (45d) (170 mg, 416.43 umol) in EtOH (5 mL) and H₂O (1 mL) was added Fe (116.29 mg, 2.08 mmol) and NH₄Cl (111.37 mg, 2.08 mmol) at 25° C. Then the mixture was stirred at 80° C. for 1 hour. LCMS showed 45d was consumed completely and the desired MS was detected. The suspension was filtered through a pad of Celite and the pad cake was washed with EtOH (10 mL*3). The combined filtrates were extracted with Ethyl acetate (30 mL*2) and H₂O (10 mL). The combined organic phase was washed with brine (10 mL*3), dried with anhydrous Na₂SO₄, filtered and concentrated in vacuum to give 45e. The solid was used directly for the next step without further purification. MS mass calculated for [M+1]⁺ (C₁₈H₁₇Cl₂N₃O₂) requires m/z 378.1, LCMS found m/z 378.1; ¹HNMR (400 MHz, CD₃OD) δ 7.26-7.30 (m, 1H), 6.74-6.81 (m, 2H), 4.06-4.17 (m, 3H), 1.43 (s, 3H), 1.24 (s, 1H), 1.05-1.13 (m, 2H), 0.93-1.00 (m, 2H).

Synthesis of N-(3,5-dichloro-4-((2-methoxy-1-(1-methylcyclopropyl)-1H-benzo[d]imidazol-6-yl)oxy)phenyl)-5-oxo-4,5-dihydro-1,2,4-oxadiazole-3-carboxamide (Example 11). To a solution of 3,5-dichloro-4-((2-methoxy-1-(1-methylcyclopropyl)-1H-benzo[d]imidazol-6-yl)oxy)aniline (45e) (40 mg, 105.75 umol) and 5-oxo-4H-1,2,4-oxadiazole-3-carbonyl chloride (47.11 mg, 317.25 umol) in THF (4 mL) was added TEA (32.10 mg, 317.25 umol, 44.16 uL) at 25° C. Then the mixture was stirred at 25° C. for 0.5 hour. LCMS showed 45e was consumed completely and the desired MS was detected. The mixture was quenched with MeOH (5 mL*3) and stirred at 25° C. for 5 minutes. Then the mixture was concentrated in vacuum. The residue was purified by Prep-HPLC ((FA) column: Welch Xtimate C18 150*25 mm*5 um; mobile phase: [water (0.2%FA)-ACN]). And the obtained solution was diluted with NaHCO₃ (5 mL) and extracted with Ethyl acetate (15 mL*2). The combined organic layers were washed with brine (5 mL*3), dried with anhydrous Na₂SO₄, filtered and concentrated under reduced pressure to give Example 11. MS mass calculated for [M+1]⁺ (C₂₁H₁₇Cl₂N₅O₅) requires m/z 490.1, LCMS found m/z 489.9; ¹HNMR (400 MHz, DMSO-d₆) 610.45-10.55 (m, 1H), 8.11-8.23 (m, 2H), 7.23-7.34 (m, 1H), 6.82-6.96 (m, 1H), 6.45-6.55 (m, 1H), 4.05-4.11 (m, 3H), 1.38 (s, 3H), 0.94-1.09 (m, 4H).

### Example 12

### 2-(3,5-dichloro-4-((2-methoxy-1-(1-methylcyclopropyl)-1H-benzo[d]imidazol-6-yl)oxy)phenyl)-3,5-dioxo-2,3,4,5-tetrahydro-1,2,4-triazine-6-carbonitrile

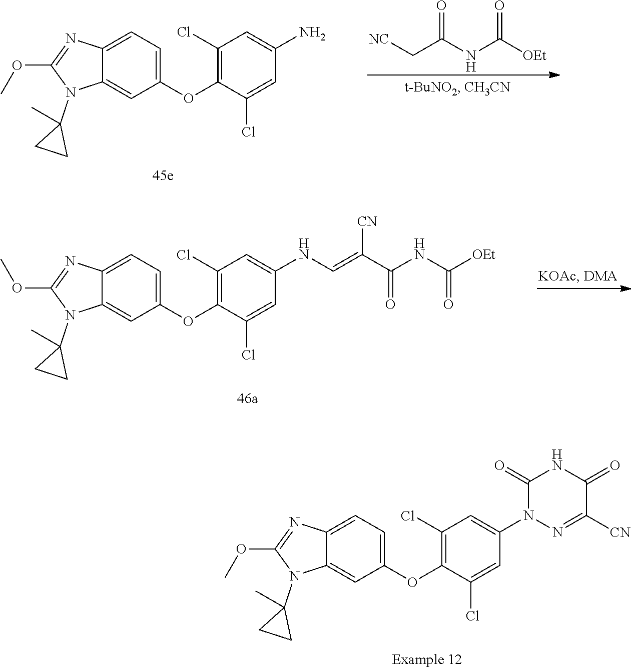 [SMILES: COc1nc2ccc(Oc3c(Cl)cc(N)cc3Cl)cc2n1C1(C)CC1 ; CCOC(=O)NC(=O)CC#N ; CCOC(=O)NC(=O)/C(C#N)=C/Nc1cc(Cl)c(Oc2ccc3nc(OC)n(C4(C)CC4)c3c2)c(Cl)c1 ; COc1nc2ccc(Oc3c(Cl)cc(-n4nc(C#N)c(=O)[nH]c4=O)cc3Cl)cc2n1C1(C)CC1]

Synthesis of (E)-ethyl (2-cyano-2-(2-(3,5-dichloro-4-((2-methoxy-1-(1-methylcyclopropyl)-1H-benzo[d]imidazol-6-yl)oxy)phenyl)hydrazono)acetyl)carbamate (46a). To a mixture of 3,5-dichloro-4-((2-methoxy-1-(1-methylcyclopropyl)-1H-benzo[d]imidazol-6-yl)oxy)aniline (45e) (10 mg, 26.44 umol) and ethyl N-(2-cyanoacetyl)carbamate (12.38 mg, 79.31 umol) in CH₃CN (1 mL) was added t-BuONO (8.18 mg, 79.31 umol, 9.43 uL) at 0° C. Then the mixture was stirred at 0° C. for 1 hour. LCMS showed 45e was consumed completely and the desired MS was detected. The mixture was quenched with MeOH (15 mL) and concentrated in vacuum to give 46a. The solid was used directly in the next step without further purification. MS mass calculated for [M+1]⁺ (C₂₄H₂₂Cl₂N₆O₅) requires m/z 545.1, LCMS found m/z 545.1.

Synthesis of 2-(3,5-dichloro-4-((2-methoxy-1-(1-methylcyclopropyl)-1H-benzo[d]imidazol-6-yl)oxy)phenyl)-3,5-dioxo-2,3,4,5-tetrahydro-1,2,4-triazine-6-carbonitrile (Example 12). To a solution of (E)-ethyl (2-cyano-2-(2-(3,5-dichloro-4-((2-methoxy-1-(1-methylcyclopropyl)-1H-benzo[d]imidazol-6-yl)oxy)phenyl)hydrazono)acetyl)carbamate (46a) (14 mg, 25.67 umol) in DMA (2 mL) was added KOAc (5.04 mg, 51.34 umol). The mixture was stirred at 115° C. for 3 hours. LCMS showed 46a was consumed completely and the desired MS was detected. The mixture was diluted with MeOH (15 mL) and concentrated in vacuum to give a residue. The residue was purified by Prep-HPLC ((FA) column: Xtimate C18 100*30 mm*3 um; mobile phase: [water (0.2%FA)-ACN]). The obtained solution was diluted with NaHCO₃ (5 mL) and extracted with Ethyl acetate (15 mL*2). The combined organic layers were washed with brine (5 mL*3), dried with anhydrous Na₂SO₄, filtered and concentrated under reduced pressure to give Example 12. MS mass calculated for [M+1]⁺ (C₂₂H₁₆Cl₂N₆O₄) requires rn/z 499.1, LCMS found m/z 498.9; ¹HNMR (400 MHz, DMSO-d₆) δ 7.80-7.86 (m, 2H), 7.30 (d, J=8.6 Hz, 1H), 6.94-6.99 (m, 1H), 6.46-6.53 (m, 1H), 4.05-4.13 (m, 3H), 1.40 (s, 3H), 1.03-1.08 (m, 2H), 0.98-1.02 (m, 2H).

### Example 13

### N-(3,5-dichloro-4-((3-(1-methylcyclopropyl)-2-oxo-2,3-dihydro-1H-benzo[d]imidazol-5-yl)oxy)phenyl)-5-oxo-4,5-dihydro-1,2,4-oxadiazole-3-carboxamide

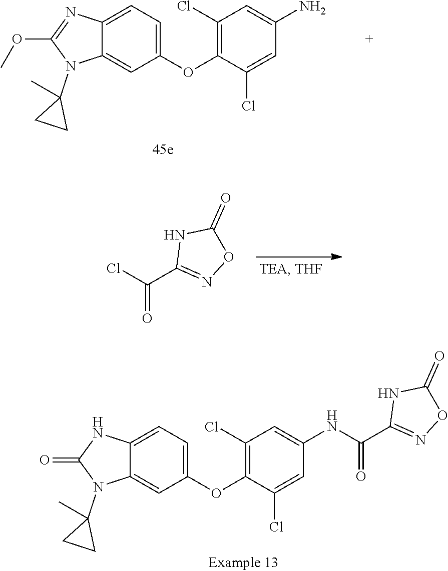 [SMILES: COc1nc2ccc(Oc3c(Cl)cc(N)cc3Cl)cc2n1C1(C)CC1 ; O=C(Cl)c1noc(=O)[nH]1 ; CC1(n2c(=O)[nH]c3ccc(Oc4c(Cl)cc(NC(=O)c5noc(=O)[nH]5)cc4Cl)cc32)CC1]

Synthesis of N-(3,5-dichloro-4-((3-(1-methylcyclopropyl)-2-oxo-2,3-dihydro-1H-benzo[d]imidazol-5-yl)oxy)phenyl)-5-oxo-4,5-dihydro-1,2,4-oxadiazole-3-carboxamide (Example 13). To a solution of 3,5-dichloro-4-((2-methoxy-1-(1-methylcyclopropyl)-1H-benzo[d]imidazol-6-yl)oxy)aniline (45e) (30 mg, 79.31 umol) and 5-oxo-4H-1,2,4-oxadiazole-3-carbonyl chloride (35.33 mg, 237.94 umol) in THF (3 mL) was added TEA (24.08 mg, 237.94 umol, 33.12 uL) at 25° C. Then the mixture was stirred at 25° C. for 0.5 hour. LCMS showed 45e was consumed completely and the desired MS was detected. The mixture was quenched with MeOH (15 mL) and stirred at 25° C. for 5 minutes. Then the mixture was concentrated in vacuum to give a residue. The residue was purified by Prep-HPLC ((FA) column: Welch Xtimate C18 150*25 mm*5 um; mobile phase: [water (0.2%FA)-ACN]) to give Example 13. MS mass calculated for [M+1]⁺ (C₂₀H₁₅Cl₂N₅O₅) requires m/z 476.0, LCMS found m/z 475.9; ¹HNMR (400 MHz, DMSO-d₆) δ11.24-11.34 (m, 1H), 10.59-10.68 (m, 1H), 8.05 (s, 2H), 6.78-6.85 (m, 2H), 6.27 (dd, J=8.50, 2.51 Hz, 1H), 1.35 (s, 3H), 0.88-0.98 (m, 4H).

### Example 14

### 2-(3,5-dichloro-4-((3-(1-methylcyclopropyl)-2-oxo-2,3-dihydro-1H-benzo[d]imidazol-5-yl)oxy)phenyl)-3,5-dioxo-2,3,4,5-tetrahydro-1,2,4-triazine-6-carbonitrile

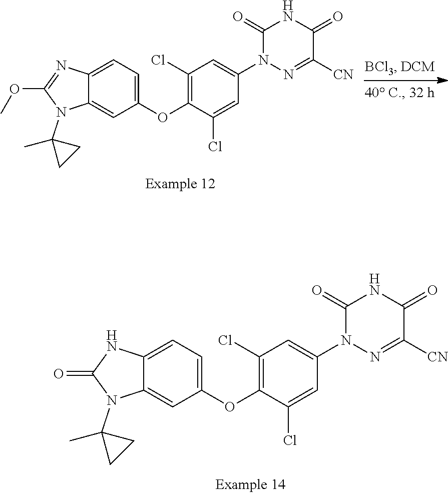 [SMILES: COc1nc2ccc(Oc3c(Cl)cc(-n4nc(C#N)c(=O)[nH]c4=O)cc3Cl)cc2n1C1(C)CC1 ; CC1(n2c(=O)[nH]c3ccc(Oc4c(Cl)cc(-n5nc(C#N)c(=O)[nH]c5=O)cc4Cl)cc32)CC1]

Synthesis of 2-(3,5-dichloro-4-((3-(1-methylcyclopropyl)-2-oxo-2,3-dihydro-1H-benzo[d]imidazol-5-yl)oxy)phenyl)-3,5-dioxo-2,3,4,5-tetrahydro-1,2,4-triazine-6-carbonitrile (Example 14). To a solution of 2-(3,5-dichloro-4-((2-methoxy-1-(1-methylcyclopropyl)-1H-benzo[d]imidazol-6-yl)oxy)phenyl)-3,5-dioxo-2,3,4,5-tetrahydro-1,2,4-triazine-6-carbonitrile (Example 12) (28 mg, 56.08 umol) in DCM (2 mL) was added BCl₃/DCM (1 M, 112.16 ul, 112.16 umol). The mixture was stirred at 40° C. for 32 hours. LCMS and HPLC showed Example 12 was consumed and the desired MS was detected. The mixture was quenched by MeOH (5 mL*3) and then the mixture was concentrated in vacuum to give a residue. The residue was purified by Prep-HPLC (column: Welch Xtimate C18 150*25 mm*5 um; mobile phase: [water (0.2%FA)-ACN]) to give Example 14. MS mass calculated for [M+1]⁺ (C₂₁H₁₄Cl₂N₆O₄) requires m/z 485.0, LCMS found m/z 485.0; ¹H NMR (400 MHz, DMSO-d₆) δ 10.65-10.69 (m, 1H), 7.79-7.83 (m, 2H), 6.82-6.89 (m, 2H), 6.23-6.29 (m, 1H), 1.34-1.39 (m, 3H), 0.95-1.01 (m, 2H), 0.89-0.95 (m, 2H).

### Example 15

### 2-(4-((1-(tert-butyl)-2-methoxy-1H-benzo[d] imidazol-6-yl)oxy)-3,5-dichlorophenyl)-3,5-dioxo-2,3,4,5-tetrahydro-1,2,4-triazine-6-carbonitrile

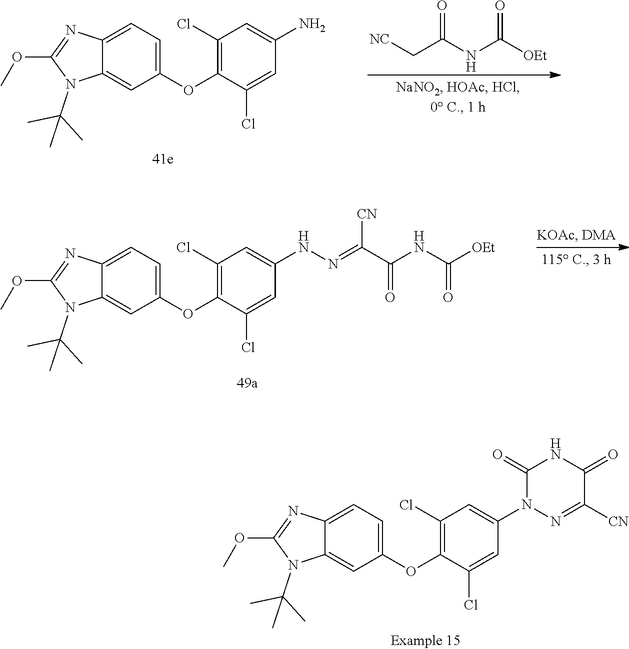 [SMILES: COc1nc2ccc(Oc3c(Cl)cc(N)cc3Cl)cc2n1C(C)(C)C ; CCOC(=O)NC(=O)CC#N ; CCOC(=O)NC(=O)/C(C#N)=N/Nc1cc(Cl)c(Oc2ccc3nc(OC)n(C(C)(C)C)c3c2)c(Cl)c1 ; COc1nc2ccc(Oc3c(Cl)cc(-n4nc(C#N)c(=O)[nH]c4=O)cc3Cl)cc2n1C(C)(C)C]

Synthesis of (E)-ethyl (2-(2-(4-((1-(tert-butyl)-2-methoxy-1H-benzo[d]imidazol-6-yl)oxy)-3,5-dichlorophenyl)hydrazono)-2-cyanoacetyl)carbamate (49a). To a solution of 4-((1-(tert-butyl)-2-methoxy-1H-benzo[d]imidazol-6-yl)oxy)-3,5-dichloroaniline (41e) (50 mg, 131.49 umol) in HOAc (3 mL) and H₂O (1.5 mL) was added ethyl (2-cyanoacetyl)carbamate (102.65 mg, 657.43 umol). Then HCl (1 M, 32.87 uL) was added to the mixture dropwise at 2-4° C. The mixture was stirred at 0° C. for 10 minutes. A solution of NaNO₂ (11.79 mg, 170.93 umol) in H₂O (1.5 mL) was added in the reaction mixture dropwise at 0° C. The solid in the mixture was consumed completely. Then the mixture was stirred at 0° C. for 1 hour. LCMS showed 41e was consumed completely. The reaction mixture was added in water (5 mL) and filtered. The filter cake was diluted with MeOH (10 mL) and concentrated under reduced pressure to give 49a. The crude product was used in the next step without further purification. MS mass calculated for [M+H]⁺ (C₂₄H₂₄Cl₂N₆O₅) requires m/z 547.1, LCMS found m/z 547.1.

Synthesis of 2-(4-((1-(tert-butyl)-2-methoxy-1H-benzo[d]imidazol-6-yl)oxy)-3,5-dichlorophenyl)-3,5-dioxo-2,3,4,5-tetrahydro-1,2,4-triazine-6-carbonitrile (Example 15). To a solution of (E)-ethyl (2-(2-(4-((1-(tert-butyl)-2-methoxy-1H-benzo[d]imidazol-6-yl)oxy)-3,5-dichlorophenyl)hydrazono)-2-cyanoacetyl)carbamate (49a) (50 mg, 91.34 umol) in DMA (5 mL) was added KOAc (17.93 mg, 182.69 umol). The mixture was stirred at 115° C. for 3 hours. LCMS showed 49a was consumed completely. The reaction mixture was diluted with water (5 mL) and extracted with Ethyl acetate (10 mL*3). The combined organic layers were washed with brine (10 mL), dried over Na₂SO₄, filtered and concentrated under reduced pressure to give a residue. The residue was purified by prep-HPLC (column: Xtimate C18 100*30 mm*3 um; mobile phase: [water (0.2%FA)-ACN]) to give Example 15. MS mass calculated for [M+H]⁺ (C₂₂H₁₈Cl₂N₆O₄) requires m/z 501.1, LCMS found m/z 501.0; ¹H NMR (400 MHz, DMSO-d₆) δ 7.81 (s, 2H), 7.30 (d, J=8.6 Hz, 1H), 7.23 (d, J=2.4 Hz, 1H), 6.48 (dd, J=2.4, 8.6 Hz, 1H), 4.07 (s, 3H), 3.33 (s, 72H), 1.66-1.70 (m, 9H).

### Example 16

### 2-(4-((3-(tert-butyl)-2-oxo-2,3-dihydro-1H-benzo[d]imidazol-5-yl)oxy)-3,5-dichlorophenyl)-3,5-dioxo-2,3,4,5-tetrahydro-1,2,4-triazine-6-carbonitrile

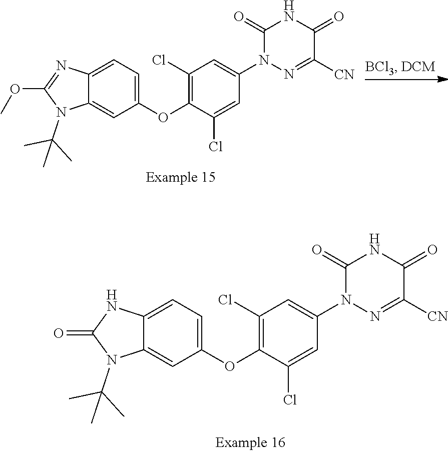 [SMILES: COc1nc2ccc(Oc3c(Cl)cc(-n4nc(C#N)c(=O)[nH]c4=O)cc3Cl)cc2n1C(C)(C)C ; CC(C)(C)n1c(=O)[nH]c2ccc(Oc3c(Cl)cc(-n4nc(C#N)c(=O)[nH]c4=O)cc3Cl)cc21]

Synthesis of 2-(4-((3-(tert-butyl)-2-oxo-2,3-dihydro-1H-benzo[d]imidazol-5-yl)oxy)-3,5-dichlorophenyl)-3,5-dioxo-2,3,4,5-tetrahydro-1,2,4-triazine-6-carbonitrile (Example 16). A mixture of 2-(4-((1-(tert-butyl)-2-methoxy-1H-benzo[d]imidazol-6-yl)oxy)-3,5-dichlorophenyl)-3,5-dioxo-2,3,4,5-tetrahydro-1,2,4-triazine-6-carbonitrile (Example 15) (10 mg, 19.95 umol) in DCM (3 mL) was added BCl₃ (4.67 mg, 39.89 umol, 5.19 uL), then degassed and purged with N₂ 3 times, and then the mixture was stirred at 40° C. for 24 hours under N2 atmosphere. LCMS showed the reaction was completed and the desired MS was detected. The reaction mixture was quenched by addition MeOH (2 mL) at 20° C., and then concentrated under reduced pressure. The residue was purified by prep-HPLC (column: Waters Xbridge BEH C18 100*30 mm*10 um; mobile phase: [water (10 mM NH₄HCO₃)-ACN]) to give Example 16. MS mass calculated for [M+1]⁺ (C₂₁H₁₆Cl₂N₆O₄) requires m/z 487.1, LCMS found m/z 487.0; ¹H NMR (400 MHz, CD₃OD) δ 7.77 (s, 2H), 7.12 (d, J=2.2 Hz, 1H), 6.91 (d, J=8.6 Hz, 1H), 6.44 (dd, J=8.4, 2.0 Hz,1H), 1.73-1.77 (m, 9H).

### Example 17

### N-(3,5-dichloro-4-((3-(1-(difluoromethyl)cyclopropyl)-2-oxo-2,3-dihydro-1H-benzo[d]imidazol-5-yl)oxy)phenyl)-5-oxo-4,5-dihydro-1,2,4-oxadiazole-3-carboxamide

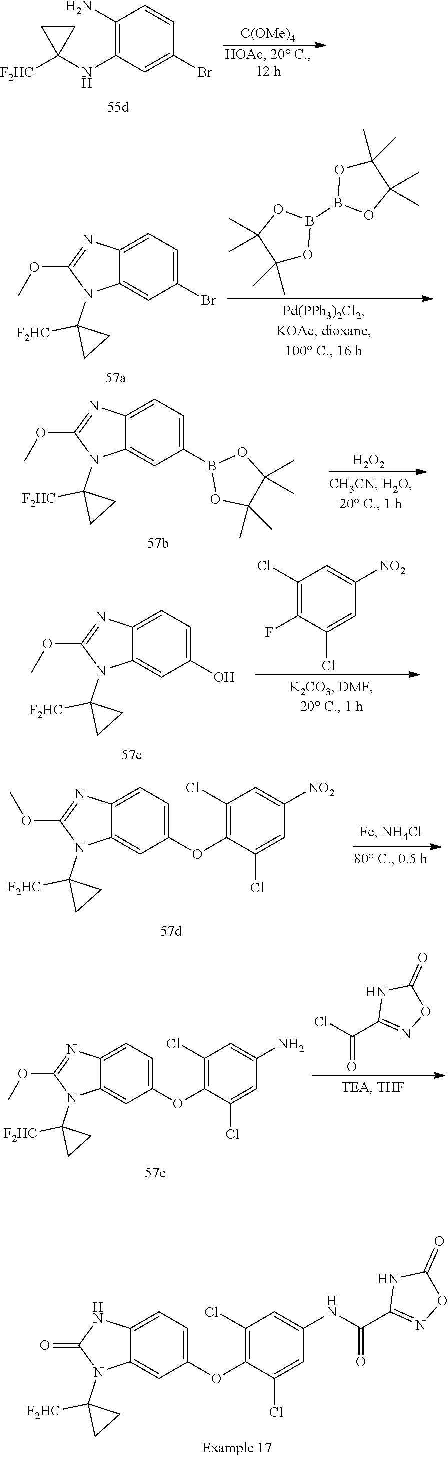

Synthesis of 6-bromo-1-(1-(difluoromethyl)cyclopropyl)-2-methoxy-1H-benzo[d]imidazole (57a). To a solution of 5-bromo-N1-(1-(difluoromethyl)cyclopropyl)benzene-1,2-diamine (55d) (122 mg, 440.26 umol) in AcOH (3 mL) was added tetramethoxymethane (239.77 mg, 1.76 mmol). The mixture was stirred at 20° C. for 12 hours. TLC and LCMS showed 55d was consumed completely and the desired MS was detected. The reaction mixture was concentrated under reduced pressure to remove AcOH. The residue was diluted with saturated NaHCO₃ (10 mL) and extracted with EtOAc (10 mL*2). The combined organic layers were washed with brine (10 mL), dried over anhydrous Na₂SO₄, filtered and concentrated under reduced pressure. The residue was purified by Prep-TLC (SiO₂, Petroleum ether: Ethyl acetate) to give 57a. MS mass calculated for [M+1]⁺ (C₁₂H₁₁BrF₂N₂O) requires m/z 317.0, LCMS found m/z 317.1; 1 H NMR (400 MHz, CDCl₃) δ 7.46 (s, 1H), 7.39 (d, J=8.6 Hz, 2H), 7.32-7.28 (m, 1H), 6.02-5.71 (m, 1H), 4.21 (s, 3H), 1.63-1.55 (m, 3H), 1.54-1.49 (m, 2H), 1.32 (br s, 2H).

Synthesis of 1-(1-(difluoromethyl)cyclopropyl)-2-methoxy-6-(4,4,5,5-tetramethyl-1,3,2-dioxab-orolan-2-yl)-1H-benzo[d]imidazole (57b). A mixture of 6-bromo-1-(1-(difluoromethyl)cyclopropyl)-2-methoxy-1H-benzo[d]imidazole (57a) (103 mg, 324.79 umol), BPD (247.43 mg, 974.37 umol), Pd (PPh₃)₂Cl₂ (22.80 mg, 32.48 umol) and AcOK (159.37 mg, 1.62 mmol) in dioxane (3 mL) was degassed and purged with N₂ 3 times, and then the mixture was stirred at 100° C. for 16 hours under N₂ atmosphere. LCMS showed 57a was consumed completely and the desired MS was detected. The suspension was filtered through a pad of Celite and the pad cake was washed with EtOAc (5 mL*3). The combined filtrates were diluted with brine (10 mL) and extracted with EtOAc (10 mL*2). The combined organic layers dried over Na₂SO₄, filtered and concentrated under reduced pressure. The residue was purified by Prep-TLC (SiO₂, Petroleum ether: Ethyl acetate) to give 57b. MS mass calculated for [M+1]⁺ (C₁₈H₂₃BF₂N₂O₃) requires m/z 365.2, LCMS found m/z 365.2; ¹H NMR (400 MHz, CDCl₃) δ 7.74 (s, 1H), 7.69 (dd, J=0.8, 8.0 Hz, 1H), 7.53 (d, J=8.0 Hz, 1H), 6.09-5.80 (m, 1H), 4.23 (s, 3H), 1.57-1.52 (m, 2H), 1.37 (s, 12H), 1.35 (br s, 2H).

Synthesis of 1-(1-(difluoromethyl)cyclopropyl)-2-methoxy-1H-benzo[d]imidazol-6-ol (57c). To a solution of 1-(1-(difluoromethyl)cyclopropyl)-2-methoxy-6-(4,4,5,5-tetramethyl-1,3,2-dioxab-orolan-2-yl)-1H-benzo[d]imidazole (57b) (99.5 mg, 273.21 umol) in ACN (2 mL) was added a solution of NH₄HCO₃ (21.60 mg, 273.21 umol, 22.50 uL) in H₂O (1 mL) at 20° C. Then H₂O₂ (61.94 mg, 546.41 umol, 52.50 uL, 30% purity) was added in the reaction mixture dropwise at 20° C. The resulting mixture was stirred at 20° C. for 1 hour. TLC indicated 57b was consumed completely and one new spot was formed. The mixture was poured into a saturated solution of NaHSO₃ (3 mL) and stirred for 10 minutes. The aqueous phase was extracted with ethyl acetate (10 mL*2). The combined organic phase was washed with brine 10 mL, dried with anhydrous Na₂SO₄, filtered and concentrated in vacuum to give 57c. The crude product was used in the next step without further purification.

Synthesis of 6-(2,6-dichloro-4-nitrophenoxy)-1-(1-(difluoromethyl)cyclopropyl)-2-methoxy-1H-benzo[d]imidazole (57d). To a solution of 1-(1-(difluoromethyl)cyclopropyl)-2-methoxy-1H-benzo[d]imidazol-6-ol (57c) (68.00 mg, 267.48 umol) and 1,3-dichloro-2-fluoro-5-nitro-benzene (58.97 mg, 280.85 umol) in DMF (3 mL) was added K₂CO₃ (55.45 mg, 401.21 umol). The mixture was stirred at 20° C. for 1 hour. TLC indicated 57c was consumed completely and the desired spot was found. The reaction mixture was diluted with water (10 mL) and extracted with EtOAc (10 mL*2). The combined organic layers were washed with brine (10 mL), dried over anhydrous Na₂SO₄, filtered and concentrated under reduced pressure. The residue was purified by Prep-TLC (Ethyl acetate: Petroleum ether) to give 57d. 1 H NMR (400 MHz, CDCl₃) δ 8.32 (s, 2H), 7.40 (d, J=8.8 Hz, 1H), 6.96 (d, J=2.2 Hz, 1H), 6.57 (dd, J=2.4, 8.6 Hz, 1H), 6.02-5.71 (m, 1H), 4.20 (s, 3H), 1.53-1.45 (m, 2H), 1.35-1.28 (m, 2H).

Synthesis of 3,5-dichloro-4-((1-(1-(difluoromethyl)cyclopropyl)-2-methoxy-1H-benzo[d]imidazo-1-6-yl)oxy)aniline (57e). A mixture of 6-(2,6-dichloro-4-nitrophenoxy)-1-(1-(difluoromethyl)cyclopropyl)-2-methoxy-1H-benzo[d]imidazole (57d) (55 mg, 123.81 umol), Fe (34.57 mg, 619.07 umol), NH₄Cl (33.11 mg, 619.07 umol) in H₂O (1 mL) and MeOH (5 mL) was degassed and purged with N₂ 3 times, and then the mixture was stirred at 80° C. for 0.5 hours under N₂ atmosphere. TLC indicated 57d was consumed completely and one new spot formed. The suspension was filtered through a pad of Celite and the pad cake was washed with MeOH (5 mL*2). The combined filtrates were concentrated to dryness, and then diluted with water (10 mL) and extracted with EtOAc (10 mL*2). The combined organic layers were washed with brine (10 mL), dried over anhydrous Na₂SO₄, filtered and concentrated under reduced pressure. The residue was purified by Prep-TLC (SiO₂, Petroleum ether: Ethyl acetate) to give 57e. ¹H NMR (400 MHz, CDCl₃) δ 7.38 (d, J=8.6 Hz, 1H), 6.98-6.90 (m, 1H), 6.76-6.70 (m, 2H), 6.62 (dd, J=2.4, 8.6 Hz, 1H), 6.05-6.02 (m, 1H), 5.91-5.88 (m, 1H), 5.77-5.74 (m, 1H), 4.18 (s, 3H), 1.51-1.44 (m, 2H), 1.29 (br s, 2H).

Synthesis of N-(3,5-dichloro-4-((3-(1-(difluoromethyl)cyclopropyl)-2-oxo-2,3-dihydro-1H-benzo-[d]imidazol-5-yl)oxy)phenyl)-5-oxo-4,5-dihydro-1,2,4-oxadiazole-3-carboxamide (Example 17). To a solution of 5-oxo-4H-1,2,4-oxadiazole-3-carbonyl chloride (10.76 mg, 72.42 umol) (1.5 M in THF, assume the previous step 100% yield) in THF (10 mL) was added dropwise a solution of TEA (24.43 mg, 241.41 umol, 33.60 uL) and 3,5-dichloro-4-((1-(1-(difluoromethyl)cyclopropyl)-2-methoxy-1H-benzo[d]imidazo-1-6-yl)oxy)aniline (57e) (20 mg, 48.28 umol) in THF (10 mL) at 20° C. over 10 minutes. After addition, the mixture was stirred at this temperature for 30 minutes. TLC and LCMS showed 57e was consumed completely and the desired MS was detected. The mixture was diluted with H₂O (10 mL) and extracted with EtOAc (15 mL*2). The combined organic layers were washed with brine 10 mL, dried over anhydrous Na₂SO₄, filtered and concentrated under reduced pressure. The residue was checked by HPLC and then purified by Prep-HPLC (column: Phenomenex Luna C18 200*40 mm*10 um; mobile phase: [water (0.2%FA)-ACN]) to give Example 17. MS mass calculated for [M+1]⁺ (C₂₀H₁₃Cl₂F₂N₅O₅) requires m/z 512.3, LCMS found m/z 512.0; ¹H NMR (400 MHz, CD₃OD) δ 7.96 (s, 2H), 6.96 (d, J=8.6 Hz, 1H), 6.80 (s, 1H), 6.54-6.49 (m, 1H), 6.03-5.72 (m, 1H), 1.45 (br s, 2H), 1.30 (br s, 2H).

### Example 18

### 2-(3,5-dichloro-4-((3-(1-(difluoromethyl)cyclopropyl)-2-oxo-2,3-dihydro-1H-benzo[d]imidazol-5-yl)oxy)phenyl)-3,5-dioxo-2,3,4,5-tetrahydro-1,2,4-triazine-6-carbonitrile

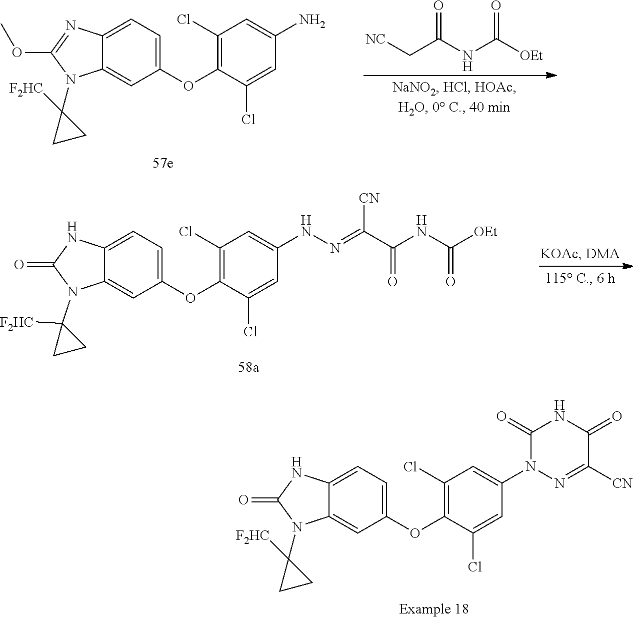 [SMILES: COc1nc2ccc(Oc3c(Cl)cc(N)cc3Cl)cc2n1C1(C(F)F)CC1 ; CCOC(=O)NC(=O)CC#N ; CCOC(=O)NC(=O)/C(C#N)=N/Nc1cc(Cl)c(Oc2ccc3[nH]c(=O)n(C4(C(F)F)CC4)c3c2)c(Cl)c1 ; N#Cc1nn(-c2cc(Cl)c(Oc3ccc4[nH]c(=O)n(C5(C(F)F)CC5)c4c3)c(Cl)c2)c(=O)[nH]c1=O]

Synthesis of (E)-ethyl (2-cyano-2-(2-(3,5-dichloro-4-((3-(1-(difluoromethyl)cyclopropyl)-2-oxo-2,3-dihydro-1H-benzo[d]imidazol-5-yl)oxy)phenyl)hydrazono)acetyl)carbamate (58a). To a solution of 3,5-dichloro-4-((1-(1-(difluoromethyl)cyclopropyl)-2-methoxy-1H-benzo[d]imidazol-6-yl)oxy)aniline (57e) (10 mg, 24.14 umol) and ethyl (2-cyanoacetyl)carbamate (18.85 mg, 120.71 umol) in AcOH (2 mL) was added dropwise HCl (1 M, 6.04 uL) at 0° C. After addition, the mixture was stirred at this temperature for 10 minutes, and then NaNO₂ (2.17 mg, 31.38 umol) in H₂O (1 mL) was added dropwise at 0° C. The resulting mixture was stirred at 0° C. for 0.5 hours. LCMS showed 57e was consumed completely and the desired MS was detected. The reaction mixture was diluted with saturated NaHCO₃ (10 mL) and extracted with EtOAc (10 mL*2). The combined organic layers were washed with brine (10 mL), dried over anhydrous Na₂SO₄, filtered and concentrated under reduced pressure to give 58a. MS mass calculated for [M+1]⁺ (C₂₃H₁₈C₁₂F₂N₆O₅) requires m/z 567.1, LCMS found m/z 567.0. The crude product was used in the next step without further purification.

Synthesis of 2-(3,5-dichloro-4-((3-(1-(difluoromethyl)cyclopropyl)-2-oxo-2,3-dihydro-1H-benzo[d]imidazol-5-yl)oxy)phenyl)-3,5-dioxo-2,3,4,5-tetrahydro-1,2,4-triazine-6-carbonitrile (Example 18). A mixture of (E)-ethyl (2-cyano-2-(2-(3,5-dichloro-4-((3-(1-(difluoromethyl)cyclopropyl)-2-oxo-2,3-dihydro-1H-benzo[d]imidazol-5-yl)oxy)phenyl)hydrazono)acetyl)carbamate (58a) (21.47 mg, 37.84 umol) and AcOK (7.43 mg, 75.69 umol) in DMA (1 mL) was degassed and purged with N₂ 3 times, and then the mixture was stirred at 115° C. for 6 hours under N₂ atmosphere. LCMS showed 58a was consumed completely and the desired product was detected. The reaction mixture was diluted with water (10 mL) and extracted with EtOAc (10 mL*3). The combined organic layers were washed with brine (10 mL), dried over anhydrous Na₂SO₄, filtered and concentrated under reduced pressure. The residue was purified by Prep-HPLC (column: Phenomenex Luna C18 200*40 mm*10 um; mobile phase: [water (0.2%FA)-ACN]) to give Example 18. MS mass calculated for [M+1]⁺ (C₂₁H₁₂Cl₂F₂N₆O₄) requires m/z 521.0, LCMS found m/z 521.0; 1 H NMR (400 MHz, CD₃OD) δ 7.78 (s, 2H), 6.95 (d, J=8.8 Hz, 1H), 6.84 (s, 1H), 6.50 (dd, J=2.6, 8.6 Hz, 1H), 6.00-5.72 (m, 1H), 4.85 (s, 86H), 3.34-3.28 (m, 27H), 1.44 (br s, 2H), 1.28 (br s, 2H).

### Example 19

### N-(3,5-dichloro-4-((3-methyl-2-oxo-2,3-dihydro-1H-benzo[d]imidazol-5-yl)oxy)phenyl)-5-oxo-4,5-dihydro-1,2,4-oxadiazole-3-carboxamide

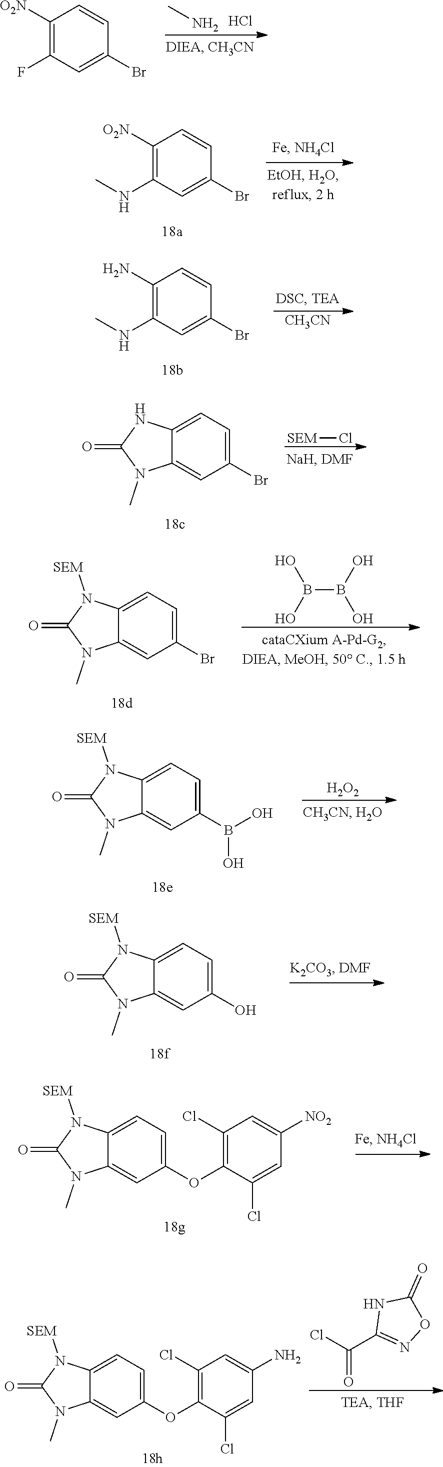

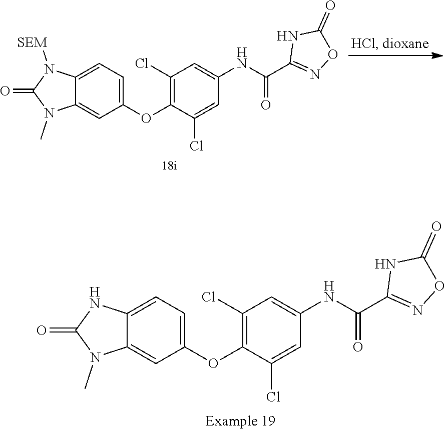 [SMILES: Cn1c(=O)n(COCC[Si](C)(C)C)c2ccc(Oc3c(Cl)cc(NC(=O)c4noc(=O)[nH]4)cc3Cl)cc21 ; Cn1c(=O)[nH]c2ccc(Oc3c(Cl)cc(NC(=O)c4noc(=O)[nH]4)cc3Cl)cc21]

Synthesis of 5-bromo-N-methyl-2-nitroaniline (18a). To a solution of 4-bromo-2-fluoro-1-nitrobenzene (2 g, 9.09 mmol) and methylamine (2.47 g, 36.53 mmol, HCl) in CH₃CN (50 mL) was added DIEA (5.87 g, 45.46 mmol, 7.92 mL). Then the mixture was stirred at 60° C. for 12 hours. TLC showed the reaction was completed. The mixture was concentrated in vacuum. The residue was extracted with EtOAc (50 mL+20 mL) and H₂O (20 mL). The combined organic layer was washed with brine (10 mL), dried over Na₂SO₄, filtered and concentrated in vacuum to give 18a.

Synthesis of 5-bromo-N1-methylbenzene-1,2-diamine (18b). To a solution of 5-bromo-N-methyl-2-nitroaniline (18a) (1.8 g, 7.79 mmol) in EtOH (30 mL) and H₂O (10 mL) was added NH₄Cl (2.08 g, 38.95 mmol) and iron powder (2.18 g, 38.95 mmol). Then the mixture was stirred at 80° C. for 16 hours. LCMS showed the reaction was completed, and desired MS was detected. The mixture was filtered and the filtration was concentrated in vacuum to give 18b. MS mass calculated for [M+1]⁺ (C₇H₉BrN₂) requires m/z 201.0, LCMS found m/z 201.0.

Synthesis of 6-bromo-1-methyl-1H-benzo[d]imidazol-2(3H)-one (18c). To a solution of 5-bromo-N1-methylbenzene-1,2-diamine (18b) (1.2 g, 5.97 mmol) in CH₃CN (30 mL) was added TEA (1.81g, 17.90 mmol, 2.49 mL) and DSC (1.68 g, 6.57 mmol). Then the mixture was stirred at 20° C. for 16 hours. TLC showed the reaction was completed. The mixture was concentrated in vacuum. The residue was diluted in H₂O (15 mL) and EtOAc (15 mL). The mixture was filtrated to collect solid. The solid was was extracted with EtOAc (5 mL*5) and dried over in vacuum to give 18c.

Synthesis of 5-bromo-3-methyl-1-((2-(trimethylsilyl)ethoxy)methyl)-1H-benzo[d]imidazol-2(3H)-one (18d). To a mixture of 6-bromo-1-methyl-1H-benzo[d]imidazol-2(3H)-one (18c) (1.1 g, 4.84 mmol) in DMF (15 mL) was added NaH (213.14 mg, 5.33 mmol, 60% purity) at 20° C. Then the mixture was stirred at 20° C. for 10 min. Then SEM-Cl (888.46 mg, 5.33 mmol) was added in the mixture by dropwise. Then the mixture was stirred at 20° C. for 10 minutes. TLC showed the starting material was consumed, and one new spot was formed. The mixture was added in H₂O (45 mL) and extracted with EtOAc (20 mL*2). The combined organic layer was washed with brine (10 mL*2), dried over Na₂SO₄, filtered and concentrated in vacuum. The residue was purified by column silicagel chromatography (petroleum ether: ethyl acetate) to give 18d. ¹H NMR (400 MHz, CHLOROFORM-d) δ ppm 7.27 (s, 1 H), 7.23 (dd, J=8.4, 1.8 Hz, 1 H) ,7.13 (d, J=1.8 Hz, 1 H), 7.04 (d, J=8.4 Hz, 1 H), 5.30 (s, 2 H), 3.55-3.64 (m, 2 H), 3.41 (s, 3 H), 0.95-0.97 (m, 1 H), 0.88-0.94 (m, 2 H), -0.05-0.00 (m, 8 H).

Synthesis of (3-methyl-2-oxo-1-((2-(trimethylsilyl)ethoxy)methyl)-2,3-dihydro-1H-benzo[d]imidazol-5-yl)boronic acid (18e). To a mixture of 5-bromo-3-methyl-1-((2-(trimethylsilyl)ethoxy)methyl)-1H-benzo[d]imidazol-2(3H)-one (18d) (200 mg, 559.73 umol) and hypoboric acid (150.54 mg, 1.68 mmol) in MeOH (5 mL) was added DIEA (217.02 mg, 1.68 mmol, 292.48 uL) and cataCXium A-Pd-G2 (3.74 mg, 5.60 umol) under N₂. The mixture was stirred at 50° C. for 1.5 hours. LCMS showed the 18d was consumed completely and desired MS was detected. The reaction mixture was filtered and concentrated under reduced pressure to give a residue to give 18e. MS mass calculated for [M+1]⁺ (C₁₄H₂₃BN₂O₄Si) requires m/z 323.2, LCMS found m/z 323.1

Synthesis of 5-hydroxy-3-methyl-1-((2-(trimethylsilyl)ethoxy)methyl)-1H-benzo[d]imidazol-2(3H)-one (18f). To a mixture of (3-methyl-2-oxo-1-((2-(trimethylsilyl)ethoxy)methyl)-2,3-dihydro-1H-benzo[d]imidazol-5-yl)boronic acid (18e) (130 mg, 403.43 umol) in CH₃CN (2 mL) was added NH₄HCO₃ (31.89 mg, 403.43 umol, 33.22 uL) in H₂O (1 mL) and H₂O₂ (91.48 mg, 806.85 umol, 77.53 uL, 30% purity) under N₂. The mixture was stirred at 25° C. for 2 hours. The reaction mixture was poured into NaHSO₃ (10 mL). The aqueous phase was extracted with ethyl acetate (30 mL*2). The combined organic phase was washed with brine (20 mL*2), dried with anhydrous Na₂SO₄, filtered and concentrated in vacuum to give 18f. MS mass calculated for [M+1]⁺ (C₁₄H₂₂N₂O₃Si) requires m/z 295.1, LCMS found m/z 295.2; 1 HNMR (400 MHz, CDCl₃) δ 8.03 (s, 1H), 6.99 (s, 1H), 6.68-6.50 (m, 2H), 5.31-5.27 (m, 2H), 3.63-3.56 (m, 2H), 3.40-3.36 (m, 3H), 2.99-2.96 (m, 2H), 2.90 (s, 2H), 2.10 (s, 1H), 1.02-0.82 (m, 3H), −0.02-−0.05 (m, 9H).

Synthesis of 5-(2,6-dichloro-4-nitrophenoxy)-3-methyl-1-((2-(trimethylsilyl)ethoxy)methyl)-1H-benzo[d]imidazol-2(3H)-one (18g). To a mixture of 5-hydroxy-3-methyl-1-((2-(trimethylsilyl)ethoxy)methyl)-1H-benzo[d]imidazol-2(3H)-one (18f) (130 mg, 441.55 umol) in DMF (3 mL) was added K₂CO₃ (91.54 mg, 662.32 umol) and 1,3-dichloro-2-fluoro-5-nitro-benzene (101.99 mg, 485.70 umol) under N₂. The mixture was stirred at 20° C. for 1 hour. LCMS showed the 18f was consumed completely and desired MS was detected. TLC indicated the starting material was consumed completely and many new spots were formed. The residue was poured into water (5 mL). The aqueous phase was extracted with ethyl acetate (15 mL*3). The combined organic phase was washed with brine (10 mL*2), dried with anhydrous Na₂SO₄, filtered and concentrated in vacuum. The residue was purified by prep-TLC (SiO₂, petroleum ether/ethyl acetate) to give 18g. MS mass calculated for [M+1]⁺ (C₂₀H₂₃Cl₂N₃O₅Si) requires m/z 484.1, LCMS found m/z 484.1; ¹H NMR (400 MHz, CDCl₃) δ 8.34 (s, 2H), 7.27 (s, 1H), 7.04 (d, J=8.4 Hz, 1H), 6.67-6.58 (m, 1H), 6.48-6.39 (m, 1H), 5.29 (s, 2H), 3.65-3.57 (m, 2H), 3.40 (s, 3H), 1.57 (s, 2H), 1.02-0.82 (m, 2H), -0.03 (s, 9H).

Synthesis of 5-(4-amino-2,6-dichlorophenoxy)-3-methyl-1-((2-(trimethylsilyl)ethoxy)methyl)-1H-benzo[d]imidazol-2(3H)-one (18h). To a mixture of 5-(2,6-dichloro-4-nitrophenoxy)-3-methyl-1-((2-(trimethylsilyl)ethoxy)methyl)-1H-benzo[d]imidazol-2(3H)-one (18g) (100 mg, 206.44 umol) in EtOH (4 mL) was added Fe (57.64 mg, 1.03 mmol) and NH₄Cl (55.21 mg, 1.03 mmol) in H₂O (1 mL) under N₂. The mixture was stirred at 80° C. for 2 hours. LCMS showed the starting material was consumed completely and one main peak with desired MS was detected. The reaction mixture was filtered and concentrated under reduced pressure to give a residue. The residue was diluted with ethyl acetate (10 mL) and water (10 mL). The aqueous phase was extracted with ethyl acetate (20 mL*2). The combined organic phase was washed with brine (15 mL*2), dried with anhydrous Na₂SO₄, filtered and concentrated in vacuum to give 18h. MS mass calculated for [M+1]⁺ (C₂₀H₂₅Cl₂N₃O₃Si) requires m/z 454.1, LCMS found m/z 454.1; ¹HNMR (400 MHz, CDCl₃) δ 7.04-6.98 (m, 1H), 6.72 (s, 2H), 6.60-6.58 (m, 1H), 6.54-6.50 (m, 1H), 5.28 (s, 2H), 3.71-3.50 (m, 2H), 3.38 (s, 3H), 1.02-0.82 (m, 2H), -0.02 (s, 9H).

Synthesis of N-(3,5-dichloro-44(3-methyl-2-oxo-14(2-(trimethylsilyl)ethoxy)methyl)-2,3-dihydro-1H-benzo[d]imidazol-5-yl)oxy)phenyl)-5-oxo-4,5-dihydro-1,2,4-oxadiazole-3-carboxamide (18i). To a mixture of 5-(4-amino-2,6-dichlorophenoxy)-3-methyl-1-((2-(trimethylsilyl)ethoxy)methyl)-1H-benzo[d]imidazol-2(3H)-one (18h) (20 mg, 44.01 umol) and in THF (1.5 mL) was added TEA (13.36 mg, 132.04 umol, 18.38 uL) and 5-oxo-4H-1,2,4-oxadiazole-3-carbonyl chloride (6.54 mg, 44.01 umol) under N₂. The mixture was stirred at 20° C. for 20 min. LCMS showed the 18h was consumed completely and one main peak with desired MS was detected. The reaction mixture was concentrated under reduced pressure to give a residue. The residue was purified by Prep-HPLC (column: Waters Xbridge BEH C18 100*30 mm*10 um; mobile phase: [water (10 mM NH₄HCO₃)-MeCN]) to give 18i. MS mass calculated for [M+1]⁺ (C₂₃H₂₅Cl₂N₅O₆Si) requires m/z 566.1, LCMS found m/z 566.2; ¹H NMR (400 MHz, METHANOL-d₄) δ 8.00-7.92 (m, 2H), 7.19-7.08 (m, 1H), 6.81-6.72 (m, 1H), 6.57-6.50 (m, 1H), 5.33-5.28 (m, 2H), 3.66-3.57 (m, 2H), 3.39-3.38 (m, 3H), 0.96-0.82 (m, 2H), −0.01-−0.07 (m, 9H).

Synthesis of N-(3,5-dichloro-4-((3-methyl-2-oxo-2,3-dihydro-1H-benzo[d]imidazol-5-yl)oxy)phenyl)-5-oxo-4,5-dihydro-1,2,4-oxadiazole-3-carboxamide (Example 19). To a mixture of N-(3,5-dichloro-4-((3-methyl-2-oxo-1-((2-(trimethylsilyl)ethoxy)methyl)-2,3-dihydro-1H-benzo[d]imidazol-5-yl)oxy)phenyl)-5-oxo-4,5-dihydro-1,2,4-oxadiazole-3-carboxamide (18i) (8.5 mg, 15.01 umol) in dioxane (0.5 mL) was added HCl (2 mL) under N₂. The mixture was stirred at 65° C. for 6 hours. LCMS showed the 18i was consumed. The reaction mixture was concentrated under reduced pressure to give a residue. The residue was purified by Prep-HPLC (column: Waters Xbridge BEH C18 100*30 mm*10 um; mobile phase: [water (10 mM NH₄HCO₃)—MeCN]) to give Example 19. MS mass calculated for [M+1]⁺ (C₁₇H₁₁Cl₂N₅O₅) requires m/z 436.0, LCMS found m/z 435.9; 1 H NMR (400 MHz, MeOH-d₄) δ 8.02-7.94 (m, 2H), 6.98 (d, J=8.6 Hz, 1H), 6.70 (d, J=2.4 Hz, 1H), 6.51 (dd, J=2.4, 8.6 Hz, 1H), 4.77 (s, 1H), 3.35 (s, 3H).

It is understood that compounds disclosed herein are synthesized using the General Synthetic Schemes or using the experimental procedures as described above and the steps involved in the synthetic routes are clearly familiar to those skilled in the art, wherein the substituents described in the formulae disclosed herein can be varied with a choice of appropriate starting materials and reagents utilized in the steps presented.

### Biological Example: Biological Screening

### Example B1

### Time-Resolved Fluorescence Resonance Energy Transfer (TR-FRET) Assay for Thyroid Hormone Receptor Agonist Screening

LanthaScreen™ TR-FRET Thyroid Receptor alpha Coactivator Assay kit (ThermoFisher) and LanthaScreen™ TR-FRET Thyroid Receptor beta Coactivator Assay kit (ThermoFisher) were used for agonist compound screening. Compounds in DMSO were diluted using ECHO Liquid Handler (Labcyte Inc.) into 384 plates in 10-point 3-fold series in duplicate (5 micro M final top concentration). Buffer C (ThermoFisher) was added to each well before the 4× mixture of fluorescein-SCR2-2 coactivator (200 nM final concentration), Terbium-labeled anti-GST antibody (2 nM final concentration), and TR alpha-LBD (0.4 nM final concentration) or TR beta-LBD (1.0 nM final concentration) was added. After 2 hour incubation at room temperature in dark, the TR-FRET signal was measured on an EnVision plate reader (PerkinElmer) with excitation at 340 nm and dual emission readout at 495 and 520 nm with the delay time of 100 micro second and the integration time of 200 micro second. The ratio of emission signal at 520 and at 495 was used to calculate EC₅₀ using GraphPad Prism (GraphPad Software). In every batch of compound screening, T3 (L-3,3′,5-Triiodothyronine sodium salt, >95%) (Calbiochem) was used as reference compound. The EC₅₀ of T3 measured were within 3-fold of the reference value provided by the assay kit manufacturer (ThermoFisher Scientific). The Z′ factors measured in every batch of screening using T3 as high percent effect (HPE) control and 0.5% DMSO as zero percent effect (ZPE) control were in the range of 0.5 to 0.8. Compounds' THR-beta selectivity values are derived from T3-selectivity normalized data. Data obtained using the TR-FRET assay for certain compounds disclosed herein are listed in Table 2.

TABLE 2

| | EC₅₀ THRß- FRET | EC₅₀ THRα- FRET | |
|---|---|---|---|
| Example | [nM] | [nM] | THRß-Selectivity |
| 19 | 73.6 | 244.6 | 14.1 |

### Example B2

### THR/RXR Heterodimer Assay for Thyroid Hormone Receptor Agonist Screening

Test compounds were prepared as 10 mM DMSO stock solutions. The stock solution (45 uL) was transferred to a 384-well assay plate, and 3-fold, 10-point dilutions were performed by transferring 15 μL of the compound solution into 30 μL DMSO using TECAN (EVO200) liquid handler. The compound solutions (200 nL, serially diluted) and the positive control triiodothyronine (T3) (100 nL) were transferred to an assay plate using ECHO550. Next, H6-THR-α (150.64 uM, 10 μL) or H6-THR-β (32.57 uM, 10 μL) in binding buffer (50 mM HEPES, pH 7.0, 1 mM DTT, 0.05% NP40, 0.2 mg/mL BSA) was mixed with retinoid X receptor alpha (RxRα) (146.76 uM, 10 μL) in binding buffer, and transferred to the 384-well assay plate containing T3 or compound solution. After incubation at 37° C. for 30 min, biotin-GRIP1 peptide (3262.1 uM, 10 μL) in binding buffer and 5% DMSO was added to the 384-well assay plate and incubated at 37° C. for 30 min. A solution (10 μL) containing europium-conjugated anti-hexa(His) antibody (0.625 uM) and APC-conjugated streptavidin (1.18 uM) in buffer (50 mM Tris, pH 7.4, 100 mM NaCl, and 0.2 mg/mL BSA) was then added to the 384-well assay plate and incubated at 25° C. for 60 min. The assay plate was read using Envision (PerkinElmer), using T3 as the positive control for both THR-β/RXR-α and THR-α/RXR-α activity. DMSO was used as the negative control. Compound activity for the THR-β/RXR-α and THR-α/RXR-α assays were normalized to T3 activity for each assay run. THR-β selectivity was calculated by dividing the normalized THR-β/RXR-α compound activity by the normalized THR-α/RXR-α compound activity. Data using the RXR Heterodimer assay for certain compounds disclosed herein are listed in Table 3.

TABLE 3

| | EC₅₀ THRß- | EC₅₀ THRα- | |
|---|---|---|---|
| Example | het [μM] | het [μM] | THRß-Selectivity |
| 1 | 0.75 | 4.6 | 5.1 |
| 2 | 0.09 | 1.9 | 16.9 |
| 3 | 0.20 | 2.2 | 9.7 |
| 4 | 0.21 | 2.4 | 9.9 |
| 5 | 0.04 | 0.3 | 5.8 |
| 6 | 0.13 | 2.5 | 16.9 |
| 7 | 0.20 | 4.5 | 21.2 |
| 8 | 0.33 | 3.2 | 7.2 |
| 9 | 0.15 | 3.0 | 16.5 |
| 10 | 0.29 | 3.3 | 7.7 |
| 11 | 0.06 | 1.4 | 17.7 |
| 12 | 0.04 | 0.7 | 18.7 |
| 13 | 0.21 | 3.0 | 11.0 |
| 14 | 0.06 | 0.9 | 12.0 |
| 15 | 0.03 | 0.4 | 10.3 |
| 16 | 0.10 | 1.2 | 9.8 |
| 17 | 0.20 | 2.4 | 10.1 |
| 18 | 0.06 | 0.6 | 8.2 |
| 19 | 0.46 | 3.3 | 5.3 |
| 20 | 0.32 | 0.76 | 2.4 |
| 21 | 0.08 | 0.23 | 2.9 |
| 22 | 0.07 | 0.27 | 3.9 |
| 23 | 0.07 | 0.31 | 4.4 |
| 24 | 0.48 | 3.23 | 6.7 |
| 25 | 0.02 | 0.11 | 5.5 |
| 26 | 1.26 | 2.5 | 2.0 |
| 27 | 0.66 | 0.72 | 1.1 |

### Example B3

### Pharmacokinetics Study

Examples 12 and 14 were administered via a 30-minute intravenous (IV) infusion at approximately 1 mg/kg and orally at approximately 3 mg/kg to male Sprague-Dawley (SD) rats. The concentrations of Examples 12 and 14 in rat plasma was determined with an LC-MS/MS method.

Following IV infusion administration of Example 12 at 1.09 mg/kg in male SD rats (n=3), Example 12 showed a plasma clearance (CL) of 4.51±0.883 mL/min/kg (mean±SD), and half-life of 2.67±0.53 hours. The volume of distribution (Vdₛₛ) was 0.638±0.218 L/kg and the area under the curve AUC_{0-inf} was 3780.0±704.0 ng·h/mL.

Following oral administration of Example 12 at 3.0 mg/kg in male SD rats, the AUC₀₋ₗₐₛₜ and AUC_{0-inf} values were both 10800.0±5630.0 ng·h/mL. Example 12 reached Cₘₐₓ of 2020.0±793.0 ng/mL at 3.33±1.15 hours post-dose. The mean oral bioavailability of Example 12 was estimated to be 95.2% in this species.

Following IV infusion administration of Example 14 at 1.09 mg/kg in male SD rats (n=3), Example 14 showed a plasma clearance (CL) of 30.3±1.07 mL/min/kg (mean±SD), and half-life of 1.51±0.54 hours. The volume of distribution (Vdss) was 1.28±0.387 L/kg and the area under the curve AUC_{0-inf} was 550.0±19.6 ng·h/mL.

Following oral administration of Example 14 at 3.18 mg/kg in male SD rats, the AUC₀₋ₗₐₛₜ and AUC_{0-inf} values were 178.0±25.5 and 190.0±28.7 ng·h/mL, respectively. Example 14 reached Cmax of 31.0 ±5.92 ng/mL at 4.0 ±2.0 hours post-dose. The mean oral bioavailability of Example 14 was estimated to be 11.5% in this species.

### Example B4

### Tissue Distribution

Examples 12 and 14 were administered via a 30-minute intravenous (IV) infusion at approximately 2 mg/kg to jugular vein cannulated male Sprague-Dawley (SD) rats. All animals were euthanized by CO₂ inhalation at 2 hours post-dose. The concentrations of Examples 12 and 14 in rat plasma and liver, heart and kidney tissues were determined with an LC-MS/MS method.

Following IV infusion administration of Example 12 at 2.0 mg/kg in male SD rats (n=3), Example 12 was distributed to all tissues (liver, heart and kidney) at 2 hours post-dose. The highest tissue concentration was observed in liver (3040.0±350.0 ng/g, mean±SD), followed by heart (995.0±1430.0 ng/g) and kidney (558.0±103.0 ng/g). The plasma concentration at 2 hours post-dose was 569.0±145.0 ng/mL. The tissue/plasma ratio of liver/plasma, heart/plasma and kidney/plasma was 5.62±1.09, 1.65±2.33, and 1.01±0.212, respectively.

Following IV infusion administration of Example 14 at 2.16 mg/kg in male SD rats (n=3), Example 14 was distributed to liver and kidney tissues at 2 hours post-dose but was below the limit of detection in heart tissue at that timepoint. The highest tissue concentration was observed in liver (413.0±303.0 ng/g), followed by kidney (94.0±23.5 ng/g). The plasma concentration at 2 hours post-dose was 15.5±3.7 ng/mL. The tissue/plasma ratio of liver/plasma, and kidney/plasma was 29.0±24.9, and 6.25±2.13, respectively.

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

1. A compound of formula (I): 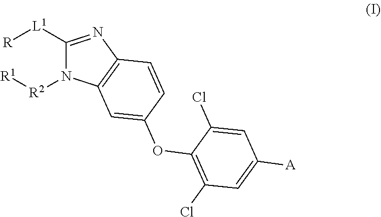
or a tautomer or stereoisomer thereof, or a pharmaceutically acceptable salt of each of the foregoing, wherein:
A is 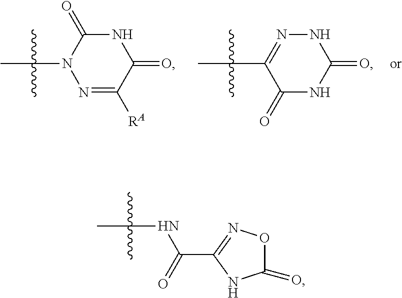
wherein R^{A} is H or —CN;
L¹ is a bond, —NR′—, —O—, —S—, or —S(O)₂—, wherein R′ is H or C₁-C₆ alkyl;
L² is a bond or —S(O)₂—;
R¹ is H, C₁-C₆ alkyl, C₆-C₁₀ aryl, 3-12 membered heterocyclyl, 5-12 membered heteroaryl, or C₃-C₆ cycloalkyl, wherein the C₁-C₆ alkyl, C₆-C₁₀ aryl, 3-12 membered heterocyclyl, 5-12 membered heteroaryl, and C₃-C₆ cycloalkyl are each independently optionally substituted by 1-5 R² groups;
R is H, C₁-C₆ alkyl, C₆-C₁₀ aryl, 3-12 membered heterocyclyl, 5-12 membered heteroaryl, or C₃-C₆ cycloalkyl, wherein the C₁-C₆ alkyl, C₆-C₁₀ aryl, 3-12 membered heterocyclyl, 5-12 membered heteroaryl, and C₃-C₆ cycloalkyl are each independently optionally substituted by 1-5 R² groups; and
each R² is independently halogen, C₁-C₆ alkyl, C₃-C₆ cycloalkyl, C₁-C₆ haloalkyl, C₁-C₆ alkyl-OH, —NH₂, —CN, or hydroxyl, provided that
when L¹ is a bond and R is H, then A is 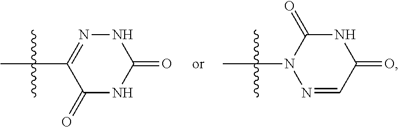 [SMILES: *c1n[nH]c(=O)[nH]c1=O ; *n1ncc(=O)[nH]c1=O]
and
when L¹ is —O—, R is H, and A is 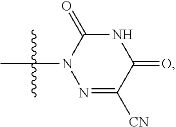 [SMILES: *n1nc(C#N)c(=O)[nH]c1=O]
then R¹ is C₂-C₆ alkyl, C₆-C₁₀ aryl, 3-12 membered heterocyclyl, 5-12 membered heteroaryl, or C₃-C₆ cycloalkyl, wherein the C₂-C₆ alkyl, C₆-C₁₀ aryl, 3-12 membered heterocyclyl, 5-12 membered heteroaryl, and C₃-C₆ cycloalkyl are each independently optionally substituted by 1-5 R² groups.

2. The compound of claim 1, or a tautomer or stereoisomer thereof, or a pharmaceutically acceptable salt of each of the foregoing, wherein
A is

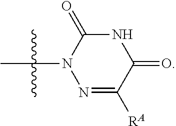

3. The compound of claim 1, or a tautomer or stereoisomer thereof, or a pharmaceutically acceptable salt of each of the foregoing, wherein
A is

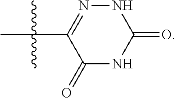 [SMILES: *c1n[nH]c(=O)[nH]c1=O]

4. The compound of claim 1, or a tautomer or stereoisomer thereof, or a pharmaceutically acceptable salt of each of the foregoing, wherein
A is

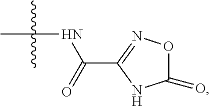 [SMILES: *NC(=O)c1noc(=O)[nH]1]

5. The compound of any one of claims 1-4, or a tautomer or stereoisomer thereof, or a pharmaceutically acceptable salt of each of the foregoing, wherein L¹ is a bond.

6. The compound of any one of claims 1-4, or a tautomer or stereoisomer thereof, or a pharmaceutically acceptable salt of each of the foregoing, wherein L¹ is —NR′—.

7. The compound of any one of claims 1-4, or a tautomer or stereoisomer thereof, or a pharmaceutically acceptable salt of each of the foregoing, wherein L¹ is —O—.

8. The compound of any one of claims 1-4, or a tautomer or stereoisomer thereof, or a pharmaceutically acceptable salt of each of the foregoing, wherein L¹ is —S—.

9. The compound of any one of claims 1-4, or a tautomer or stereoisomer thereof, or a pharmaceutically acceptable salt of each of the foregoing, wherein L¹ is —S(O)₂—.

10. The compound of any one of claims 1-9, or a tautomer or stereoisomer thereof, or a pharmaceutically acceptable salt of each of the foregoing, wherein L² is a bond.

11. The compound of any one of claims 1-9, or a tautomer or stereoisomer thereof, or a pharmaceutically acceptable salt of each of the foregoing, wherein L² is —S(O)₂—.

12. The compound of any one of claims 1-11, or a tautomer or stereoisomer thereof, or a pharmaceutically acceptable salt of each of the foregoing, wherein R is H.

13. The compound of any one of claims 1-11, or a tautomer or stereoisomer thereof, or a pharmaceutically acceptable salt of each of the foregoing, wherein R is C₁-C₆ alkyl optionally substituted by 1-3 R² groups.

14. The compound of any one of claims 1-11, or a tautomer or stereoisomer thereof, or a pharmaceutically acceptable salt of each of the foregoing, wherein R is methyl or ethyl, each of which is optionally substituted by 1-3 R² groups.

15. The compound of any one of claims 1-14, or a tautomer or stereoisomer thereof, or a pharmaceutically acceptable salt of each of the foregoing, wherein R¹ is H.

16. The compound of any one of claims 1-14, or a tautomer or stereoisomer thereof, or a pharmaceutically acceptable salt of each of the foregoing, wherein R¹ is C₁-C₆ alkyl which is optionally substituted by 1-5 R² groups.

17. The compound of any one of claims 1-14, or a tautomer or stereoisomer thereof, or a pharmaceutically acceptable salt of each of the foregoing, wherein R¹ is methyl, ethyl, isopropyl, or t-butyl.

18. The compound of any one of claims 1-14, or a tautomer or stereoisomer thereof, or a pharmaceutically acceptable salt of each of the foregoing, wherein R¹ is C₃-C₆ cycloalkyl optionally substituted by 1-5 R² groups.

19. The compound of any one of claims 1-14, or a tautomer or stereoisomer thereof, or a pharmaceutically acceptable salt of each of the foregoing, wherein R¹ is cyclopropyl or cyclobutyl, each of which is optionally substituted by 1 R² group.

20. The compound of any one of claims 1-14, or a tautomer or stereoisomer thereof, or a pharmaceutically acceptable salt of each of the foregoing, wherein R¹ is

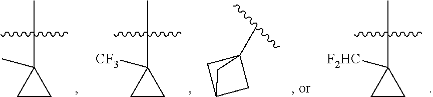

21. The compound of claim 1, or a tautomer or stereoisomer thereof, or a pharmaceutically acceptable salt of each of the foregoing, wherein the compound is selected from the compounds in Table 1.

22. A pharmaceutical composition, comprising the compound of any one of claims 1-21, or a tautomer or stereoisomer thereof, or a pharmaceutically acceptable salt of each of the foregoing, and a pharmaceutically acceptable excipient.

23. A method of treating a disorder ameliorated by activation of thyroid hormone receptor beta, comprising administering a therapeutically effective amount of the compound of any one of claims 1-21, or a tautomer or stereoisomer thereof, or a pharmaceutically acceptable salt of each of the foregoing, or the pharmaceutical composition of claim 22, to a patient in need thereof.

24. The method of claim 23, wherein the disorder is non-alcoholic steatohepatitis (NASH), non-alcoholic fatty liver disease (NAFLD), metabolic syndrome, dyslipidemia, hypertriglyceridemia, or hypercholesterolemia.

25. The method of claim 24, wherein the disorder is NASH.
